(19) United States
(12) Patent Application Publication
(10) Pub. No.: US 2016/0074041 A1
(43) Pub. Date: Mar. 17, 2016

(54) URETHRAL ANASTOMOSIS DEVICE AND METHOD

(71) Applicant: ENDO PHARMACEUTICALS INC., Malvern, PA (US)

(72) Inventor: Anthony J. Wirtel, III, Malvern, PA (US)

(21) Appl. No.: 14/775,281

(22) PCT Filed: Mar. 10, 2014
(86) PCT No.: PCT/US2014/022435

§ 371 Date: Sep. 11, 2015

Related U.S. Application Data
(60) Provisional application No. 61/783,131, filed on Mar. 14, 2013.

(51) Int. Cl. A61B 17/11; A61B 17/064

(52) U.S. Cl. CPC A61B 17/11; A61B 17/0643; A61B 2017/1132; A61B 17/115

(57) ABSTRACT

Provided herein is an anastomosis assembly for connecting a first tissue portion to a second tissue portion. The anastomosis assembly includes a first anastomosis portion having first tissue engaging structures for deployment, by actuation of a deployment mechanism of a deployment device, to attach to the first tissue portion, and a second anastomosis portion having second tissue engaging structures for deployment, by actuation of a deployment mechanism of a deployment device, to attach to the second tissue portion. During delivery of the anastomosis assembly, the first and second tissue engaging structures are contained within an inner diameter of the first and second anastomosis portions.

### TECHNICAL FIELD

This disclosure relates generally to the field of medical devices and, in particular, to devices and methods for reconnecting two hollow body parts, such as a urethra to a bladder.

### BACKGROUND

The prostate gland is a semen-producing organ located in the abdomen of males. Cancer of the prostate gland is an extremely common ailment among older American men. In fact, prostate cancer is the second-leading cause of cancer-related deaths and the most common cancer diagnosed in men. In 2010, an estimated 90,000 American men underwent radical prostatectomy, a surgery in which their prostate gland was removed. If past experience holds, nearly one-third of these men suffered complications, which at the least were painful and at most required further invasive surgery.

The most common complication, known as bladder-neck contracture, is caused by leakage of urine into the abdomen. During a radical prostatectomy, after the prostate is removed, it is necessary to re-attach the bladder (where the body stores urine) to the urethra (the passage carrying urine from the bladder to the penis). Unfortunately, the conventional hand-sewn five- to six-suture re-attachment (an anastomosis) often does not result in a leak-proof seal. Consequently, urine can leak from the bladder into the abdomen until the anastomosis is sealed, which can take up to five days. Such leakage causes scarring, which in turn leads to bladder-neck contractures. A patient suffering from such a contracture typically is unable to urinate and requires painful and expensive intervention.

In addition, with the robotic approach, the urethrovesicle anastomosis can be one of the most challenging components of the surgery. In the most-experienced hands, this can add thirty minutes to the operation, and in the hands of a novice, it can add one hour to the operation.

Accordingly, it can be seen that a need exists for improved ways to attach hollow body vessels, such as the urethra to the bladder. It is to this and other solutions that the embodiments of the present invention are primarily directed.

### BRIEF DESCRIPTION OF THE DRAWINGS

FIG. 1 is a perspective view of a first exemplary embodiment of a first ring assembly structure of an anastomosis device.

FIG. 2 is a further perspective view of the first ring assembly of FIG. 1.

FIG. 3 is a further perspective view of the first ring assembly of FIG. 1.

FIG. 4 is a cross-sectional view of the first ring assembly of FIG. 1, depicted in the retracted position.

FIG. 5 is a cross-sectional view of the first ring assembly of FIG. 1, depicted in the deployed position.

FIG. 6 is a perspective view of a first exemplary embodiment of a second ring assembly structure of an anastomosis device.

FIG. 6A is a perspective view of an alternative embodiment of a portion of the second ring assembly depicted in FIG. 6.

FIG. 6B is a perspective view showing alternative embodiments of a first ring assembly and a second ring assembly.

FIG. 6C is a partial perspective view of an exemplary embodiment showing a first ring assembly coupled to a second ring assembly.

FIG. 7 is a perspective view of a first exemplary embodiment of an anastomosis system.

FIG. 8 is an exploded view of the anastomosis system of FIG. 7.

FIG. 9A is a perspective view of a first exemplary embodiment of an actuation shaft used within an anastomosis device.

FIG. 9B is a further perspective view of the actuation shaft of FIG. 9A.

FIG. 10A is a further perspective view of the actuation shaft of FIGS. 9A and 9B, depicted with an adapter and rotary actuation knob.

FIG. 10B is a further perspective view of the actuation shaft of FIG. 10A.

FIG. 10C is a further perspective view of the actuation shaft of FIG. 10A.

FIG. 11 is a perspective view of the actuation shaft of FIGS. 9A and 9B, depicted with an adapter and rotary selection knob.

FIG. 12A is a perspective view of a first exemplary embodiment of a partially assembled exemplary handle assembly for an anastomosis device.

FIG. 12B is a further perspective view of the handle assembly of FIG. 12A.

FIG. 13A is a perspective view of a first exemplary embodiment of an implant support.

FIG. 13B is a further perspective view of the implant support of FIG. 13A.

FIG. 13C is a cross-sectional view of the implant support shown in FIGS. 13A and 13B.

FIG. 14A is a perspective view of the actuation shaft shown in FIGS. 10A-10C, depicted during a first stage of a deployment operation.

FIG. 14B is a perspective view of the actuation shaft shown in FIG. 14A, depicted during a second stage of a deployment operation.

FIG. 14C is a perspective view of the actuation shaft shown in FIG. 14A, depicted during a third stage of a deployment operation.

FIG. 14D is a perspective view of the actuation shaft shown in FIG. 14A, depicted during a fourth stage of a deployment operation.

FIG. 14E is a perspective view of the actuation shaft shown in FIG. 14A, depicted during a fifth stage of a deployment operation.

FIG. 15A is a cross-sectional view of the handle assembly depicted in FIGS. 12A and 12B.

FIG. 15B is a further cross-sectional view of the handle assembly depicted in FIGS. 12A and 12B.

FIG. 16 is a further perspective view of the anastomosis system depicted in FIG. 7.

FIG. 17A is a cross-sectional view of a distal end of the anastomosis system depicted in FIG. 16.

FIG. 18A is a further cross-sectional view of the distal end of the anastomosis system depicted in FIG. 17A.

FIG. 18B is a further cross-sectional view of the proximal end of the anastomosis system depicted in FIG. 17B.

FIG. 19 is a perspective view of a second exemplary embodiment of an anastomosis system.

FIG. 20 is a perspective view of a third exemplary embodiment of an anastomosis system.

FIG. 21 is a perspective view of a shaft flexing portion of the anastomosis system of FIG. 20.

FIG. 22 is a perspective view of a fourth exemplary embodiment of an anastomosis system.

FIG. 23A is a perspective view of a second exemplary embodiment handle assembly for use with an anastomosis system.

FIG. 23B is a side view of the handle assembly shown in FIG. 23A.

FIG. 24A is a perspective view of a third exemplary embodiment handle assembly for use with an anastomosis system.

FIG. 24B is a side view of the handle assembly shown in FIG. 24A.

FIG. 25 is a further perspective view of the anastomosis system of FIG. 7, depicted during insertion into a patient.

FIG. 26A is a further perspective view of the anastomosis system depicted in FIG. 25, during a first stage of the insertion and deployment process.

FIG. 26B is a cross-sectional view of the anastomosis system shown in FIG. 26A.

FIG. 26C is a cross-sectional view of a handle portion of the anastomosis system of FIG. 26A.

FIG. 26D is a cross-sectional view of a distal portion of the anastomosis system of FIG. 26A.

FIG. 27A is a further perspective view of the anastomosis system depicted in FIG. 25, during a second stage of the insertion and deployment process.

FIG. 27B is a cross-sectional view of the anastomosis system shown in FIG. 27A.

FIG. 27C is a cross-sectional view of a handle portion of the anastomosis system of FIG. 27A.

FIG. 27D is a cross-sectional view of a distal portion of the anastomosis system of FIG. 27A.

FIG. 28A is a further perspective view of the anastomosis system depicted in FIG. 25, during a third stage of the insertion and deployment process.

FIG. 28B is a cross-sectional view of the anastomosis system shown in FIG. 28A.

FIG. 28C is a cross-sectional view of a handle portion of the anastomosis system of FIG. 28A.

FIG. 28D is a cross-sectional view of a distal portion of the anastomosis system of FIG. 28A.

FIG. 29A is a further perspective view of the anastomosis system depicted in FIG. 25, during a fourth stage of the insertion and deployment process.

FIG. 29B is a cross-sectional view of the anastomosis system shown in FIG. 28A.

FIG. 29C is a cross-sectional view of a handle portion of the anastomosis system of FIG. 29A.

FIG. 29D is a cross-sectional view of a distal portion of the anastomosis system of FIG. 29A.

FIG. 30A is a further perspective view of the anastomosis system depicted in FIG. 25, during a fifth stage of the insertion and deployment process.

FIG. 30B is a cross-sectional view of the anastomosis system shown in FIG. 30A.

FIG. 30C is a cross-sectional view of a handle portion of the anastomosis system of FIG. 30A.

FIG. 30D is a cross-sectional view of a distal portion of the anastomosis system of FIG. 30A.

FIG. 31A is a further perspective view of the anastomosis system depicted in FIG. 25, during a sixth stage of the insertion and deployment process.

FIG. 31B is a cross-sectional view of the anastomosis system shown in FIG. 31A.

FIG. 31C is a cross-sectional view of a handle portion of the anastomosis system of FIG. 31A.

FIG. 31D is a cross-sectional view of a distal portion of the anastomosis system of FIG. 31A.

FIG. 32A is a further perspective view of the anastomosis system depicted in FIG. 25, during a seventh stage of the insertion and deployment process.

FIG. 32B is a cross-sectional view of the anastomosis system shown in FIG. 32A.

FIG. 32C is a cross-sectional view of a handle portion of the anastomosis system of FIG. 32A.

FIG. 32D is a cross-sectional view of a distal portion of the anastomosis system of FIG. 32A.

FIG. 33A is a side view of a portion of a further alternative exemplary embodiment of a central ring in a retracted or undeployed position.

FIG. 33B is a side view of a portion of the central ring depicted in FIG. 33A in an extended or deployed position.

FIG. 34 is a perspective view of a further alternative embodiment of a first ring assembly in the undeployed position.

FIG. 35A is a side view of an alternative embodiment of a first ring securement element.

FIG. 35B is a side view of an alternative embodiment of a first ring securement element.

FIG. 35C is a side view of a further alternative embodiment of a first ring securement element.

FIG. 35D is a side view of an alternative embodiment of a first ring securement element.

FIG. 36A is a cross-sectional view of an alternative embodiment of a first ring assembly in an undeployed position.

FIG. 36B is a cross-sectional view of the alternative embodiment of a first ring assembly depicted in FIG. 36A in a partially deployed position.

FIG. 36C is a cross-sectional view of the alternative embodiment of a first ring assembly depicted in FIG. 36A in a fully deployed position.

FIG. 37A is a cross-sectional view of a further alternative embodiment of a distal portion of an anastomosis system.

FIG. 37B is a cross-sectional view of the distal portion of an anastomosis system depicted in FIG. 37A, after release of the ring assembly from the insertion instrument.

FIG. 37C is a cross-sectional view of the distal portion of an anastomosis system depicted in FIG. 37A, after withdrawal of the insertion instrument.

FIG. 38A is a cross-sectional view of a further alternative embodiment of a distal portion of an anastomosis system, with a shaft flexing portion.

FIG. 38B is a cross-sectional view of the distal portion of an anastomosis system depicted in FIG. 38A, with the shaft flexing portion during flexing.

FIG. 38C is a cross-sectional view of the distal portion of an anastomosis system depicted in FIG. 38A, with the shaft flexing portion during further flexing.

FIG. 39 is a perspective view of the distal portion of a further alternative embodiment of an anastomosis system with the second ring assembly in the undeployed position.

FIG. 40 is a perspective view of an anastomosis system depicted in FIG. 39, with the second ring assembly in the partially deployed position.

FIG. 41 is a perspective view of an anastomosis system depicted in FIG. 39, with the second ring assembly in the fully deployed position.

FIG. 42 is a perspective view of a ring assembly of the alternative anastomosis system depicted in FIG. 39, in the fully deployed position.

FIG. 43 is a side view of a further alternative embodiment of an anastomosis system with the second central ring mounted proximally with respect to the second collar.

FIG. 44 is a perspective view of a further alternative embodiment of an anastomosis system with the second central ring mounted proximally with respect to the second collar.

FIG. 45A is a cross-sectional view of a further alternative embodiment of an anastomosis device shown in various positions with respect to the tissue of a patient.

FIG. 45B is another cross-sectional view of the embodiment of an anastomosis device shown in FIG. 45A.

FIG. 46A shows a perspective view of a further embodiment of an anastomosis device shown in an undeployed position.

FIG. 46B shows the anastomosis device of FIG. 46A in the deployed position.

FIG. 47 is a cross-sectional view of a further alternative embodiment of an anastomosis device, shown in various stages of deployment.

FIG. 48A is a cross sectional view of a further alternative embodiment of an anastomosis device, shown in various stages (1-4) of deployment.

FIG. 48B is a perspective view of the anastomosis device of FIG. 48A, shown in connection with a portion of a patient's vessel, such as a bladder.

FIG. 49 is a side view of a further alternative embodiment of an anastomosis device shown in various stages (1-3) of deployment.

FIG. 50 is a perspective view of a further alternative embodiment of an anastomosis device shown in various stages (1-3) of deployment.

FIG. 51 is a side view of a further alternative embodiment of an anastomosis device, shown in various stages (1-3) of deployment.

FIG. 52A depicts corresponding perspective and cross-sectional views of a further alternative embodiment of an anastomosis device, shown in various stages (1-2) of deployment.

FIG. 52B depicts a partially exploded view of the anastomosis device of FIG. 52A.

FIG. 53A is a perspective view of a further alternative embodiment of an anastomosis device, shown in an un-deployed state.

FIG. 53B is a perspective view of the anastomosis device of 53A, shown in a deployed state.

FIG. 54A provides top plan views of a portion of a further alternative embodiment of an anastomosis device, shown in various stages (1-2) of deployment.

FIG. 54B is a perspective view of the anastomosis device of 54A, shown in various stages (1-3) of deployment.

FIG. 55 is a perspective view of a further alternative embodiment of an insertion device, shown in a closed position.

FIG. 56 is a perspective view of a further alternative embodiment of an insertion device, shown in a various stages (1-2) of articulation.

FIG. 57A is a perspective view of a further alternative embodiment of an anastomosis device, shown in a deployed position.

FIG. 57B is another perspective view of the anastomosis device of FIG. 57A, shown in the deployed position.

FIG. 58 is a side view of a further alternative embodiment of a tissue engagement structure, shown in various stages (1-2) of deployment.

FIG. 59 is a side view of a further alternative embodiment of a tissue engagement structure, shown in various stages (1-2) of deployment.

FIG. 60A is a top plan view of a further alternative embodiment of an anastomosis device, shown in various stages (1-2) of deployment.

FIG. 60B is a side view the anastomosis device of FIG. 60A, shown in various stages (1-2) of deployment.

FIG. 60C is a perspective view of the anastomosis device of FIG. 60A, shown in a deployed position.

FIG. 61 is a top plan view of a further alternative embodiment of an anastomosis device, shown in an undeployed state.

FIG. 62A is a side plan view of a further alternative embodiment of an anastomosis device, shown in a deployed, but un-retracted state.

FIG. 62B is a side view the anastomosis device of FIG. 62A, shown in place in a bladder and urethra in a deployed, but un-retracted state.

FIG. 62C is a side view the anastomosis device of FIG. 62A, shown in place in a bladder and urethra in a deployed and retracted state.

FIG. 63A is a side view of a further alternative embodiment of an anastomosis device, shown in various stages (1-2) of deployment.

FIG. 63B is a side view the anastomosis device of FIG. 63A, shown in place in a bladder and urethra in a deployed, but un-retracted state.

FIG. 63C is a side view the anastomosis device of FIG. 63A, shown in place in a bladder and urethra in a deployed and retracted state.

FIG. 64A is a side view of a further alternative embodiment of an anastomosis device, shown in an un-deployed state.

FIG. 64B is a side view of a portion of the anastomosis device of FIG. 64A, shown in both the un-deployed and deployed state.

FIG. 64C is a side view the anastomosis device of FIG. 64A, shown in place in a bladder and urethra in an deployed, but un-retracted state. The anastomosis device 3800 is shown here engaged to bladder and urethra tissue, but with the first and second implant rings 3804, 3806 separated.

FIG. 64D is a side view the anastomosis device of FIG. 64A, shown in place in a bladder and urethra in a deployed and retracted state.

FIG. 65A is a perspective view of a further alternative embodiment of an anastomosis device, shown in an un-deployed state.

FIG. 65B is a cross-sectional view of a portion of the anastomosis device of FIG. 65A, shown in a deployed state.

FIG. 65C is a side view the anastomosis device of FIG. 65A, shown in place in a bladder and urethra in an deployed, but un-retracted state.

FIG. 65D is a side view the anastomosis device of FIG. 65A, shown in place in a bladder and urethra in a deployed and retracted state.

FIG. 66A is a side plan view of a further alternative embodiment of an anastomosis device, shown in a deployed, but un-retracted state.

FIG. 66B is a side view the anastomosis device of FIG. 66A, shown in place in a bladder and urethra in an deployed, but un-retracted state.

FIG. 66C is a side view the anastomosis device of FIG. 66A, shown in place in a bladder and urethra in a deployed and retracted state.

FIG. 67A is a side view of a further alternative embodiment of an anastomosis device, shown in a deployed state.

FIG. 67B is a side view of a portion of the anastomosis device of FIG. 67A, shown in place in a bladder and urethra in a partially deployed and un-retracted state.

FIG. 67C is a side view of the anastomosis device of FIG. 67A, shown in place in a bladder and urethra in a deployed and retracted state.

FIG. 68A is a side view of a further alternative embodiment of an anastomosis device, shown in an undeployed state.

FIG. 68B is a cross-sectional view of a portion of the anastomosis device of FIG. 68A, shown in place in a bladder and urethra in a deployed but un-retracted state.

FIG. 68C is a side view of the anastomosis device of FIG. 68A, shown in place in a bladder and urethra in a deployed and retracted state.

FIG. 69A is a cross-sectional view of a further alternative embodiment of an anastomosis device, shown in place in a bladder and urethra in a partially deployed state.

FIG. 69B is an exploded cross-sectional view of a portion of the anastomosis device of FIG. 69A.

FIG. 69C is a cross-sectional view the anastomosis device of FIG. 69A, shown in place in a bladder and urethra in a deployed and retracted state.

FIG. 70A is an exploded view of a further alternative embodiment of an anastomosis device.

FIG. 70B is a side view of the anastomosis device of FIG. 70A shown in place in a bladder and urethra in a deployed but un-retracted state.

FIG. 70C is a side view the anastomosis device of FIG. 70A, shown in place in a bladder and urethra in a deployed and retracted state.

FIG. 71A is a partially exploded perspective view of a further alternative embodiment of an anastomosis device.

FIG. 71B is a side view of the anastomosis device of FIG. 71A, shown in place in a bladder and urethra in a partially deployed state.

FIG. 71C is a side view of the anastomosis device of FIG. 71A, shown in place in a bladder and urethra in a deployed and retracted state.

FIG. 72A is a side view of a further alternative embodiment of an anastomosis device, shown in place in a bladder and urethra in a partially deployed state.

FIG. 72B is a side view of the anastomosis device of FIG. 72A, shown in place in a bladder and urethra in a deployed and retracted state, with an external adhesive applicator.

FIG. 73A is a cross-sectional view of a portion of a further alternative embodiment of an anastomosis device, shown in various stages (1-3) of deployment.

FIG. 73B is a cross-sectional view of a portion of the anastomosis device of FIG. 73A, shown in place in a bladder and urethra in an un-deployed state

FIG. 73C is a side view of the anastomosis device of FIG. 73A, shown in place in a bladder and urethra in a deployed and retracted state.

FIG. 74 is a perspective view of a further alternative embodiment of an anastomosis device, shown in an un-deployed state.

FIG. 75 is a perspective view of a further alternative embodiment of an anastomosis device, shown in a deployed state.

FIG. 76 is a perspective view of a further alternative embodiment of an anastomosis device, shown in an un-deployed state.

FIG. 77 is a perspective view of a further alternative embodiment of an anastomosis device, shown in place in a bladder and urethra in an deployed and retracted state.

FIG. 78 is a perspective view of a further alternative embodiment of an anastomosis device, shown in a partially deployed state.

FIG. 79 is a perspective view of a further alternative embodiment of an anastomosis device, shown in an un-deployed state.

FIG. 80 is a partial perspective view of a further alternative embodiment of an anastomosis device, shown in a deployed state.

FIG. 81 is a partial perspective view of a further alternative embodiment of an anastomosis device, shown in a deployed state.

FIG. 82 is a perspective view of a further alternative embodiment of an anastomosis device, shown in an un-deployed state.

FIG. 83 is a partial perspective view of a further alternative embodiment of an anastomosis device, shown in a deployed but un-retracted state.

FIG. 84 is a perspective view of a further alternative embodiment of an anastomosis device, shown in a deployed but un-retracted state.

### DETAILED DESCRIPTION

The present disclosure generally relates to anastomosis systems and methods. In the depicted embodiments, the systems and methods relate to urethral anastomosis systems and methods. Persons of ordinary skill in the art will appreciate that the teachings herein can be readily adapted to other types of anastomosis systems and methods. Accordingly, as used herein, the terms such as urethra and bladder are not intended to be limiting of the embodiments of the present invention. Instead, it will be understood that the embodiments of the present invention relate generally to the field of medical devices and, in particular to devices and methods for connecting two hollow body parts or vessels, such as the urethra and the bladder, or portions of any other body vessel. As used herein, the terms “proximal” and “distal” refer respectively to the directions closer to and further from the operator of the anastomosis device. For purposes of clarity, the distal portion of the device is inserted furthest into an anastomosis patient and the proximal portion of the device remains closest to the inserting physician. Likewise, the term “lower” is generally used to refer to a proximal portion of the device, i.e. one that is proximally located with respect to a corresponding portion of the device. The term “upper” is generally used to refer to a distal portion of the device, i.e. one that is distally located with respect to a corresponding portion of the device. For frame of reference in the figures, arrows marked “P” refer generally to the proximal direction and arrows marked “D” refer generally to the distal direction relative to the orientation of the items depicted in the figures.

The anastomosis systems of the present disclosure generally include a coupling assembly for connecting and sealing the two body parts and a surgical implement for emplacing the coupling assembly. In typical embodiments, the coupling assembly includes two ring assemblies, with each ring assembly having securement elements that attach to the respective body part and interconnecting elements that attach to the other ring. For example, in some of the depicted embodiments for urethral anastomosis, the coupling assembly includes two ring assemblies each made of a degradable/absorbable material and interconnected to form a leak-proof seal between the bladder and the urethra. When used for urinary anastomosis, the coupling assembly, which may also be referred to as a ring assembly 3 herein, eliminates urine leakage, removing the cause of the most common post-operative complication, bladder-neck contracture. Also, the anastomosis is performed entirely within the urethra and thus there is no risk of damaging the neurovascular bundles that lie directly outside the urethra.

In addition, the surgical instrument of the anastomosis system can be used laparoscopically/robotically as well. Currently, a laparoscopic/robotic prostatectomy requires a hand-sewn urethral anastomosis that can take up to three hours and does not result in an immediate water-tight seal. There has been an enormous increase in robotic-assisted radical prostatectomies during the last five years. This surgical instrument can be used with the present coupling assembly to form a seal between the bladder and the urethra in only approximately fifteen minutes (rather than three hours) and the resulting seal is leak-proof. This system and method also presents the potential to perform the procedure without a urethral catheter, which is normally left in place within a patient for seven to ten days. Finally, the system and method will preferably only compromise about 4-8 mm of urethra, thereby maximizing “functional urethral length,” which is known to be one of the most important determinants of post-operative continence.

In the figures, in which like numerals indicate like elements throughout, there are shown exemplary embodiments of an anastomosis system. The first embodiment of the anastomosis system is generally referred to by the numeral 1.

Ring Assembly

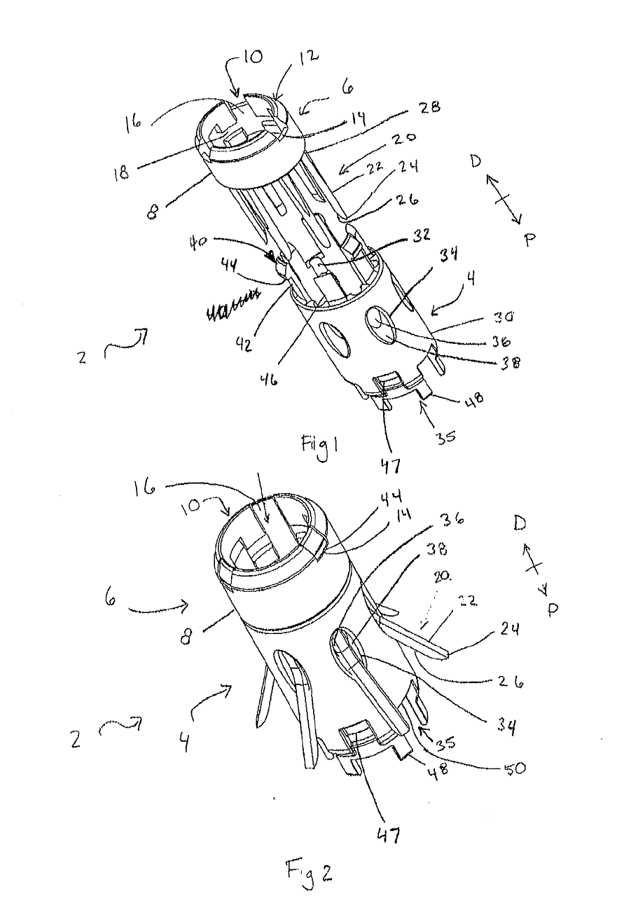

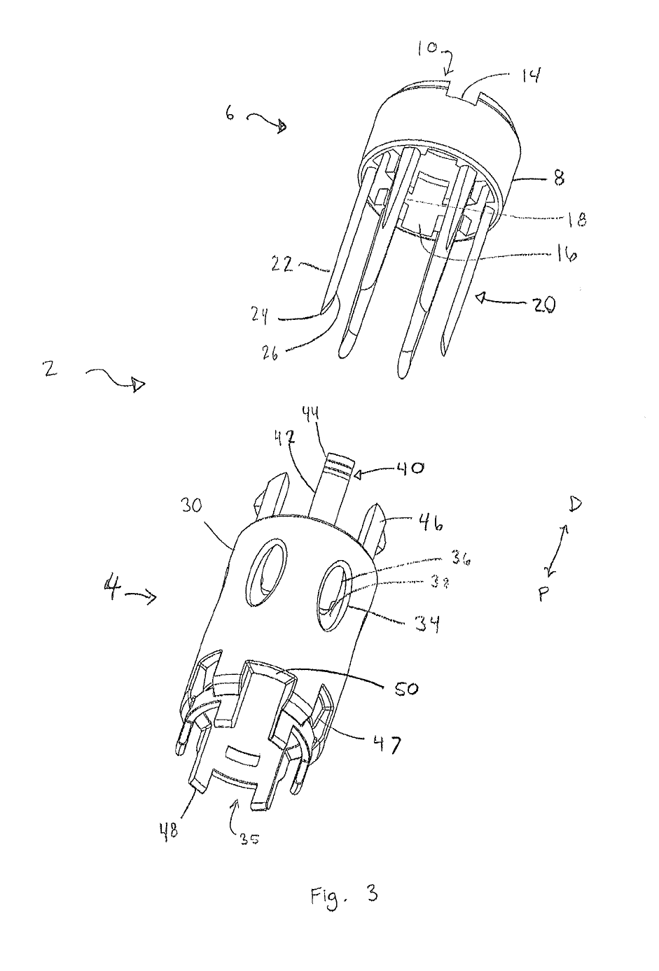

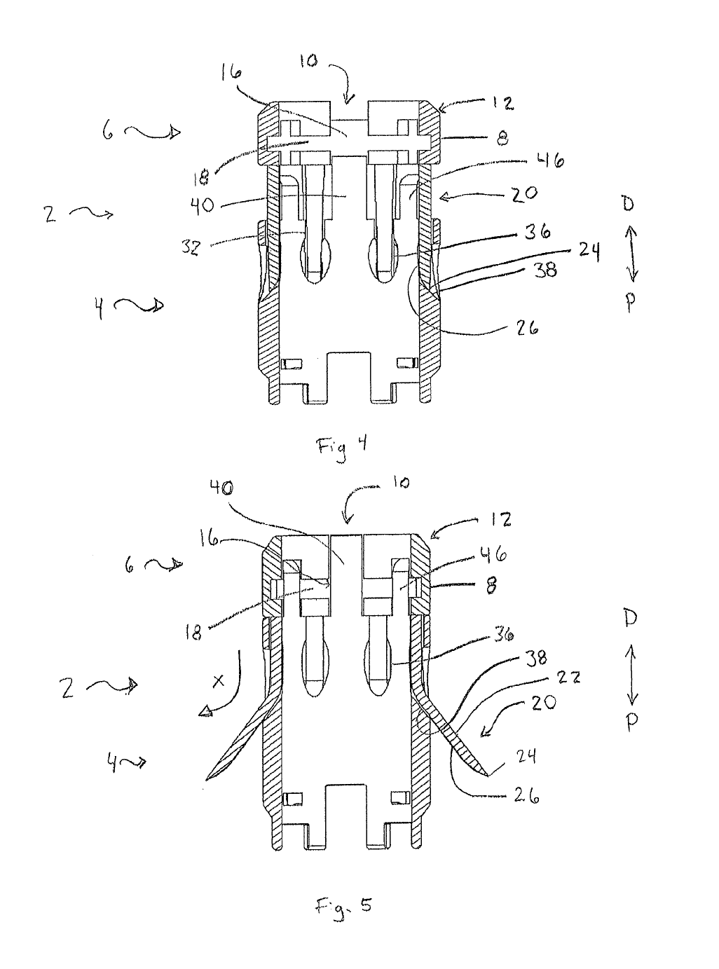

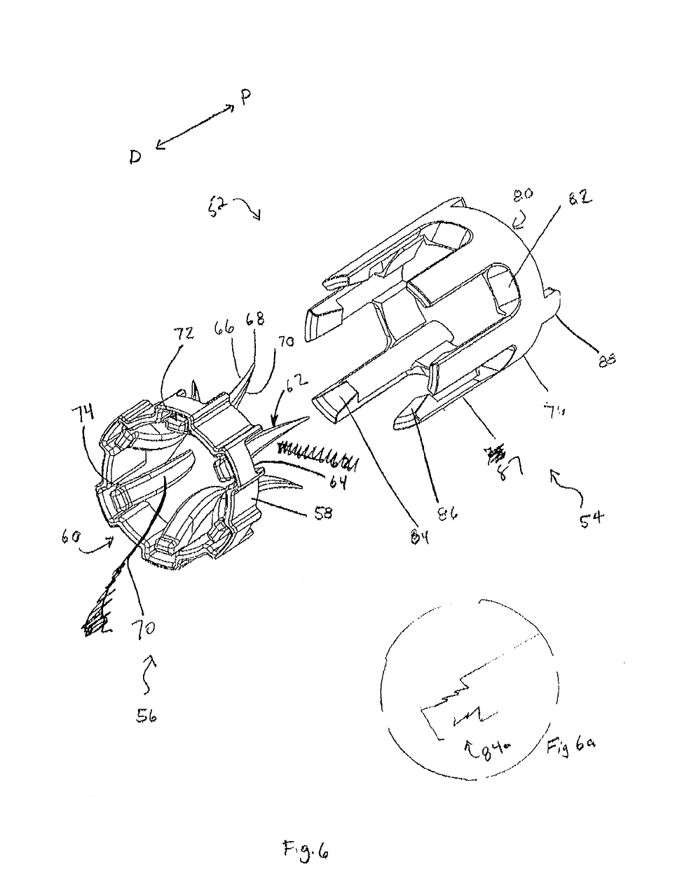

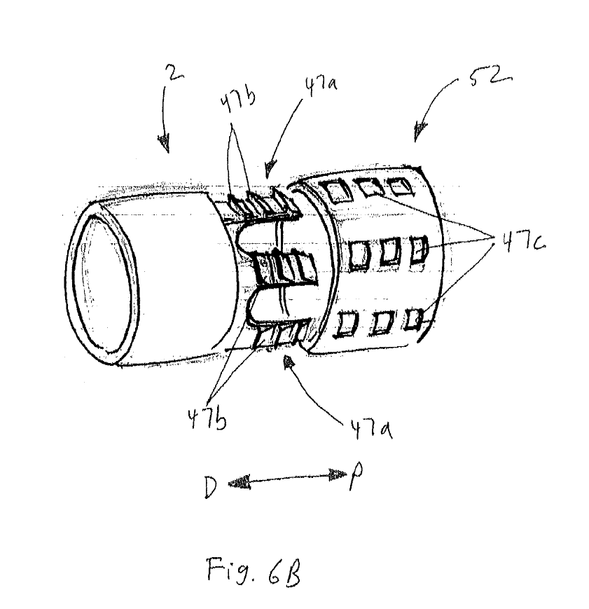

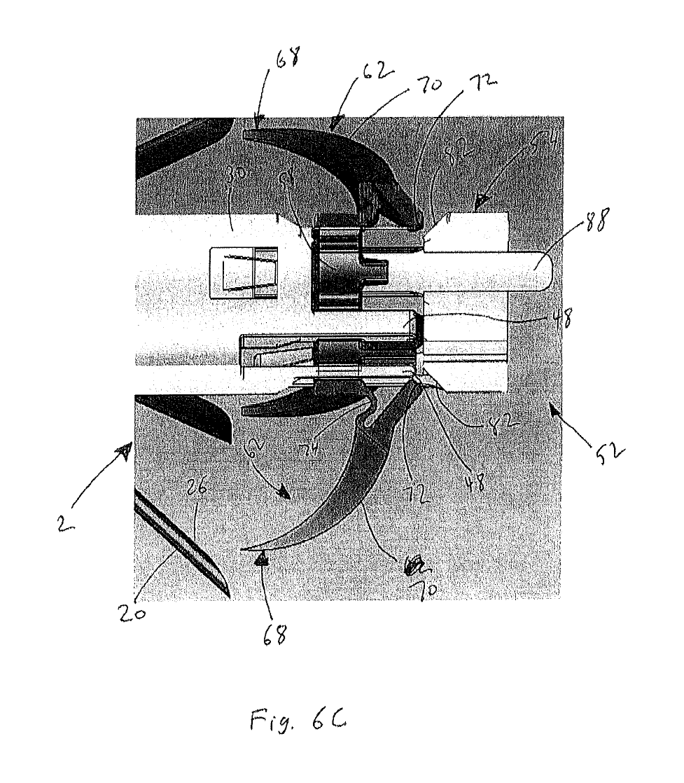

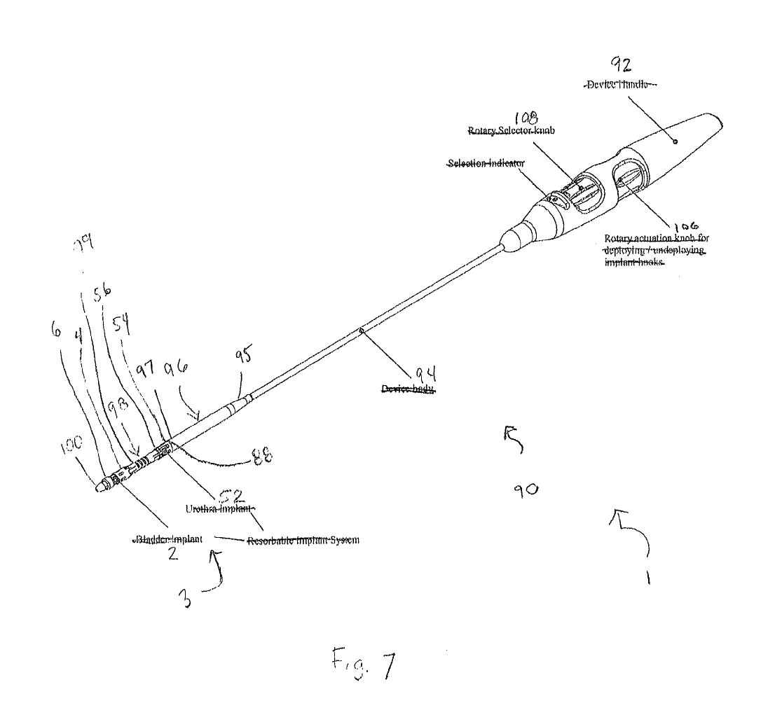

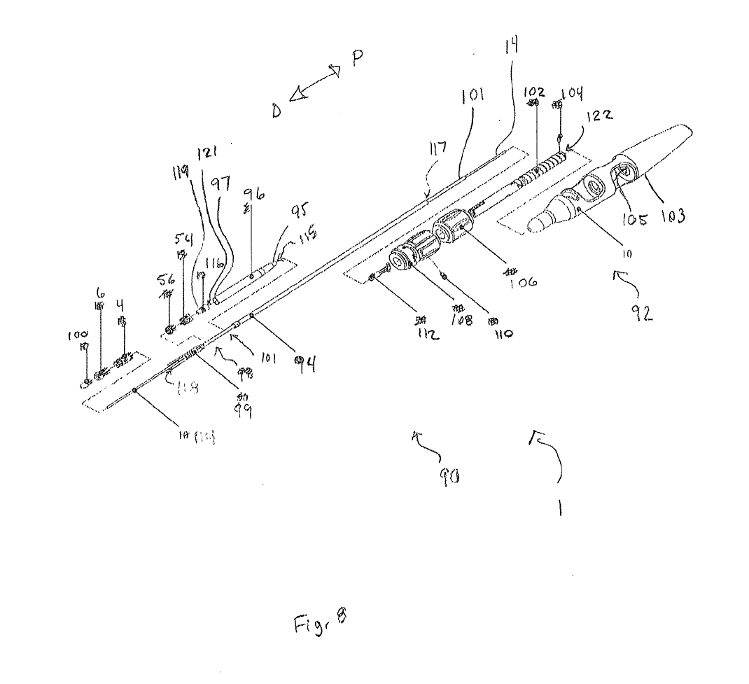

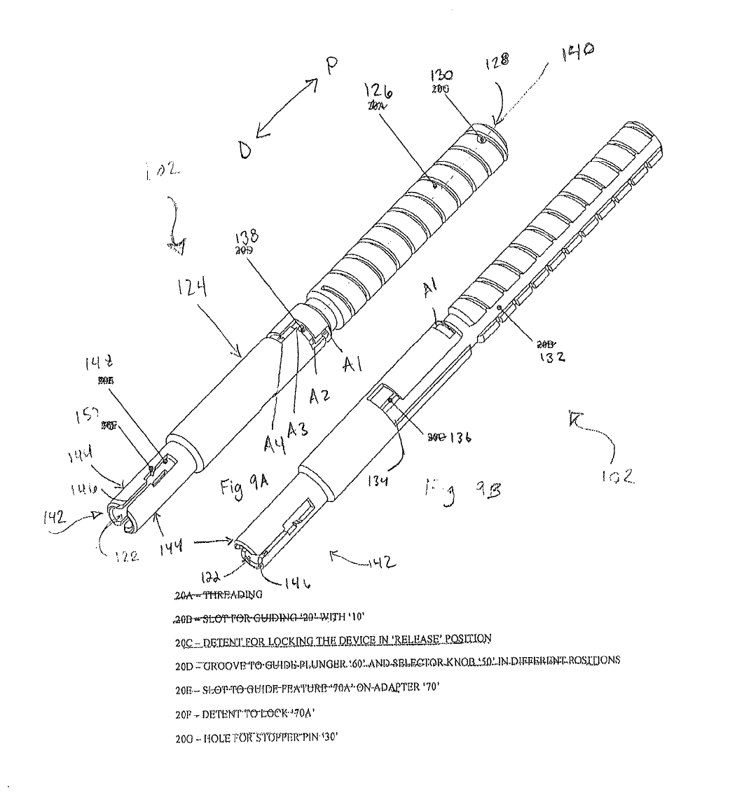

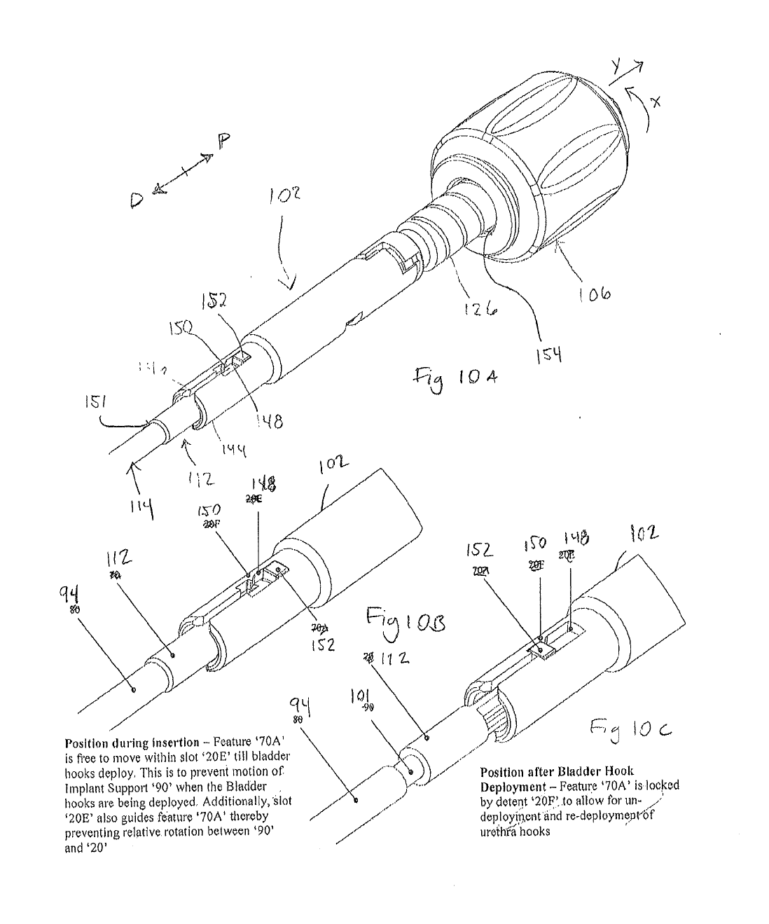

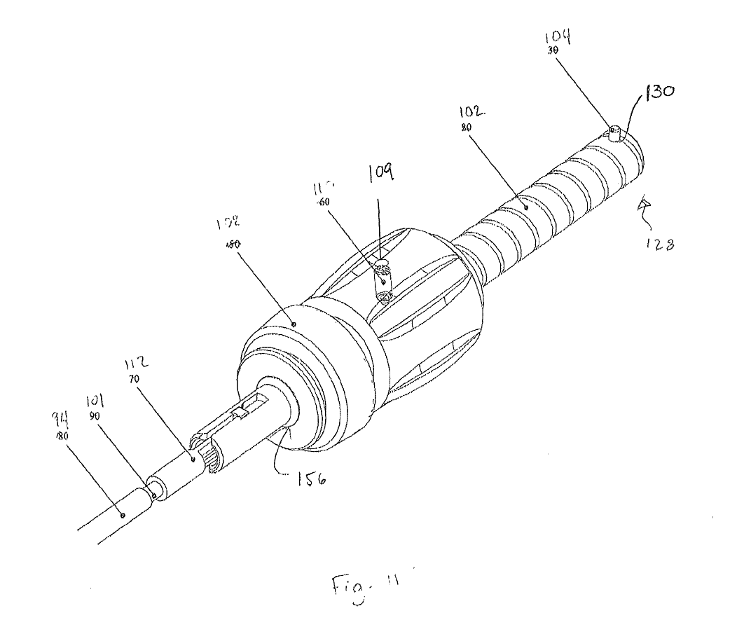

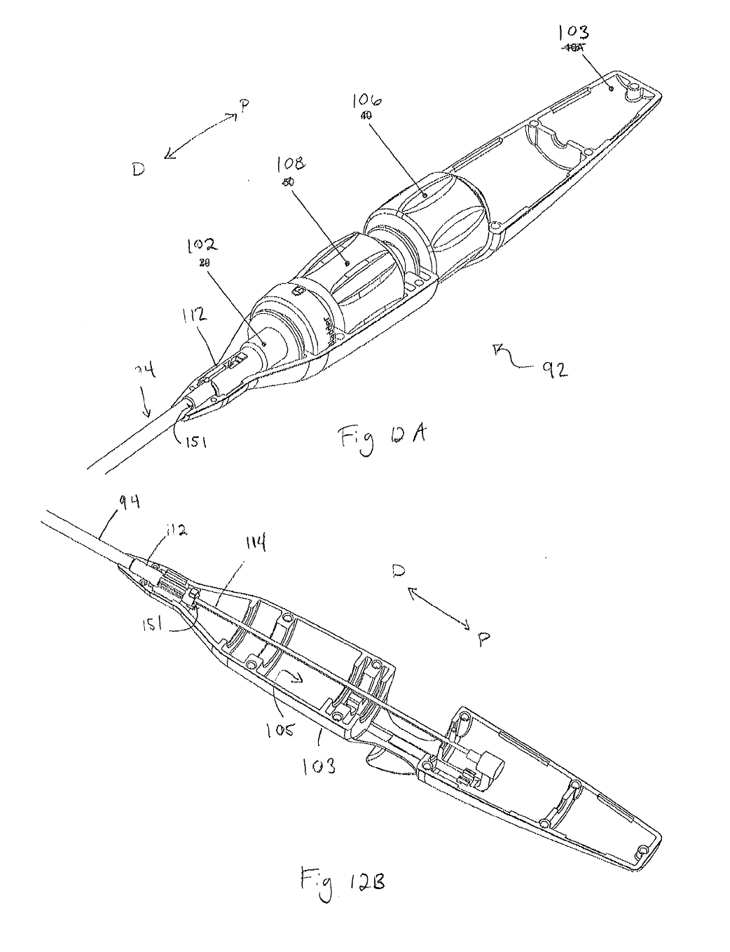

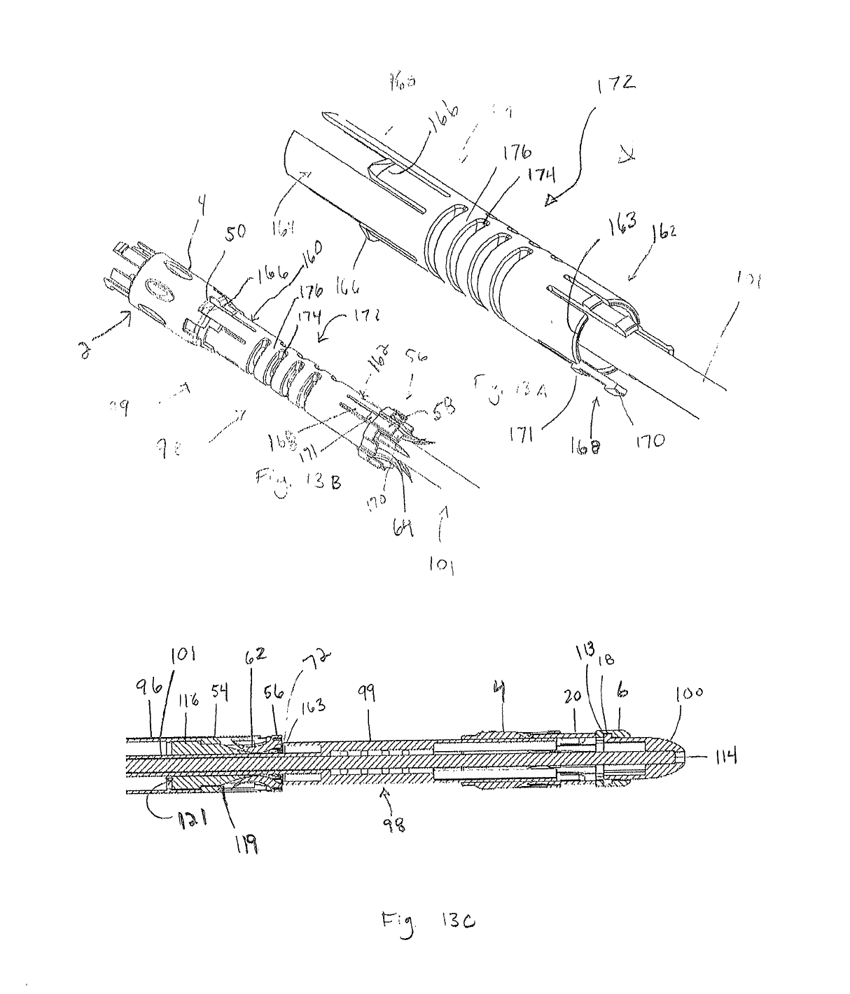

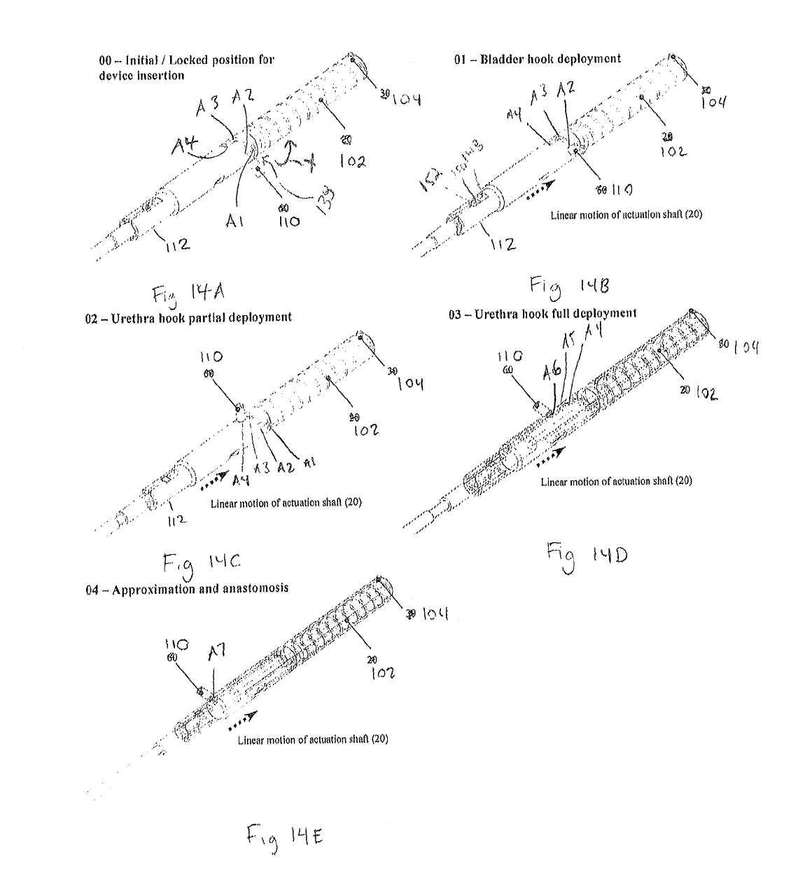

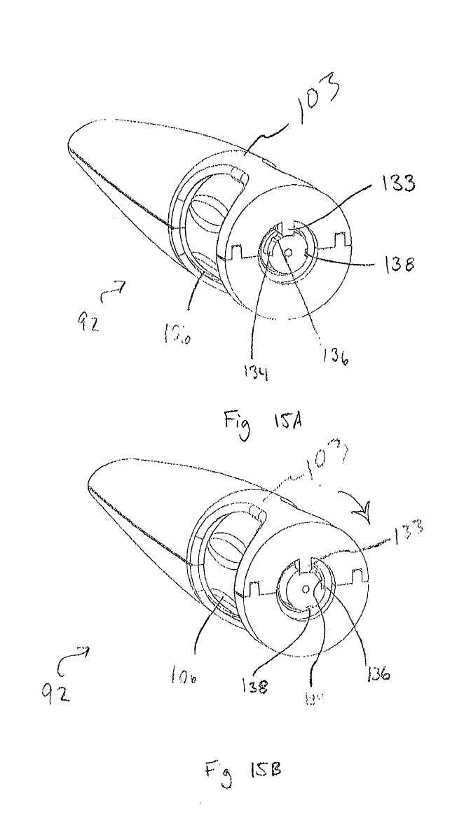

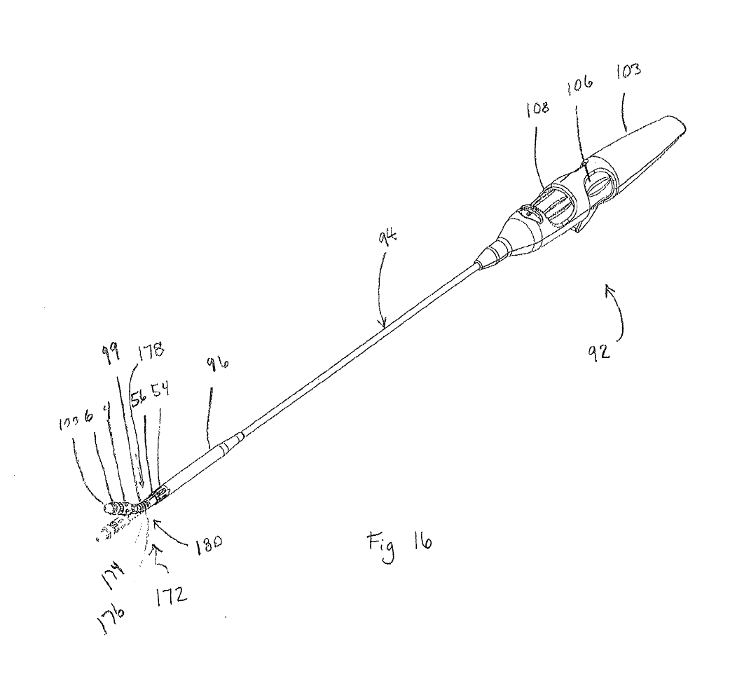

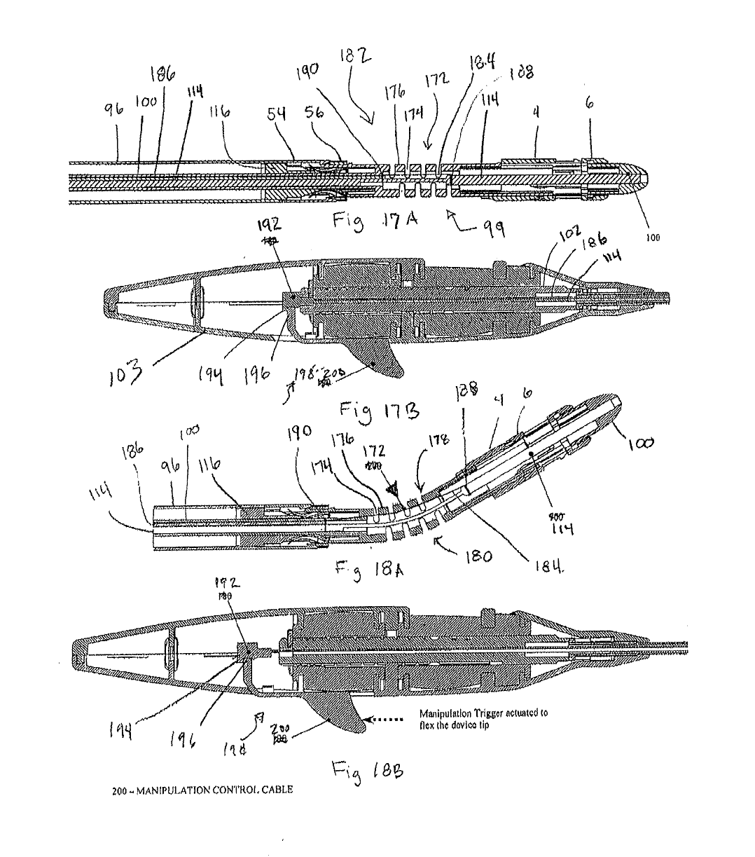

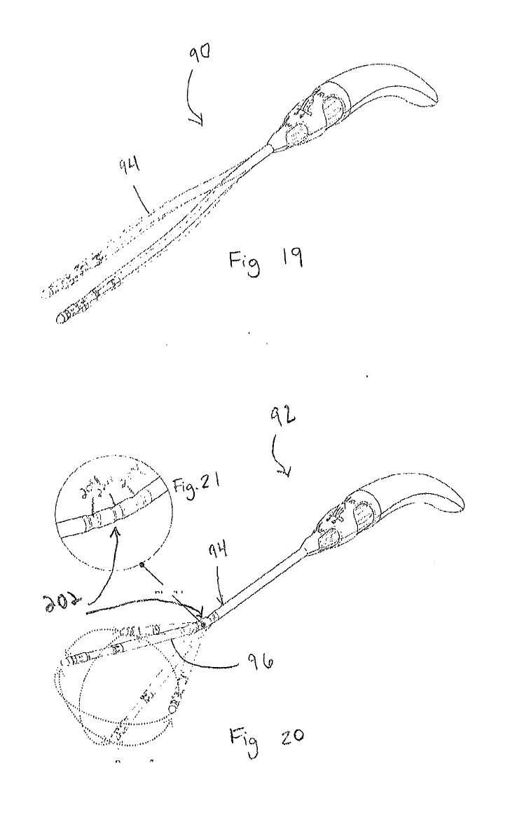

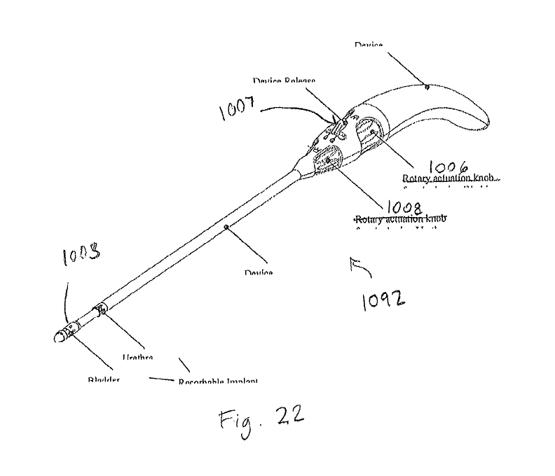

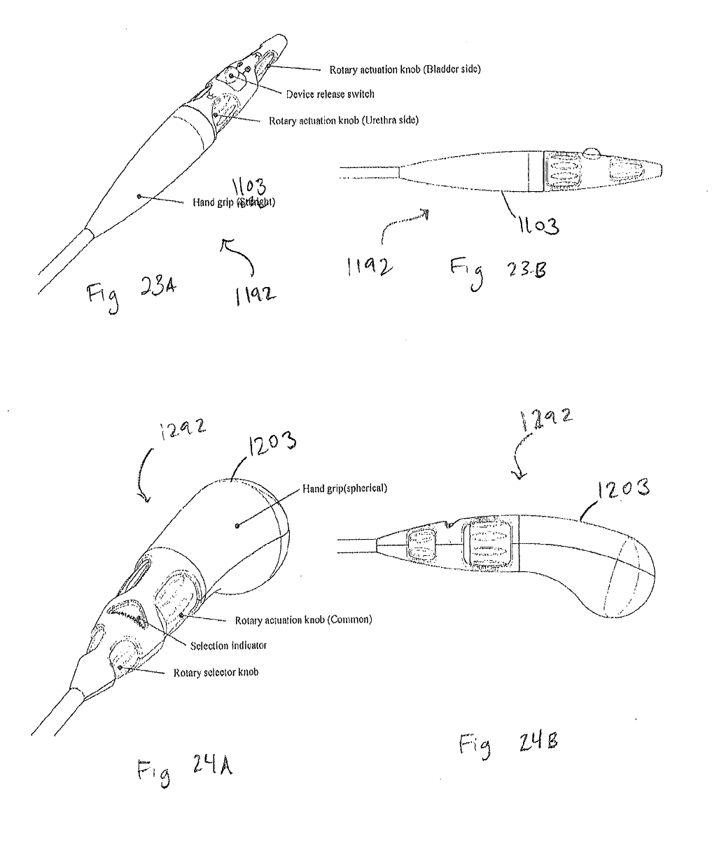

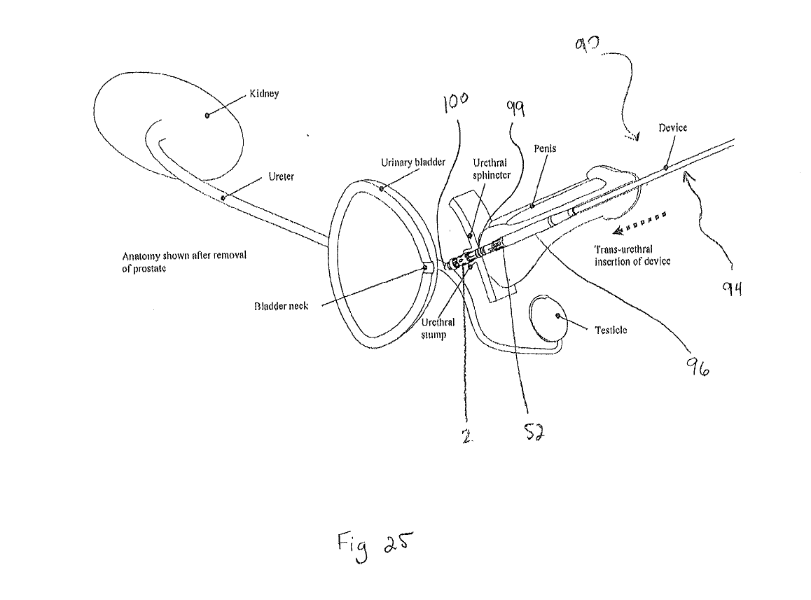

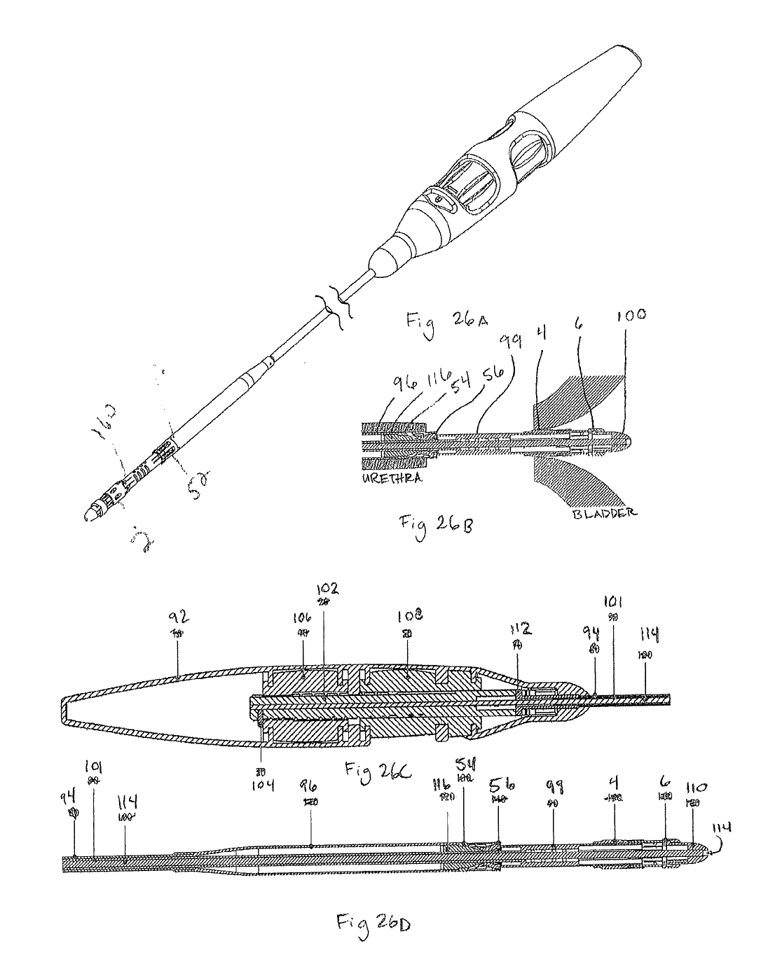

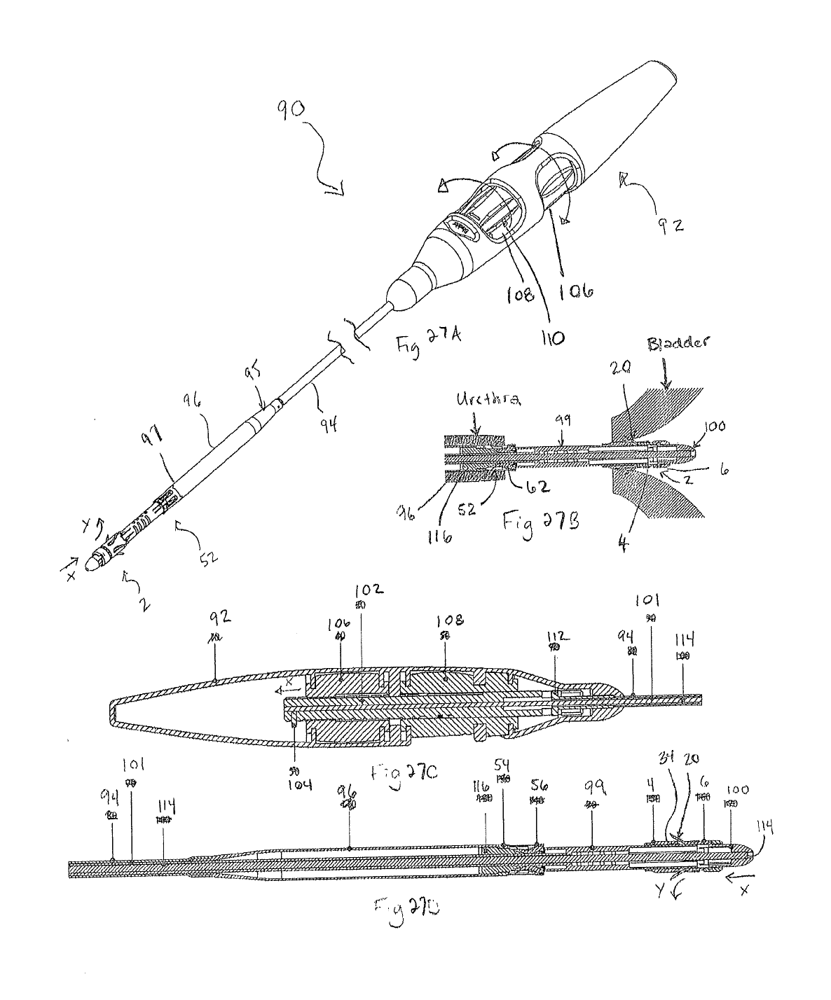

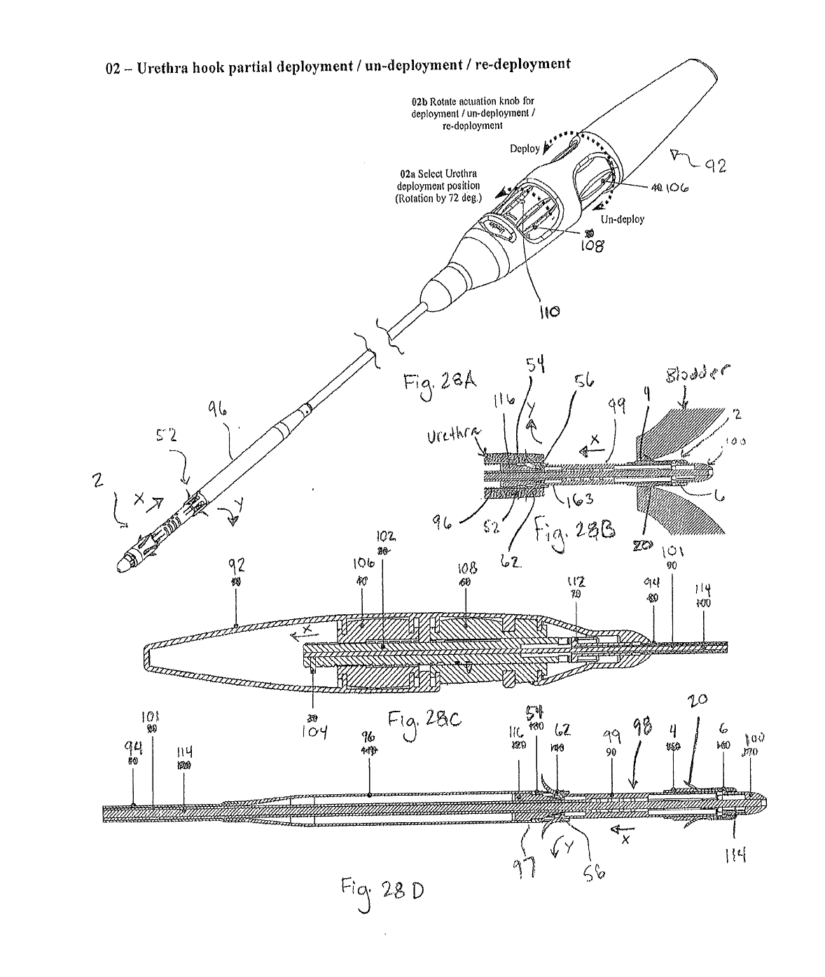

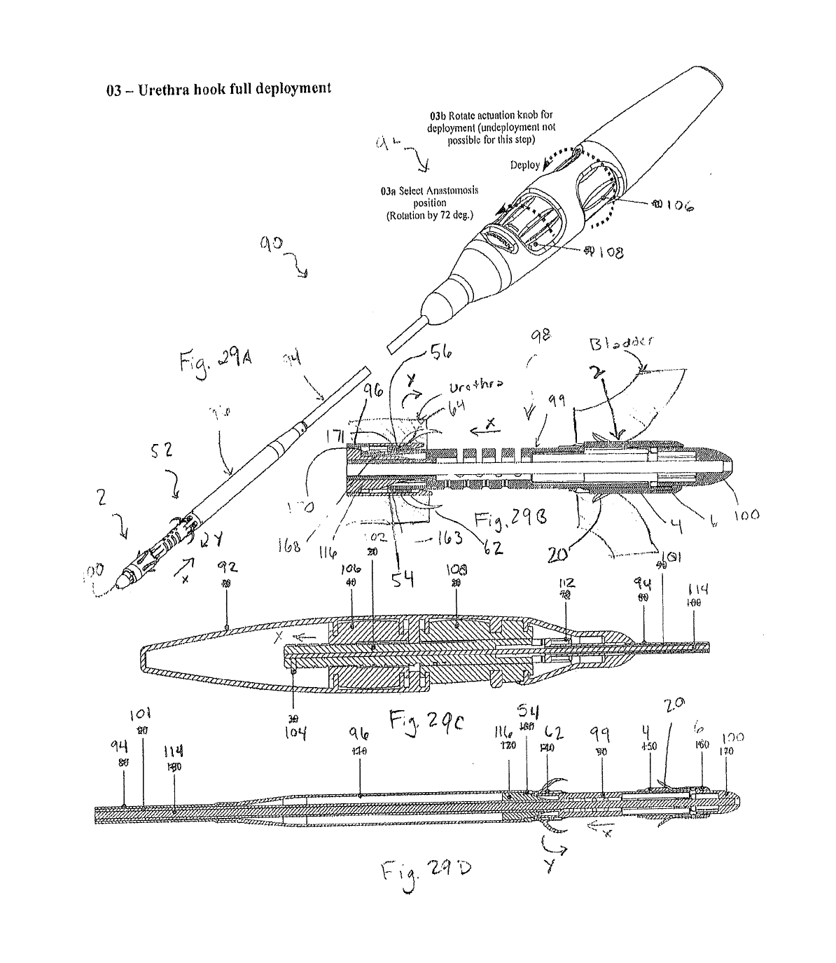

Turning now to the drawings, FIGS. 1 and 2 show a first ring assembly 2, which may be depicted as an upper or bladder ring assembly in certain applications of the device. In FIG. 1, the first ring assembly 2 is shown in the stored/retracted/delivery position. In FIG. 2, the first ring assembly 2 is shown in the deployed/extended position.

As shown in FIG. 1, the first ring assembly 2 comprises a first collar 4 and a first central ring 6. The first central ring 6 generally defines a ring shape having a first ring assembly wall 8 and lumen 10 that permits the passage of fluid therethrough. A distally facing surface 12 of the first ring assembly wall 8 defines locking tab receivers 14, which comprise indentations in the first ring assembly wall 8. The first ring assembly wall 8 facing the lumen 10 contains an axially extending device release groove 16 that communicates with a circumferentially extending deployment slot 18, along the interior of the first ring assembly wall 8. Additionally, the first central ring 6 has at least one first ring securement element 20, such as a tooth, extending axially in a proximal direction “P” from the first ring assembly wall 8 of the first central ring 6 opposite the distally facing surface 12. As shown, each first ring securement element 20 has an elongated body 22, a tissue piercing portion 24, and an inner surface 26. In FIG. 1, the elongated body 22 is generally straight, but may be curved so that the tissue piercing portions 24 are directed closer towards the lumen 10 of the first central ring 6.

In the depicted embodiment, the first ring securement elements 20 and the first central ring 6 are of a unitary construction. However, other constructions are possible. For example, the first ring securement elements 20 and the first central ring 6 may be separately constructed and the first ring securement elements 20 may each be pivotably mounted on the first central ring 6 so that the first central ring 6 forms a common axle for movement of the first ring securement elements 20 with respect to the first central ring 6.

As shown in FIG. 1, the first ring securement elements 20 are preferably formed from a resiliently flexible material that permits bending or flexing up to 30°, 90°, or 120° or any angle therebetween in a radial direction relative to the position shown in FIG. 1. The first ring securement elements 20 bend or flex from a stored/retracted/delivery position in which they extend axially from the first central ring 6 (as shown in FIG. 1) to a deployed/extended position in which they extend outward from the first collar 4 (as shown in FIG. 2) in order to engage and secure the first ring assembly 2 to tissue, such as the wall of the bladder neck or other hollow body part. Additionally, the first central ring 6 may be formed to include at least one living hinge (not shown) at a junction point 28 between at least one first ring securement element 20 and the first central ring 6. Alternatively, the deployment of the first ring securement elements 20 may rely on the flexibility and properties of the material forming the first ring securement elements 20 rather than a living hinge.

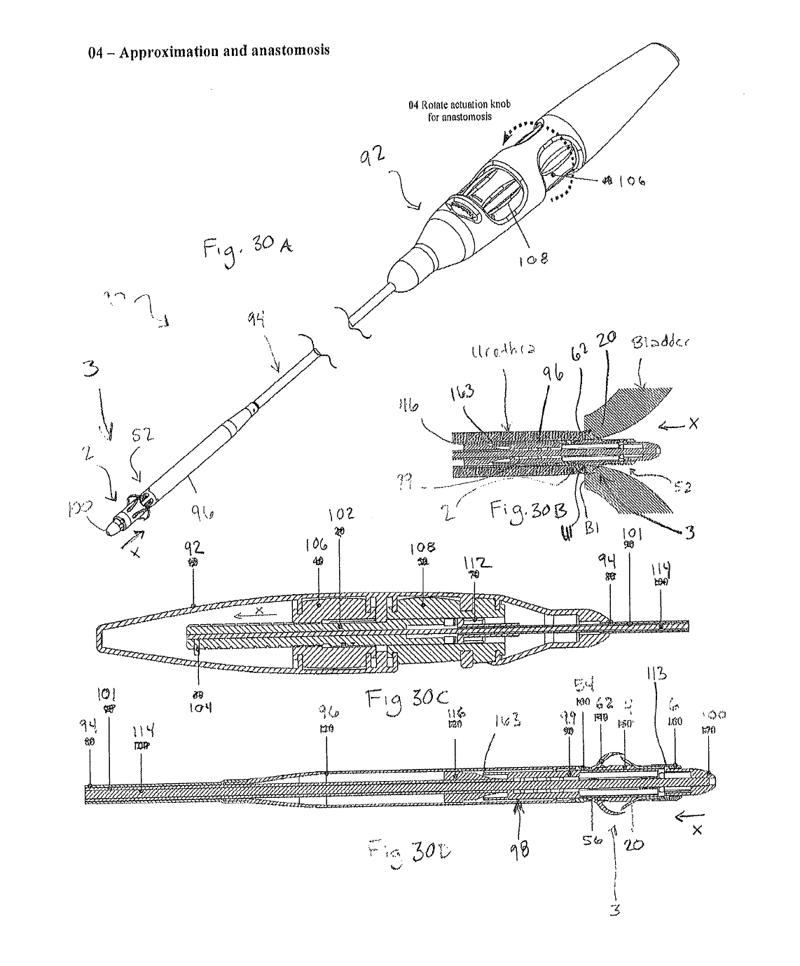

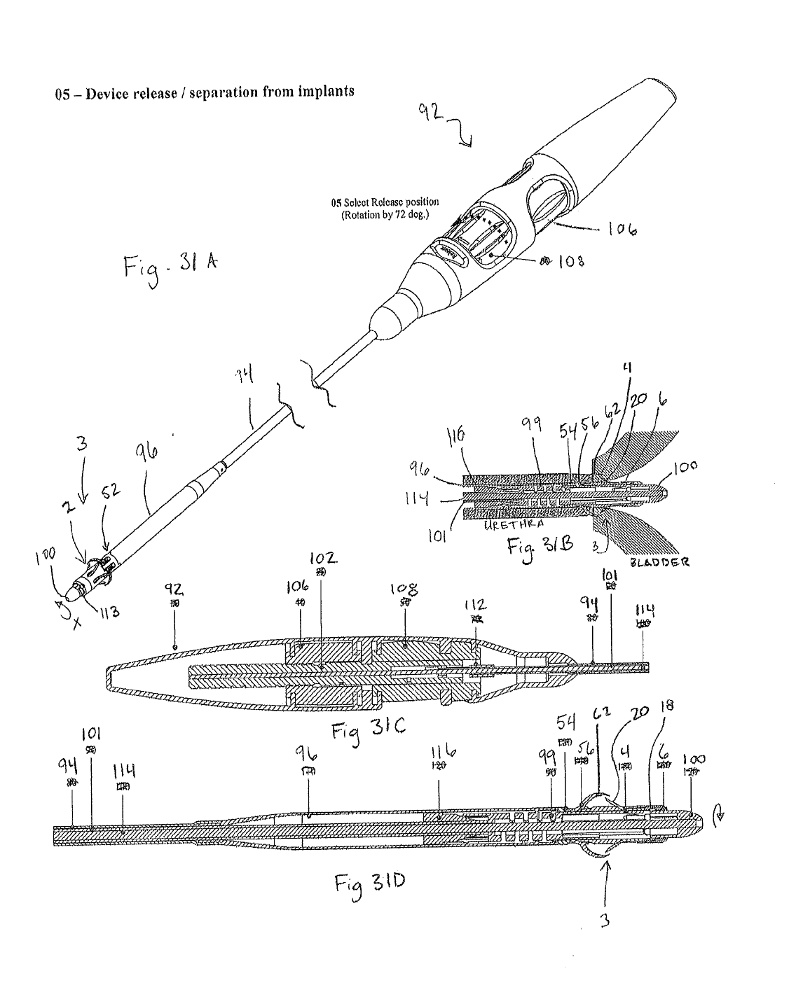

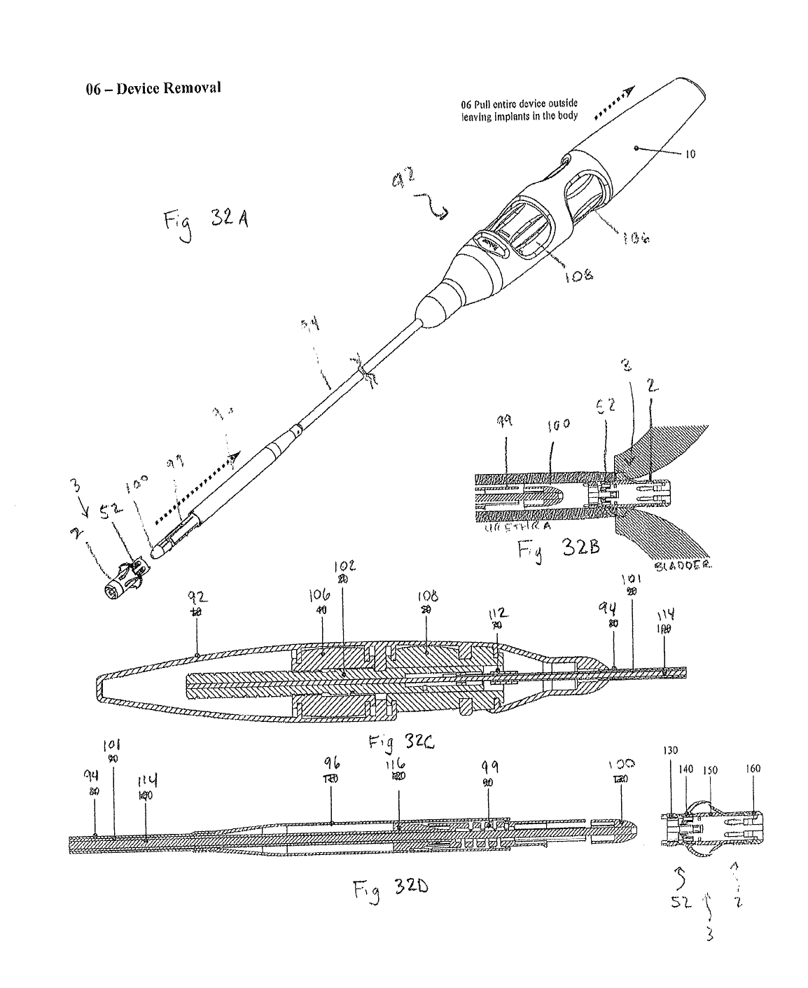

Referring to FIGS. 1 and 3, the first collar 4 is defined by a circumferential sidewall 30 comprising at least one axial groove 32 on its inner surface and at least one guide structure 34 in the sidewall 30. The first collar 4, defines a lumen 35 extending therethrough, which permits the passage of fluid through the first collar 4 and co-axially aligns with lumen 10 of the first central ring 6, when the first central ring 6 is mounted on the first collar 4. The axial grooves 32 extend axially along the interior surface of the circumferential sidewall 30 and are sized and shaped to guideingly receive a first ring securement element 20. The number and positioning of the axial grooves 32 correspond to the number and positioning of the first ring securement elements 20 such that each axial groove 32 may receive one first ring securement element 20.

The guide structures 34 are positioned in alignment with and proximally to the axial grooves 32. As shown in FIG. 1, the guide structures 34 define apertures 36 extending through the circumferential sidewall 30 of the first collar 4 that may extend at a proximally orientated angle with respect to the circumferential sidewall 30 of the first collar 4. The openings 36 of the guide structures 34 are sized and positioned to permit passage of the first ring securement elements 20 therethrough.

Still referring to FIGS. 1 and 3, each guide structure 34 defines an angled deployer surface 38 positioned to outwardly guide the first ring securement elements 20 as they pass through each aperture 36. When the first central ring 6 is mounted on the first collar 4, the first ring securement elements 20 extend through the internal lumen 6 of the first collar 4, into the axial grooves 32 and guide structures 34 such that a portion of the inner surfaces 26 of the first ring securement elements 20 engages the angled deployer surfaces 38. As shown in FIGS. 1 and 3, the number and positioning of the guide structures 34 correspond to the number and positioning of the first ring securement elements 20 such that each guide structure 34 may receive one first ring securement element 20.

Referring now to FIGS. 1 and 2, the first collar 4 further includes at least one ring mounting member 40 extending distally and axially from the first collar 4. Ring mounting members 40 include a ring wall receiving member 42 and a ring locking tab 44. The ring wall receiving member 42 is sized and configured to pass though the lumen 10 of the first central ring 6 and permit the first ring assembly wall 8 to be positioned between the circumferential sidewall 30 of the first collar 4 and the ring locking tab 44. As best seen in FIG. 2, when the first ring assembly wall 8 of the first central ring 6 is positioned between the circumferential sidewall 30 of the first collar 4 and a ring locking tab 44, (i) the ring locking tab 44 engages the locking tab receiver 14 of the first central ring 6 and (ii) the ring wall receiving member 42 is received in a extending device release groove 16. Engagement of the locking tab receivers 14 by the ring locking tabs 44 may restrict axial movement of the first central ring 6 with respect to the first collar 4, thereby securing the first central ring 6 and the first collar 4 together. As seen in FIG. 2, when the first central ring 6 and the first collar 4 are joined together, the first ring securement elements 20 fully project radially outward through the sidewall 30 of the first collar 4.

As best seen in FIG. 1, the first collar 4 also includes at least one ring guide 46 extending distally and axially from the circumferential sidewall 30 of the first collar 4. The ring guide 46 is a generally rectangular extension that may be received in the lumen 10 of the first central ring 6 to guide the mounting of the first central ring 6 onto the first collar 4. The ring guide 46 may be received within a groove or channel (not shown) in the first central ring 6 to guide mounting of the first central ring 6 onto the first collar 4. When the ring guide 46 is received in the groove or channel (not shown), the first ring securement elements 20 are aligned with guide structures 36 of the first collar 4 and rotational movement of the first central ring 6 with respect to the first collar 4 is restricted.

Turning now to the alternative view of the first collar 4 shown in FIG. 3, the first collar 4 is shown further including at least one first ring interconnecting element 47 proximally positioned on the first collar 4 for coupling the first collar 4 to the second collar 56 (shown in FIG. 6). The first ring interconnecting elements 47 can be provided as snap-fit connectors, screw-together connectors, adhesives or other conventional connector assemblies, whether detachable for decoupling or intended for one-time connection only. In typical embodiments, the first ring interconnecting elements 47 are provided by releasably interlocking catch surfaces that engage corresponding resiliently deflectable arms (such as second ring interconnecting elements 84 as depicted in FIG. 6), detents, push-pin assemblies, or other types of connectors for coupling two structures together.

In some examples, the first and second ring interconnecting elements 47, 84 may be configured to allow the ring assemblies 2, 52 to be selectively spaced apart from one another during coupling, for example, to accommodate variable length of the anastomosis or elasticity of the hollow body parts. For example, either or both of the first and second ring interconnecting elements 47, 84 may be provided with a plurality of notches, protuberances, or other coupling structures or means for coupling parts together (not shown in FIG. 2) that engage the opposing ring assembly to couple the first and second ring assemblies 2, 52 together. An example can be seen in FIG. 6A, where a second ring interconnecting element 84 includes multiple notches 84a for graduated attachment with the first ring assembly 2, via the first ring interconnecting element 47. Those skilled in the art will recognize that similar structures may also be provided on the first ring interconnecting assembly 47.

Another embodiment of ratcheting features that can be included on the first ring assembly 2 and second ring assembly 52 that are capable of providing a variable coupling distance between the first and second ring assemblies 2, 52 can be seen in FIG. 6B. As shown in FIG. 6B, the first ring assembly may include a plurality of interconnecting elements 47a that include a plurality of structures 47b that matingly engage corresponding interconnecting elements 47c included on the second ring assembly. Thus, in the embodiment shown in FIG. 6B, it is possible to couple the first and second ring assemblies 2, 52 together at three different distances. Thus, in the shown embodiment, the ring assemblies can be moved into contact with each other until the proximal-most structures 47b on the first ring assembly interconnecting elements 47a matingly engage the distal-most interconnecting elements 47c on the second ring assembly 52. Thus, in this position, the first and second ring assemblies 2, 52 are coupled together their farthest distance. If the surgeon desires to have a shorter coupling distance between the first and second ring assemblies 2, 52, the first and second ring assemblies 2, 52 may be moved closer together until the next-most structures 47b on the first ring assembly interconnecting elements 47a matingly engage the next-most interconnecting elements 47c on the second ring assembly 52. This process can continue until the desired coupling distance is achieved. In the depicted embodiment, the ratcheting features may be raised structures, detents, openings or any other structures that matingly engage each other to couple the first and second ring assemblies 2, 52 together. Although the depicted embodiment shows raised structures 47b on the first ring assembly interconnecting elements 47a and openings 47c in the second ring assembly 52 to receive the raised structures 47b, it is to be understood that the inclusion of these structures on the first and second ring assemblies 2, 52 may be reversed, i.e., the raised structures can be included on second ring assembly interconnecting elements.

The surgeon can manipulate the first and second ring assemblies 2, 52 so that a first notch or protuberance (not shown) or other similar structure on either or both the first and second ring interconnecting elements 47, 84 engages corresponding structures on the opposing ring assembly to couple the first ring assembly 2 at a first distance from the second ring assembly 52. If the first distance between the ring assemblies 2, 52 is determined to be too close or too far, the surgeon can manipulate the first and second ring assemblies 2, 52 so that a different notch or protuberance (not shown) or other similar structure on either or both the first and second ring interconnecting elements 47, 84 engages a corresponding structure on the opposing ring assembly to couple the first ring assembly 2 at a second distance from the second ring assembly 52. Those skilled in the art will recognize that adjusting the distance between the first and second ring assemblies 2, 52 can be performed numerous times until the desired distance between the two ring assemblies and hence, the desired magnitude of contact between the body tissue to be joined or connected, is obtained.

The first collar 4 further includes at least one proximally and axially extending second ring securement element locking member 48 for locking the second ring securement elements 62 of the second ring assembly 52 (shown in FIGS. 6 and 6C) in the deployed position when the first ring assembly 2 and second ring assembly 52 are coupled together (discussed in further detail with respect to FIGS. 6 and 6C). As shown, the second ring securement element locking member 48 extends proximally from the circumferential sidewall 30 of the first collar 4 adjacent to the support surfaces 50. The second ring securement element locking members 48 are preferably tapered from a thinner portion at its tip towards its thickest portion adjacent to the upper collar sidewall 30 to further assist in guiding the alignment and coupling of the ring assemblies 2, 52 together. There may also be additional taper provided to the side of each second ring securement element locking members 48 to help align the first and second ring assemblies about their longitudinal axis, if necessary. The second ring securement element locking member 48 serves to restrict rotation of first and second ring assemblies 2, 52 with respect to each other when the ring assemblies 2, 52 are coupled together, but preferably does not restrict axial movement. Instead, the lower ring interconnecting element 47 may help to limit unintended axial movement of the first ring assembly 2 with respect to the second ring assembly 52. The support surfaces 50 are proximally facing surfaces extending generally perpendicular to circumferential sidewall 30 of the first collar 4. As discussed further with respect to FIG. 13B, the support surfaces 50 facilitate the mounting of the first collar 4 for deployment.

Referring now to FIGS. 4 and 5, the first central ring 6 is mounted on the first collar 4 with the first ring assembly 2 in the retracted/stored position (FIG. 4) and the extended/deployed position (FIG. 5). As shown in FIG. 4, when the first ring assembly 2 is in the retracted or undeployed position, the first central ring 6 is spaced distally with respect to the first collar 4 such that the first ring securement elements 20 are received in axial grooves 32 and openings 36 and the tissue piercing portions 24 are directed towards the angled deployer surface 38. In this position, the first ring securement elements 20 are received by the first collar 4 such that the first ring securement elements 20 extend axially from the first central ring 6 in the proximal direction without substantially bending or flexing. Thus, in this position, the tissue piercing portions 24 do not engage body tissue.

FIG. 5 shows that movement of the first central ring 6 towards the first collar 4 during deployment urges the tissue piercing portions 24 and inner surfaces 26 of the first ring securement elements 20 against the angled deployer surfaces 38 of the first collar 4. Further translation or movement of the first central ring 6 towards the first collar 4 or vice versa, translation or movement of the first collar 4 towards first central ring 6, urges the first ring securement element body 22 to bend or flex where the first ring securement element 20 contacts the angled deployer surface 38 such that the first ring securement element 20 extends proximally and radially outward from the first collar 4 (as illustrated by arrow “x” in FIG. 5). Additionally, during translation or movement of the first central ring 6 towards the first collar 4 or vice versa, translation or movement of the first collar 4 towards first central ring 6, the ring mounting member 40 and the ring guide 46 may extend into the lumen 10 of the first central ring 6 and engage the inner surface of the first ring assembly wall 8. Where translation or movement of the first central ring 6 towards the first collar 4 or, vice versa, translation or movement of the first collar 4 towards first central ring 6, brings the first ring assembly wall 8 into contact with the circumferential sidewall 30 of the first collar 4, the ring locking tab 44 may engage the locking tab receiver 14 (as best seen in FIGS. 1 and 2). Engagement of the ring locking tab 44 with the locking tab receiver 14 may assist in restricting translational and/or rotational movement of the first central ring 6 with respect to the first collar 4, thus retaining the first ring securement elements 20 in the deployed position and also joining the upper collar 4 and upper central ring 6 together.

Turning now to FIGS. 6 and 6C, an exemplary second (e.g., lower or urethra) ring assembly 52 having a second collar 54 and a second central ring 56 is shown. The second central ring 56 has a second ring assembly wall 58 generally defining a lumen 60 extending therethrough, which permits the passage of fluid through the second central ring 56. At least one second ring securement element 62 is mounted on a second ring securement element mounting member 64 that defines a radially extending portion of the second ring assembly wall 58. Each of the second ring securement elements 62 extend axially along the lumen 60 of the second central ring 56. As shown, each second ring securement element 62 has a curved body 66, a tissue piercing portion 68, and an inner surface 70. In alternate embodiments, the second ring securement elements 62 may have a straight body. The second ring securement elements 62 also have a second ring securement element cam surface 72 opposite the piercing tip 68 and a pivot point 74.

As shown, the second ring securement elements 62 and the second central ring 56 are made of a unitary construction. The second ring securement elements 62 are adapted to bend, flex or rotate about a pivot point 74 from a stored/retracted/delivery position, in which they extend axially from the second central ring 56 through the lumen 60 (as shown in FIG. 6) to a deployed/extended position, in which they extend outward from the second central ring 56 (as best shown in FIGS. 6C, 29D, 30D, 31D, 41 and 42), such that the second ring securement elements 62 engage and secure the second ring assembly 52 to body tissue, such as the wall of the urethra neck or other hollow body part. In some examples, the pivot point 74 may comprise a living hinge; however, other structures are possible. For example, the second ring securement elements 62 and the second central ring 56 may be separately constructed and the second ring securement elements 62 may each be pivotably mounted on the second central ring 56 so that the second central ring 56 forms a common axle.

Still referring to FIG. 6, the second collar 54 is shown having a proximal ring base 76 and at least one longitudinally extending member 78 defining a lumen 80. The longitudinally extending members 78 extend axially and distally from the proximal ring base 76 and are spaced apart to slideably receive a second ring securement element mounting member 64 therebetween. Between each longitudinally extending member 78 is a distally facing surface of the proximal ring base 76 which defines an angled second ring securement element engagement surface 82. The second ring securement element engagement surface 82 is angled to engage the inner surface 70 of the second ring securement element 62 and deflect the second ring securement elements 62 outwards when the second central ring 56 is translated or moved towards the second collar 54 or, vice versa, the second collar 54 is translated or moved towards the second central ring 56.

As shown in FIG. 6, a second ring interconnecting element 84 is positioned distally on at least one of the longitudinally extending members 78 opposite the proximal ring base 76. The second ring interconnecting element 84 defines a protrusion extending into the lumen 80 and is configured to engage the first ring interconnecting element 47 and couple the second ring assembly 52 and first ring assembly 2 together when the second ring assembly 52 and first ring assembly 2 are urged towards mutual contact, as best seen, for example, in FIGS. 29D, 30D, 31D, and 42. The second ring interconnecting elements 84 can be snap-fit connectors, screw-together connectors, adhesives, or other conventional connector assemblies, whether detachable for decoupling or intended for one-time connection only. Additionally, a second central ring lock 86 is positioned distally on a shorter longitudinally extending member 87. The second central ring lock 86 includes a protrusion extending into the lumen 80 and is configured to engage the second central ring 56 when the second central ring 56 is received in the second collar 54, thereby allowing the second central ring 56 to be retained proximally of the first ring assembly 2, when the ring assemblies 2, 52 are deployed and attached to each other. A plurality of second central ring locks 86 and shorter longitudinally extending members 87 may be included. Alternatively, the second central ring 56 may be held in place within the second collar 54 by a friction fit. In any event, once the first ring assembly 2 and second ring assembly 52 are coupled together, this coupling will lock the second central ring 56 in place within the second collar 54.

Similar to the disclosure above with respect to FIG. 6A, the second central ring lock 86 may be provided with one or more notches (not shown) or similar structures that allow the surgeon to selectively couple the first ring assembly 2 with more or less proximity to the second ring assembly 52. Thus, the one or more notches or similar structures may serve as a ratcheting mechanism (not shown) that allows the surgeon to adjust the proximity of the first and second ring assemblies 2, 52 to accommodate the length or elasticity of the hollow body parts. Additionally or alternatively, the ratcheting mechanism (not shown) may be provided by one or more notches or similar structures provided on the first ring interconnecting element 47. Those skilled in the art will recognize that adjusting the distance between the first and second ring assemblies 2, 52 can be performed numerous times until the desired distance between the two ring assemblies and hence, the desired magnitude of contact between the body tissue to be joined or connected, is obtained.

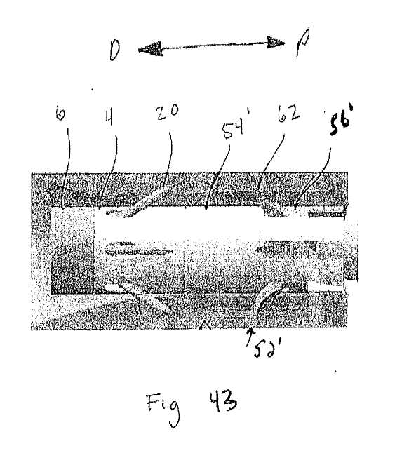

Referring to FIG. 39, the second collar 54 is configured to receive the second central ring 56 when the second central ring 56 is translated or moved towards the second collar 54, or vice versa, the second collar 54 is translated or moved towards the second central ring 56, such that the second ring securement element mounting members 64 and second ring securement elements 62 slide between adjacent extending members 78. As shown in FIG. 40, when the second central ring 56 slides proximally towards the proximal ring base 76 and past the second central ring lock 86, the second central ring lock 86 restricts translation of the second central ring 56 away from the second collar 54. Further advancement of the second central ring 56 into sliding engagement with the second collar 54 results in engagement of the inner surfaces 70 of the second ring securement elements 62 with the angled second ring securement element engagement surfaces 82 of the second collar 54. Engagement of the second ring securement elements 62 with the angled second ring securement element engagement surface 82 displaces the second ring securement elements 62 outwardly from the longitudinal axis of the second central ring 56, thereby urging the second ring securement elements 62 to pivot around a pivot point 74 and extend outward towards the partially deployed position.

As shown in FIG. 40, in the partially deployed position (as best seen in FIG. 40), the tissue piercing portions 68 of the second ring securement elements 62 extend outward in a generally proximal direction to pierce and engage the second hollow body part, such as the urethra. However, in the partially deployed position, the second ring securement elements 62 may not securely engage the second hollow body part so as to substantially restrict distal translation of the second central ring 56 with respect to the second hollow body part.

Furthermore, in the partially deployed position, a portion of the second ring securement element cam surface 72 extends into the lumens 60, 80 of the second central ring 56 and second collar 54. Additional force in the proximal direction applied to the second ring securement element cam surface 72 of the second ring securement elements 62 drives the second ring securement elements 62 towards full deployment (also shown in FIGS. 29A, 29B, 41, and 42). The second ring securement elements 62 pivot around a pivot point 74 from the undeployed position, such that the second ring securement element cam surfaces 72 are substantially axially aligned with the second ring securement element mounting member 64. In the fully deployed position (as shown in FIGS. 29D, 30D, 31D, 41, and 42), the second ring securement elements 62 may extend outward in a generally lateral direction and securely engage body tissue or a vessel such as the urethra, so as to substantially restrict translation or movement of the second ring assembly 52 with respect to the second hollow body part (e.g., urethra). Additionally, the tissue piercing portions 68 of the second ring securement elements 62 may be directed towards the second collar 54, as opposed to being pointed radially outward, into the surrounding tissue, thus minimizing damage to the surrounding tissue when the ring assembly 3 is in place.

Referring now to FIGS. 6C and 42, when the second ring assembly 52 and first ring assembly 2 are both fully deployed and brought into interlocking engagement, the second ring securement element cam surfaces 72 cooperate with the second ring securement element locking members 48 of the first collar 4 to lock the second ring securement elements 62 in the fully deployed position. When the second ring assembly 52 and first ring assembly 2 are urged towards interlocking engagement, the first ring assembly 2 and second ring assembly 52 are in axial alignment such that the second ring securement element locking members 48 of the first collar 4 extend into the lumen 60 of the second central ring 56. During coupling of the first ring assembly 2 and second ring assembly 52, the second ring securement element locking member 48 slide against the lumen-facing surface of the second ring securement element mounting members 64 and the second ring securement element cam surfaces 72 (which are axially aligned with the second ring securement element mounting members 64 in full deployment). The positioning of the second ring securement element locking member 48 within the lumen 60 and in contact with the second ring securement element cam surfaces 72 restricts movement of the second ring securement element cam surfaces 72 into the lumen 60, thereby locking the second ring securement elements 62 in the fully deployed position as shown in FIG. 6C.

Referring now to FIG. 6, at least one instrument engaging element 88 is provided on the second collar 54. The instrument engaging element 88 is a protrusion extending proximally from the proximal ring base 76 of the second collar 54 that engages an instrument 90 (shown in FIGS. 39-41) by friction fit, press fit, compression fit, or other attaching means. The instrument engaging element 88 restricts rotation of the second ring assembly 52 with respect to the insertion instrument 90 and proximal translation of the second collar 54 with respect to the insertion instrument 90. However, the instrument engaging element 88 is adapted to facilitate release of the second collar 54 from the insertion instrument 90 when the second ring assembly 52 is secured to the second hollow body part (e.g., urethra) and the insertion instrument 90 is translated proximally away from the second ring assembly 52.

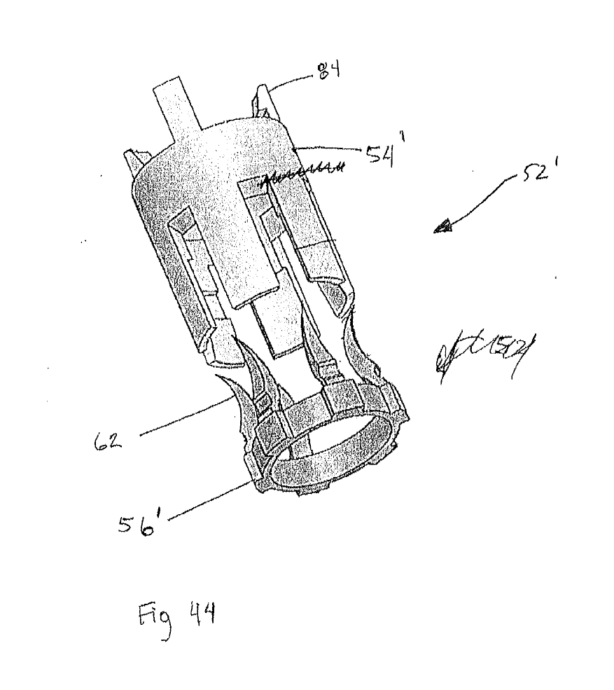

Referring now to FIGS. 43 and 44, a slightly modified alternative embodiment of the deployment of the second ring assembly 52′ is shown. As shown in FIG. 43, the second central ring 56′ may be mounted adjacent to the second collar 54′ on an opposite side of the second collar 54′ than the embodiment shown in FIG. 6. In the embodiment shown in FIG. 43, the second ring assembly 52′ may be deployed by translation or movement of the second central ring 56′ distally towards the second collar 54′. Additionally, as shown best in FIG. 44, an embodiment of a second ring assembly 52′ having the second central ring 56′ may be mounted proximally with respect to the second collar 54′ and may also be provided with second ring interconnecting elements 84 positioned distally on the second collar 54′.

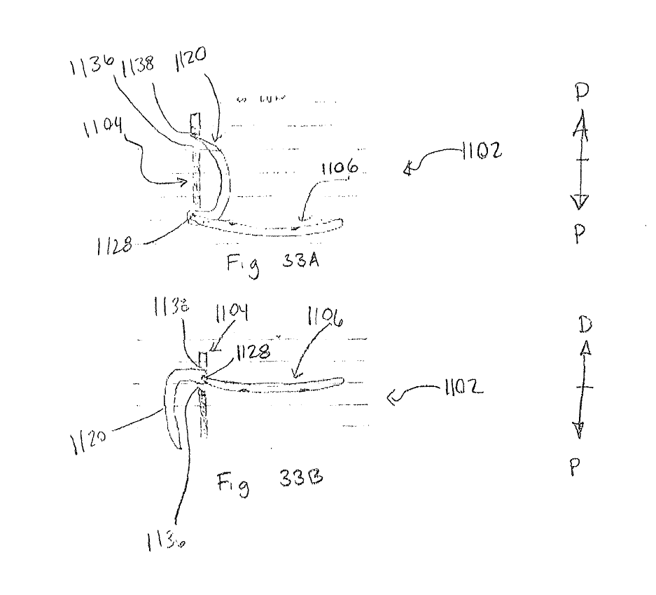

One skilled in the art will appreciate that alternate embodiments of a ring assembly 3 are possible, such as the alternative exemplary embodiment of a first ring assembly 1102 depicted in FIGS. 33A and 33B. Like the embodiment of a first ring assembly 2 shown in FIG. 1, the first ring assembly 1102 includes a first collar 1104 and a first central ring 1106. As shown, the first central ring 1106 may be of a unitary construction with the first ring securement elements 1120, and the first ring securement elements 1120 may be mounted on the first central ring 1106. Although a single first ring securement element 1120 is shown here for illustrative purposes, multiple first ring securement elements 1120 may be mounted to the same first central ring 1106. Unlike the embodiment of the first central ring 6 shown in FIG. 1, the first central ring 1106 shown in FIGS. 33A and 33B may be configured to rotate or evert during deployment of the first ring securement elements 1120.

As shown in FIGS. 33A and 33B, the distal translation or movement of the first central ring 1106, with respect to the first collar 1104, or vice versa, the proximal translation or movement of the first collar 1104 with respect to the first central ring 1106, urges the first ring securement elements 1120 into contact with the guide structures 1138 of the first collar 1104. The force of the first ring securement elements 1120 against the guide structures 1138 of the first collar 1104 urges the first ring securement elements 1120 to pivot at and translate through the rotation of the first central ring 1106 about itself. The first central ring 1106 is sufficiently flexible to allow eversion wherein an inner facing surface is positioned to face outwards and an outward facing surface is positioned to face inwards. Accordingly, the pivoting motion of the first ring securement elements 1120 causes the first central ring 1106 to also rotate and evert. As shown in FIGS. 33A and 33B, the dots on the first central ring 1106 rotate from an upward direction shown in FIG. 33A to a downward direction shown in FIG. 33B as the first central ring 1106 rotates and everts. Optionally, the first central ring 1106 may comprise living hinges 1128 used to mount the first ring securement elements 1120 and reduce the overall stress on the first ring securement elements 1120 by allowing the first central ring 1106 to rotate. As a result, the stress concentration at the living hinge 1128 is reduced, thus reducing the chance of failure at the living hinge during deployment. Additionally, there may be cam structures or ratcheting teeth (not shown) on the back of the securement elements. In preferred examples, a stop mechanism is a tooth (not shown) on the central ring 1106 that rotates 180 degrees within the collar 1104 and then abuts an internal structure on the inner wall of the collar 1104 to resist rotation of the first central ring 1106 back to the undeployed position. Additionally, one skilled in the art will appreciate that a structure similar to FIGS. 33A and 33B may be adapted for use a second ring assembly (not shown) for engagement and securement to the urethra or other hollow body part.

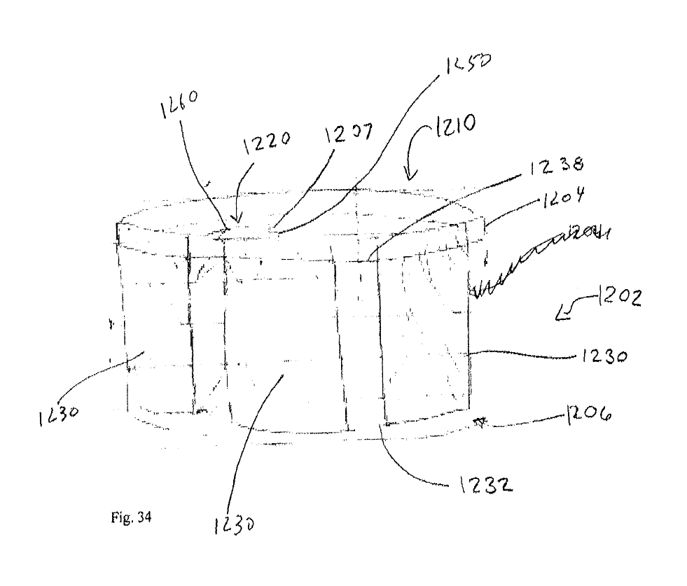

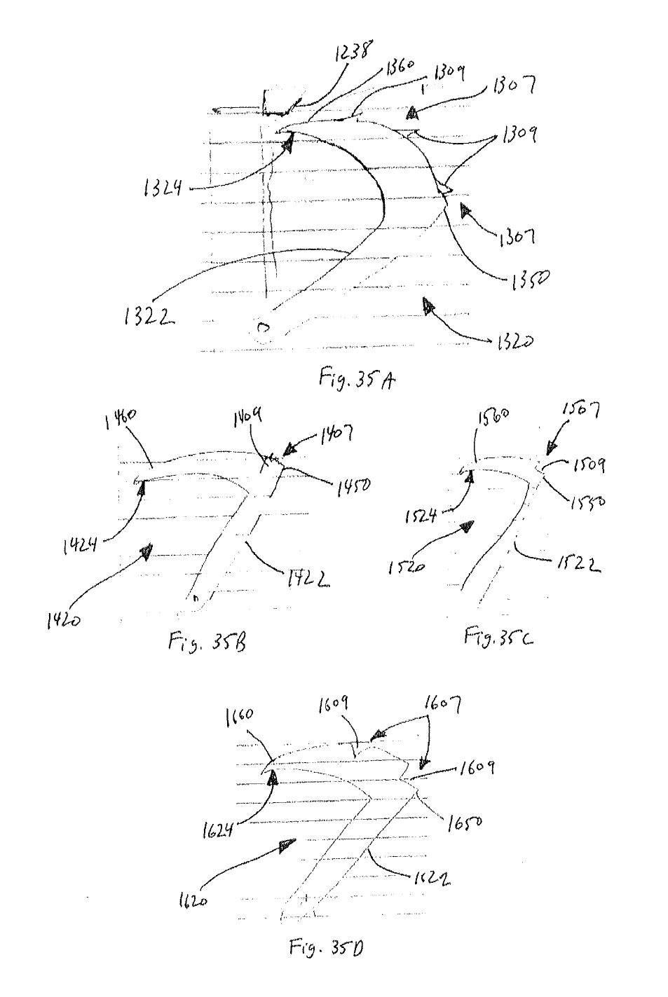

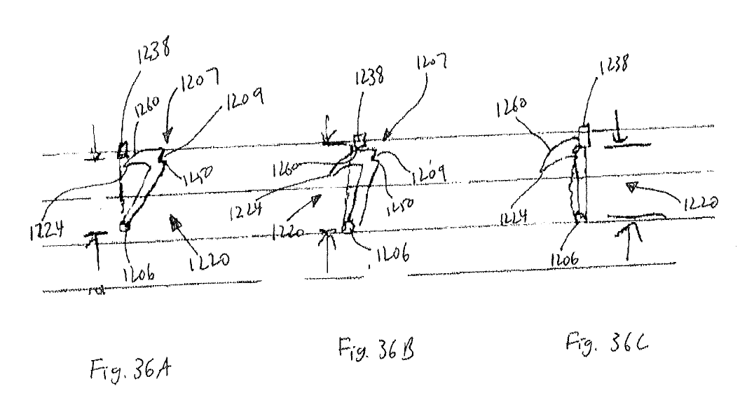

Additionally, a further alternative embodiment of a first ring assembly 1202 is depicted in FIG. 34. As shown, the first ring assembly 1202 is defined by a circumferential sidewall, which is made up of multiple panels 1230 that attach to a first ring structure 1204 and a second ring structure 1206, thereby defining the circumferential wall of the first ring assembly 1202. Preferably, the panels 1230 are formed from a flexible and elastic fabric, polymer sheeting, or other material so long as the material is flexible and elastic.

As also shown in FIG. 34, the panels 1230 are arranged about the circumference of the first ring assembly 1202 such that axially extending slots 1232 separate each panel. Each of the axially extending slots 1232 is sized and spaced to receive a first ring securement element 1220, which are pivotably mounted on the second ring structure 1206. The circumferential sidewall further defines guide surfaces 1238 positioned distally in the axially extending slots 1232 on the first ring structure 1204. In alternate embodiments, the circumferential sidewall may be made from a single flexible and elastic material attached to the first ring structure 1204 and second ring structure 1206. In such embodiments, the axially extending slots may be cut into the flexible and elastic material.

As shown in FIG. 34, the first ring securement elements 1220 may define at least one ratcheting element 1207 (or means for adjusting the positioning of the first ring securement elements 1220 with respect to the circumferential sidewall) positioned to engage the guide surface 1238 of the first ring structure 1204 during deployment of the first ring assembly 1202. As best seen in the exemplary embodiments of alternative first ring securement elements (1320, 1420, 1520, 1620) shown in FIGS. 35A-35D, the first ring securement elements 1320, 1420, 1520, 1620 may define a bent or sickle-shaped body 1322, 1422, 1522, 1622 with a curved tissue piercing portion 1224, 1324, 1424, 1524, 1624. As shown, the tissue piercing portion 1224, 1324, 1424, 1524, 1624 is provided with a ratcheting element 1207, 1307, 1407, 1507, 1607 in proximity to the piercing tip of the securement element. As shown in FIG. 35A, a ratcheting element 1307 may be defined by at least one tooth 1309 extending from the tissue piercing portion 1324 of the first ring securement element 1320. Alternatively, as shown in FIGS. 35B-35D, a ratcheting element 1407, 1507, 1607 may be defined by at least one notch 1409, 1509, 1609 in the tissue piercing portion 1424, 1524, 1624. In alternate embodiments, the first ring securement elements may include multiple teeth or notches.

Referring again to FIG. 34, when the panels 1230 are in the unflexed or unstressed state, the distance between the first and second ring structures 1204, 1206 and hence the height of the axially extending slots 1232, is less than the height of the first ring securement elements 1220 such that the first ring securement elements 1220 are prevented from extending through the slots 1232 and are, therefore, maintained within the diameter of the first ring assembly 1202. Thus, in order to deploy the first ring securement elements 1220 through the axially extending slots 1232 and into body tissue, portions of the insertion instrument are brought into contact with the interior surface 1250, 1350, 1450, 1550, 1650 of the securement elements 1220, 1320, 1420, 1520, 1620. Further pressure or force exerted by the insertion instrument on the interior surfaces 1250, 1350, 1450, 1550, 1650 of the securement elements 1220, 1320, 1420, 1520, 1620 in a direction away from the longitudinal axis of the first ring assembly 1202, forces the securement elements 1220, 1320, 1420, 1520, 1620 to move in a corresponding direction into the axially extending slots 1232 such that a top surface 1260, 1360, 1460, 1560, 1660 of the first securement elements 1220, 1320, 1420, 1520, 1620 acts on the first ring structure 1204. Because the panels 1230 are made from a flexible and elastic material, as the first securement elements 1220, 1320, 1420, 1520, 1620 are further forced into axially extending slots 1232 by the insertion instrument, the shape of the top surface 1260, 1360, 1460, 1560, 1660 of the first securement elements 1220, 1320, 1420, 1520, 1620 forces the first ring structure 1204 away from the second ring structure 1206 thereby increasing the distance between the first and second ring structures 1204, 1206 and hence the length or height of the axially extending slots 1232. The increased length or height of the axially extending slots 1232 permits the first securement elements 1220, 1320, 1420, 1520, 1620 to enter into and through the axially extending slots 1232. The insertion instrument may push the first securement elements 1220, 1320, 1420, 1520, 1620 outwardly causing them to extend through the axially extending slots and into body tissue until a tooth 1309 or a notch 1409, 1509, 1609 catches on the first ring structure 1204. Once a tooth 1309 or a notch 1409, 1509, 1609 catches on the first ring structure 1204, tension on the first ring structure as a result of the flexible and elastic material of the panels 1230 acts to lock the first securement elements 1220, 1320, 1420, 1520, 1620 in the deployed position.

Moreover, because the panels 1230 and hence the material that forms the sidewall are made from a flexible and elastic material, after the first securement elements 1220, 1320, 1420, 1520, 1620 are deployed and held in place by the interaction of the ratcheting elements 1207, 1307, 1407, 1507, 1607 with the first ring structure 1204, the distance between the first ring structure 1204 and second ring structure 1206 can be increased because of the ability of the flexible and elastic material to stretch. Once the distance between the first and second ring structures 1204, 1206 is increased a sufficient amount, the ratcheting elements 1207, 1307, 1407, 1507, 1607 will disengage from the first ring structure 1204 allowing the first securement elements 1220, 1320, 1420, 1520, 1620 to retract within the circumference of the first ring assembly 1202 thereby permitting the surgeon to reposition the first ring assembly 1202 within the body vessel. This process can be repeated multiple times until the first ring assembly 1202 is properly positioned.

As shown in FIGS. 35A and 35D, in alternate embodiments, the ratcheting element may include multiple teeth 1309 (FIG. 35A) or multiple notches 1609 (FIG. 35D) such that the first securement elements 1220, 1320, 1420, 1520, 1620 may be extended outwardly through the axially extending slots 1232 at differing degrees depending on how much body tissue penetration the surgeon desires.

FIGS. 36A-36C depict an exemplary deployment procedure for first ring securement elements 1320, 1420, 1520, 1620 being provided with a notch 1409, 1509, 1609 or a tooth 1309, where the notch 1409, 1509, 1609 or tooth 1309 engages the guide surface 1238 of the first ring structure 1204 when the first ring securement element 1320, 1420, 1520, 1620 pivots radially with respect to the second ring structure 1206. Engagement of the notch 1209 with the guide surface 1238 causes the ratcheting element 1207 to restrict further pivoting movement of the first ring securement element 1220 with respect to the second ring structure 1206. The ratcheting element 1207 can be released to allow further pivoting movement of the first ring securement elements 1220 with respect to the first central ring 1206 by stretching of the panels 1230 in distal and/or proximal directions. Release of the ratcheting element 1207 may permit the first ring securement elements 1220 to retract towards the undeployed position or, in embodiments having a ratcheting element 1207 with plurality of teeth 1209, to pivot outwards until the guide structure 1238 engages a second tooth 1209.

One skilled in the art will appreciate that the alternative embodiments of the first ring assembly 1102, shown in FIGS. 33A and 33B, and the first ring assembly 1202, shown in FIG. 34, can also be utilized in a second ring assembly (not shown) or be used interchangeably with the design for ring deployment shown in FIGS. 1-6. One skilled in the art will further appreciate that any of the above disclosed ring assemblies can be used or modified for use in engaging and securing tissue, such as either of the bladder and the urethra, or any other hollow body part.

Insertion Instrument

Turning now to FIGS. 7 and 8, an exemplary embodiment of an insertion instrument 90 is shown. The insertion instrument 90 may be used to (i) insert the second ring assembly 52 in a specific anastomosis site and the first ring assembly 2 into adjacent tissue, e.g. the bladder and urethra or other hollow body parts, (ii) separately deploy the respective securement elements 20, 62, and (iii) couple the second ring assembly 52 and the first ring assembly 2 together. The insertion instrument 90 can be withdrawn from the patient leaving the second ring assembly 52 and the first ring assembly 2 in place, sealing the anastomosis.

As shown in FIG. 7, the insertion instrument 90 includes a handle assembly 92, a tube 94 (which can be flexible or rigid but is preferably flexible), an outer housing 96, an implant support 98 and a deployer 100 located at the distal tip of the insertion instrument 90. The flexible tube 94 is a generally elongate tube. The outer housing 96 is tube-shaped with a flexible tube-engaging portion 95 that tapers into a circumference similar to that of the flexible tube 94 and a second collar mounting portion 97, having a circumference similar to that of the second collar 54. The implant support 98 defines a generally cylindrical distal implant mounting portion 99 and a generally elongate, tubular implant support shaft 101 extending proximally from the implant mounting portion 99 into the flexible tube 94 (seen best in FIG. 8). The deployer 100 is generally conical and is mounted distally on an elongate deployer shaft 114 (seen best in FIG. 8).

As shown in FIG. 7, when the insertion instrument 90 is assembled, the flexible tube 94 is disposed between the handle assembly 92 and the outer housing 96. The implant mounting portion 99 of the implant support 98 extends distally from the second collar mounting portion 97 of the outer housing 96. The deployer 100 extends distally from the implant mounting portion 99 of the implant support 98.

As best seen in FIG. 8, at least a portion of the flexible tube 94, implant support 98, and outer housing 96 respectively define lumens 117, 118 and 115 extending therethrough. The diameter of the lumen 117 within the flexible tube 94 and lumen 115 of the outer housing 96 are each sized to slideably receive a portion of the implant support shaft 101. Further, the diameter of the lumen 115 of the outer housing 96 is greater than the diameter of the implant mounting portion 99 of the implant support 98, such that the outer housing 96 can receive a portion of the implant mounting portion 99. The lumen 118 of the implant support 98 is sized to slideably receive a portion of the deployer shaft 114. Thus, when the implant support shaft 101 and deployer shaft 114 are received within the lumen 117 of the flexible tube 94, as the insertion instrument 90 is assembled, the flexible tube 94, implant support shaft 101, and deployer shaft 114 form coaxial elongate members. Due to this coaxial arrangement, the implant support shaft 101 and deployer shaft 114 can translate axially with respect to the handle assembly 92 within the lumens 117, 115 of the flexible tube 94 and outer housing 96.

Furthermore, as seen in FIG. 8, the implant support shaft 101 is of a length such that the implant mounting portion 99 can extend distally from the outer housing 96 while a portion of the implant support shaft 101 is received within the handle assembly 92 when the insertion instrument 90 is assembled. Similarly, the deployer shaft 114 is of a length such that the deployer 100 can extend distally from the implant mounting portion 99 when the insertion instrument 90 is assembled while a portion of the deployer shaft 114 is proximally received within the handle assembly 92.

As seen in FIG. 8, a urethra side cam 116, which defines a cone shape with a lumen 121 and a tapered portion 119, is slideably mounted in the second collar mounting portion 97 of the outer housing 96. The tapered portion 119 of the urethra side cam 116 extends distally from the second collar mounting portion 97 of the outer housing 96. The lumen 121 of the urethra side cam 116 is sized to slideably receive the implant support shaft 101 and is in coaxial alignment with the outer housing 96 (as seen best in FIG. 13C). Thus, as best seen in FIG. 13C, in the assembled insertion instrument 90, the implant support shaft 101 can pass through the lumen 121 of the urethra side cam 116.

As shown in FIG. 7, when the anastomosis system 1 is assembled, the first ring assembly 2 and second ring assembly 52 are mounted in spaced relation to each other, on the distal portion of the insertion instrument 90. The second collar 54 engages the second collar mounting portion 97 of the outer housing 96, via the instrument engaging elements 88. The second central ring 56 is mounted proximally on the implant mounting portion 99 of the implant support 98 and positioned distally of the second collar 54, with the second ring securement elements 62 extending axially within the second collar 54 and the outer housing 96 (also seen in FIG. 13C). As best seen in FIG. 13C, the tapered portion 119 of the urethra side cam 116 extends into the lumen 80 of the second collar 54 and engages the inner surfaces 70 of the second securement elements 62. The first collar 4 is mounted distally on implant mounting portion 99 of the implant support 98. The first central ring 6 is mounted on the deployer 100 and positioned proximal of the first collar 4.

The second ring assembly 52 and first ring assembly 2 are mounted on the insertion instrument 90 such that the first ring interconnecting elements 47 are axially aligned with the second ring interconnecting elements 84 and the second central ring locks 86 are axially aligned with the support surfaces 50 of the first collar 4. In the embodiment shown, the first and second ring assemblies 2, 52 are not intended to rotate about their common longitudinal axis during deployment of the securement elements 24, 62 and attachment to each other. The second ring securement element locking members 48 are also axially aligned with the second ring securement element cam surfaces 72.

As shown in FIGS. 7, 8 and 11, the handle assembly 92 includes an actuation shaft 102, a hollow grip member 103, a stopper cross-pin 104, a rotary actuation knob 106 and a rotary selection knob 108. The rotary selection knob 108 includes an opening defining a plunger pin receiver 109 that is sized to receive a plunger pin 110. The handle assembly 92 further includes an adapter 112 that is mechanically coupled to the actuation shaft 102.

In general, the handle assembly 92 is assembled such that the stopper cross pin 104, pin rotary actuation knob 106, rotary selection knob 108, plunger pin 110 and adapter 112 are mounted on or in the actuation shaft 102. Additionally, the actuation shaft 102, stopper cross pin 104, pin rotary actuation knob 106, rotary selection knob 108, plunger pin 110, adapter 112 are mounted within a lumen 105 extending within the hollow grip member 103.

Turning now to FIGS. 9A and 9B, detailed views of the actuation shaft 102 and adapter 112 are shown. As pictured, the actuation shaft 102 has an internal lumen 122 defining a passageway through an elongated tubular body 124, with the passageway sized to receive a portion of the deployer shaft 114 and a portion of the adapter 112. When the insertion instrument 90 is assembled, the deployer shaft 114 is fixed within the lumen 122 of the actuation shaft 102 such that the axial or rotational motion of the actuation shaft 102 is transferred to the deployer shaft 114.

The outer surface of the tubular body 124 has a threaded portion 126 located adjacent the proximal end 128. The proximal end 128 of the actuation shaft 102 also defines a stopper cross-pin opening 130 for receiving the stopper cross-pin (as best seen in FIG. 11). Additionally, the actuation shaft 102 includes a device guide slot 132 extending distally from the proximal end 128 along the length of the threaded portion 126. The device guide slot 132 is sized to receive the hollow grip release detent 133 of the hollow grip member 103 (shown in FIGS. 15A and 15B) to permit axial sliding of the actuation shaft 102 with respect to the hollow grip member 103 during assembly and use of the insertion instrument 90. As shown in FIG. 9B, the device guide slot 132 terminates in a circumferential recess 134 that defines an outward extending actuation shaft detent 136. The actuation shaft detent 136 cooperates with the hollow grip release detent 133 of the hollow grip member 103 to provide an audible sound and physical indication that the insertion instrument 90 is set to the “Release” position (as best seen in FIGS. 15A and B).

As best seen in FIG. 9A, the actuation shaft 102 further includes a plunger guide 138 that defines a grooved and angled pathway. The angled pathway of the plunger guide 138 defines a series of right angles A1-A4 traced by the plunger guide 138 alternating between either extending: (1) counter-clockwise and perpendicular to a longitudinal axis 140 of the actuation shaft 102 (preferably at 72°); or (2) distally and parallel to the longitudinal axis 140 of the actuation shaft 102. The plunger guide 138 has a width adapted to receive a portion of the plunger pin 110 when the insertion instrument 90 is assembled. As discussed below in detail with respect to FIGS. 14A-14E, movement of the plunger pin 110 through the plunger guide 138 allows the rotary selection knob 108 to select the second ring assembly 52 or first ring assembly 2 for deployment or coupling.

Still referring to FIGS. 9A and 9B, the distal portion 142 of actuation shaft 102 includes longitudinally extending arms 144, which define an axially extending adaptor slot 146. The adaptor slot 146 terminates in an adaptor guide receiver 148 defining an aperture with a protruding adaptor detent 150.

Although the embodiment of an actuation shaft 102 shown in FIGS. 9A and 9B is of unitary construction, one skilled in the art will appreciate that an actuation shaft may be an assembly of two or more separate shafts (not shown). An actuation shaft formed from separate shafts may advantageously permit the independent deployment of the ring assemblies 2, 52.

As seen in FIGS. 10A-10C, the adaptor 112 is generally tubular with a lumen 151 defining a passageway therethrough and has an outwardly extending adaptor guide 152. The lumen 151 is sized to slideably receive the deployer shaft 114 and a portion of the implant support shaft 101. Furthermore, the portion of the implant support shaft 101 received within the lumen 151 is fixed to the adaptor 112 to restrict axial and rotational motion of the adaptor 112 with respect to the implant support shaft 101.

The adaptor 112 may be inserted into the lumen 122 by spreading the longitudinally extending arms 144 apart to allow the adaptor guide 152 to move through the adaptor slot 146 and into the adaptor guide receiver 148 proximal of the adaptor detent 150. When the adapter 112 is received in the lumen 122 of the actuation shaft 102, the adaptor guide receiver 148 is free to move proximally with respect to the adaptor guide 152 until the first ring securement elements 20 of the first ring assembly 2 are deployed. As shown in FIG. 10C, after the proximal translation of the actuation shaft 102 and adaptor guide receiver 148, the adaptor detent 150 engages the adaptor guide 152 to restrict both longitudinal and rotational motion of the adaptor 112 with respect to the actuation shaft 102. Thus, when the adaptor guide receiver 148 is engaged by the adaptor detent 150 (i.e., after deployment of the first ring assembly 2), axial translation of the actuation shaft 102 will carry the adaptor 112 (and the implant support shaft 101 mounted thereto) in a coordinating movement.

Additionally, as seen in FIG. 10A, the threaded portion 126 of the actuation shaft 102 passes through the rotary actuation knob 106. The rotary actuation knob 106 is provided with a threaded lumen 154 that matingly engages the threaded portion 126 of the actuation shaft 102. Thus, rotation of the rotary actuation knob 106 in the counter-clockwise direction with respect to the actuation shaft 102 causes the actuation shaft 102 to translate proximally with respect to the rotary actuation knob 106 (as shown by arrows x and y in FIG. 10A). Likewise, rotation of the rotary actuation knob 106 in the clockwise direction with respect to actuation shaft 102 causes the actuation shaft 102 to translate distally with respect to the rotary actuation knob 106.

Turning now to FIG. 11, the ring-shaped rotary selection knob 108 is shown mounted on the actuation shaft 102 with the plunger guide 138 (not shown) passing through a lumen 156 of the rotary selection knob 108. The plunger pin 110 is shown mounted in the plunger pin receiver 109 of the rotary selection knob 108 with a portion of the plunger pin 110 extending into the lumen 156 of the rotary selection knob 108. Thus, when the insertion instrument 90 is assembled, the plunger pin 110 engages the plunger guide 138 of the actuation shaft 102 and is moved laterally by rotation of the rotary selection knob 108 with respect to the actuation shaft 102. The longitudinally extending portions of the plunger guide 138 permit axial translation of the actuation shaft 102 with respect to the plunger pin 110 and rotary selection knob 108. Also, the rotary selection knob 108 can include labels or markings positioned to indicate the selected operation selected by the rotary selection knob 108 (i.e., Locked, Bladder, Urethra, Anastomosis, and Release).

Additionally, as shown in FIG. 11, the stopper cross-pin 104 is mounted within the stopper cross-pin opening 130 at the proximal end 128 of the actuation shaft 102. The stopper cross-pin 104 is adapted to restrict axial translation of the proximal end 128 of the actuation shaft 102 with respect to the hollow grip member 103 in a distal direction past the rotary actuation knob 106.

Referring now to FIGS. 12A and 12B, an example of a partially assembled handle assembly 92 is shown. In FIG. 12A, the rotary selection knob 108 and rotary actuation knob 106 are shown both mounted on the actuation shaft 102, with the rotary selection knob 108 being mounted proximally of the rotary actuation knob 106. As shown here, in the initial or “Locked” position, the adaptor 112 extends distally from the actuation shaft 102 and abuts the flexible body 94 which is fixed to the hollow grip member 103. The actuation shaft 102 with knobs 106, 108 are disposed within the hollow grip member 103.

In FIG. 12B, the handle assembly 92 is shown with only the deployer shaft 114 and adapter 112 mounted within the hollow grip member 103. As shown, the deployer shaft 114 extends through the hollow grip member 103 while the deployer shaft 114 passes through the lumen 151 of the adaptor 112, and would likewise pass through the lumen 122 of the actuation shaft 102 if the actuation shaft 102 were shown positioned in the hollow grip member 103.

Turning now to FIGS. 13A to 13B, detail of the implant mounting portion 99 of the implant support 98 is shown. The implant mounting portion 99 is generally cylindrical and comprises a first ring mounting portion 160 and a second ring mounting portion 162.

The first ring mounting portion 160 includes at least one axially extending first collar support member 164 and at least one axially extending and resiliently flexible first collar locking member 166. As seen best in FIG. 13B, the first collar 4 of the first ring assembly 2 is mountable on the first collar support member 164, with the first collar locking member 166 engaging the support surface 50 of the first collar 4. Thus, when the first collar locking member 166 axially extends and engages the support surface 50 of the first collar 4, as shown, the first collar locking member 166 restricts movement of the first collar 4 with respect to the implant support 98. However, a radially inward force applied to the first collar locking members 166 can cause the first collar locking members 166 to become disengaged from the first collar 4. When the first collar locking members 166 are disengaged from the first collar 4, the implant support 98 can slide through lumens 60 and 80 of the second central ring 56 and second collar 54 (see FIG. 6), such as during withdrawal of the insertion instrument 90.

As shown in FIG. 13C, the first central ring 6 is releasably retained on the deployer 100 of the insertion instrument 90 by protrusion of the deployer detent 113 into the circumferentially extending deployment slot 18 of the first central ring 6. As shown, the first central ring 6 is positioned distally with respect to the first collar 4, and the first ring securement elements 20 extend axially to a position within the outer circumference of the first collar 4.

Referring now to FIGS. 13A-13C, the second ring mounting portion 162 includes at least one flexibly resilient axially extending second ring support member 168 having proximally positioned a second ring undeployer cam 170 and a second ring deployer cam 171 positioned distally thereto. As best seen in FIG. 13B, the second ring undeployer cam 170 and the second ring deployer cam 171 are configured so that the second ring assembly wall 58 between the second ring securement element mounting members 64 of the second central ring 56 can be mounted on the second ring support members 168 between the second ring undeployer cam 170 and a second ring deployer cam 171. Thus, when the second ring support members 168 axially extend and the second central ring 56 is mounted thereon, the second ring undeployer cam 170 and a second ring deployer cam 171 restrict translation of the second central ring 56 with respect to the implant support 98. However, an inward force applied to the second ring support member 168 can cause the second ring support member 168 to become disengaged from the second central ring 56, thus allowing the implant support 98 to slide through lumens 60 and 80 of the second central ring 56 and second collar 54.

The second ring mounting portion 162 also includes at least one second ring securement element engaging cam member 163 extending axially from the implant mounting portion 99 of the implant support 98. The second ring securement element engaging cam members 163 are positioned between the second ring support members 168, about the circumference of the implant mounting portion. The second central ring 56 may be mounted on the second ring mounting portion 162 such that the second ring securement element engaging cam members 163 are positioned distally of and directed towards the second ring securement element cam surfaces 72.

Referring now to FIGS. 14A-14E, the movement of the actuation shaft 102 relative to the hollow grip member 103, during operation of the insertion instrument 90, is illustrated. As shown in FIG. 14A, in the initial or “Locked” position, the plunger pin 110 is received in the proximal portion of the plunger pin guide 138. To allow deployment of the first ring assembly 2, the rotary selection knob 108 (as seen in FIG. 12A) is rotated counter-clockwise (shown by the arrow x in FIG. 14A) to slide the plunger pin 110 through the plunger guide 138. From the “Locked” deployment position, counter clockwise rotation of the rotary selection knob 108 causes the plunger pin 110 to move within the plunger pin guide 138 to angle A1, thereby selecting the “Bladder” deployment position.

As shown in FIG. 14B, when the rotary selection knob 108 is in the “Bladder” deployment position, the insertion instrument 90 can deploy and undeploy the first ring assembly 2 to cause the first ring securement elements 20 to engage the surrounding tissue (i.e., bladder neck or other hollow body part). In the “Bladder” deployment position, the first ring assembly 2 can be deployed by proximal retraction of the actuator shaft 102 with respect to the hollow grip member 103 (not shown) and adapter 112, as shown by the arrow in FIG. 14B. Proximal retraction of the actuator shaft 102 can be effected by rotating the rotary actuation knob 106 (not shown) counter clockwise with respect to the actuator shaft 102, such that the threaded lumen 154 of the rotary actuation knob 106 engages the threaded portion 126 of the actuation shaft 102. As shown, engagement of the threaded portion 126 of the actuation shaft 102 during rotation of the rotary actuation knob 106 causes the actuation shaft 102 to move proximally such that the plunger guide 138 moves proximally about the plunger pin 110 and the position of the plunger pin 110 changes from A1 to A2. Because the deployer shaft 114 is fixed in lumen 122 of the actuation shaft 102, proximal translation of the actuation shaft 102 with respect to the hollow grip member 103 causes the deployer 100 to proximally retract with respect to the first collar 4, thereby deploying the first ring assembly 2 to engage the bladder or other tissue (discussed in detail below with respect to FIGS. 27A-27D).

Furthermore, as illustrated in FIG. 14B, proximal retraction of the actuation shaft 102 with respect to the adaptor 112 results in the adaptor guide receiver 148 to translate proximally about the adaptor guide 152 and causes the adaptor guide 152 to be engaged by the adaptor detent 150. Thus, with the actuator shaft 102 engaging the adaptor 112, further proximal translation of the actuation shaft 102 will carry the adaptor 112 in a coordinating motion.

Referring now to FIG. 14C, to select the insertion instrument 90 for partial deployment of the second ring assembly 52, the rotary selection knob 108 may be turned counterclockwise to carry the plunger pin 110 to position A3 of the plunger guide 138. When the plunger pin 110 is in position A3 of the plunger guide 138, the insertion instrument is in the “Urethra” deployment position. As shown in FIG. 14C, the rotary actuation knob 106 (not shown) can then be rotated counter clockwise with respect to the actuator shaft 102 to cause proximal retraction of the actuation shaft 102 with respect to the hollow grip member 103 such that the plunger pin guide 138 moves about the plunger pin 110 and the plunger pin 110 position changes from position A3 to A4.

Because the implant support shaft 101 is mounted on the adaptor 112, which is engaged by the actuation shaft 102 in the Urethra position, proximal retraction of the actuation shaft 102 results in proximal translation of the implant support 98 with respect to the hollow grip member 103 and outer housing 96. This proximal translation of the implant support 98, with respect to the hollow grip member 103 and outer housing 96, results in partial deployment of the second ring assembly 52 (discussed in detail below with respect to FIGS. 28A-28D).

As shown in FIG. 14D, to select the insertion instrument 90 for full deployment of the second ring assembly 52, the rotary selection knob 108 (not shown) may again be turned counterclockwise with respect to the actuation shaft 102, thereby carrying the plunger pin 110 to position A5. When the plunger pin 110 is in position A5 of the plunger guide 138, the insertion instrument 90 is in the “Anastomosis” position. The rotary actuation knob 106 (not shown) can then be rotated counter clockwise with respect to the actuator shaft 102 to again cause proximal retraction of the actuation shaft 102 with respect to the hollow grip member 103 (not shown). Retraction of the actuation shaft 102 with respect to the hollow grip member 103 when the plunger pin 110 is in position A5 shifts the position of the plunger pin 110 from A5 to A6 within the plunger guide 138. When the plunger pin 110 moves from A5 to A6 by proximal retraction of the actuation shaft 102 with respect to the hollow grip member 103, the result is further proximal translation of the implant support 98 with respect to the handle assembly 92 and outer housing 96. This further proximal translation of the implant support 98 results in full deployment of the second ring assembly 52 (discussed in detail below with respect to FIGS. 29A-29D).

As shown in FIG. 14E, approximation of the anastomosis can be achieved by further counter clockwise rotation of the rotary actuation knob 106 with respect to the actuator shaft 102 when the insertion instrument 90 is in the “Anastomosis” position. When the plunger pin 110 rests in position A6, rotation of the rotary actuation knob 106 with respect to the actuator shaft 102 causes the actuation shaft 102 to translate proximally with respect the handle assembly, thereby causing the plunger pin guide 138 to move around the plunger pin 110 until the plunger pin 110 is in position A7. Proximal translation of the actuation shaft 102 with respect to the hollow grip member 103 draws the first ring assembly 2 towards the second ring assembly 52 (discussed in detail below with respect to FIGS. 30A-30D). Furthermore, when the first ring assembly 2 and second ring assembly 52 are deployed and secured to the surrounding tissue (e.g., bladder and urethra, respectively), approximation of the first ring assembly 2 towards the second ring assembly 52 draws the hollow body parts, such as bladder and urethra tissue, towards anastomosis. Interconnecting engagement of the first ring assembly 2 and second ring assembly 52 secures the anastomosis.

Turning now to FIGS. 15A and 15B, a cross-section of the handle assembly 92 is shown to illustrate structures cooperating during the release of the first ring assembly 2 and second ring assembly 52 from the insertion instrument 90. As shown, the hollow grip member 103 includes hollow grip release detent 133, which extends into lumen 105 of the handle assembly 92. When the insertion instrument 90 is assembled, the hollow grip release detent 133 is disposed within the device guide slot 132 (not shown) and circumferentially extending recess 134 (as best seen in FIG. 9B). FIG. 15A shows the relative position of the hollow grip release detent 133 within the circumferentially extending recess 134 during insertion of the insertion instrument 90 and deployment and coupling of the second and first ring assemblies 52, 2 (i.e., the initial position, “Bladder” position, “Urethra” position, and “Anastomosis” position). FIG. 15B shows the relative position of the hollow grip release detent 133 within the circumferentially extending recess 134 during release of the second and first ring assemblies 52, 2 from the insertion instrument 90 (i.e. the “Release” position) and withdrawal of the insertion instrument 90 from the body.

As can be seen from FIGS. 15A and 15B, the second and first ring assemblies 52, 2 (not shown) can be released from the insertion instrument 90 subsequent to coupling to the second and first ring assemblies 52, 2 by rotation of the rotary selection knob 108 to the “Release” position past the actuation shaft detent 136. The engagement of the hollow grip release detent 133 with the actuation shaft detent 136 provides an audible and physically perceptible indication that the insertion instrument 90 (not shown) in the “Release” position. Furthermore, because the deployer shaft 114 (not shown) is fixed to the actuation shaft 102 (not shown), rotation of actuation shaft 102 results in coordinating motion of the deployer 100 (not shown). Rotation of the deployer 100 causes the deployer detent 113 and the deployer 100 to rotate within circumferentially extending deployment slot 18 of the first central ring 6 and into device release groove 16 (shown in FIG. 2). When the deployer detent 113 the deployer 100 is positioned in the device release groove 16 (not shown) of the first central ring 6 (not shown), the deployer 100 can slide through the lumen 10 of the first central ring 6. Furthermore, in the Release position, the deployer 100 and implant mounting portion 99 of the implant support 98 (not shown) can slide through the lumens 35, 60, 80 (not shown) of the first collar 4, second central ring 56 and second collar 54 (not shown).

Turning now to FIGS. 16-20, the insertion instrument 90 has flexible portions that allow manipulation of the insertion instrument 90, to adjust to the natural curvature of a patient's anatomical structures. As shown in FIG. 16, the insertion instrument 90 includes an optional shaft flexing portion 172 (also seen in FIGS. 17A and 18A). The shaft flexing portion 172 is defines a plurality of slits 174 defining a plurality of circumferential wall supports 176. As shown in FIG. 18A, the slits 174 define open areas within the implant mounting portion 99 of the implant support that, due to the absence of material, allow the wall supports 176 on the inner side 178 to converge and on the outer side 180 to spread further apart, thereby bending the shaft flexing portion 172.

Turning now to FIGS. 17A and 17B, the flexing assembly 182 on the insertion instrument 90, which provides for flexing of the shaft flexing portion 172 is shown. The flexing assembly 182 includes a control cable 184, which is mounted distally of a tension shaft 186 within the insertion instrument 90. The control cable 184 is an elastic flexible cable having a first end 188 and a second end 190. The tension shaft 186 is a resilient elongated member sized to slide through the lumen 118 of the implant support shaft 101 while the deployer shaft 114 is also passing through the implant support shaft 101. The tension shaft 186 has a length such that a portion extends proximally from the actuation shaft 102 and a portion extends into the implant mounting portion 99 of the implant support 98.

As seen best in FIG. 17B, the tension shaft 186 extends proximally through the actuation shaft 102 and is fixed to a trigger engaging member 192. The trigger engaging member 192 is sized so that it cannot pass through the actuation shaft and includes a trigger engaging lip 194. The trigger lip 194 is adapted to engage the trigger extension 196 of trigger 198, such that axial proximal translation of the trigger 198 with respect to the hollow grip member 103 carries the trigger engaging member 192 and the tension shaft 186 axially in a coordinating proximal movement.

The trigger 198 includes a finger pull 200 extending radially outward from the hollow grip member 103. The trigger engaging member 192 member can be proximally translated by pulling the finger pull 200 of the trigger 198 proximally with respect to the hollow grip member 103.

As best seen in FIGS. 17A and 17B, the first end 188 of the control cable 184 is fixed to deployer shaft 114, distally of the shaft flexing portion 172 of the implant support 98. The second end 190 of the control cable 184 is fixed to the tension shaft 186, proximally of the shaft flexing portion 172.

Turning now to FIGS. 18A and 18B, proximal pressure applied to the finger pull 200 carries the trigger 198 proximally, with respect to the hollow grip member 103. The proximal translation of the trigger 198 carries the trigger extension 196 proximally into engagement with the trigger lip 194, thus urging the trigger engaging member 192, and tension shaft 186 proximally with respect to the actuation shaft 102. Proximal translation of tension shaft 186 through lumen 122 of the actuation shaft 102 results in tension being applied to the control cable 184. Applied tension causes the deployer shaft 114 attached to the first end 188 of the control cable 184 to flex. As shown in FIG. 18A, the flexing of the deployer shaft 114 causes the shaft flexing portion 172 of the implant support 98 to flex as well. Tension due to the elasticity of the flexing assembly 182 urges the flexing portion 172 to straighten upon release of pressure on the finger pull 200. As will be readily apparent to those skilled in the art, other means may also be used to effectuate directional movement of the deployer 100.

Turning now to FIG. 19, the insertion instrument 90 also has passive flexibility to allow further conformance to anatomical features. The flexible tube 94, implant support shaft 101, and deployer shaft 114 (located internally of flexible tube 94) are formed of resilient flexible material such that the insertion instrument can flex and bend to yield to resistance encountered during insertion of the insertion instrument 90 into curved anatomical structures.

As shown in FIGS. 20 and 21, in one embodiment, the insertion instrument 90 may include an optional shaft flexing portion 202 positioned proximally of the outer housing 96. The positioning of the flexing portion 202 permits 360° motion of the insertion instrument 90 extending distally from the junction between the flexible tube 94 and outer housing 96 and may be operated similarly to the flexing assembly 182 discussed in FIGS. 16 to 18. The shaft flexing portion is formed of circumferential grooves 204, which decrease the thickness of the outer housing 96, thereby concentrating flexibility in a similar manner to the flexing assembly 182.

Turning now to FIG. 22, an alternate embodiment of a handle assembly 1092 is shown. The alternate handle assembly 1092 is provided with a device release switch 1007. Thus, rather than releasing the ring assembly 1003 from the insertion instrument 1092 by operation of the rotary selection knob 1008, as discussed with the knob 108, the ring assembly 1003 is released by depression of the device release switch 1007.

In FIGS. 22A and 22B and FIGS. 24A and 24B, alternate shapes of the hollow grip member 1103/1203 are shown. As shown in FIGS. 23A and 23B, the hollow grip member 1103 is a straight symmetrical shape. In contrast, as shown in FIGS. 24A and 24B, the hollow grip member 1103 is spherical. Alternate shapes suitable for comfortably gripping the hollow grip member 1103/1203 of the handle assembly 1192/1292 are also contemplated.

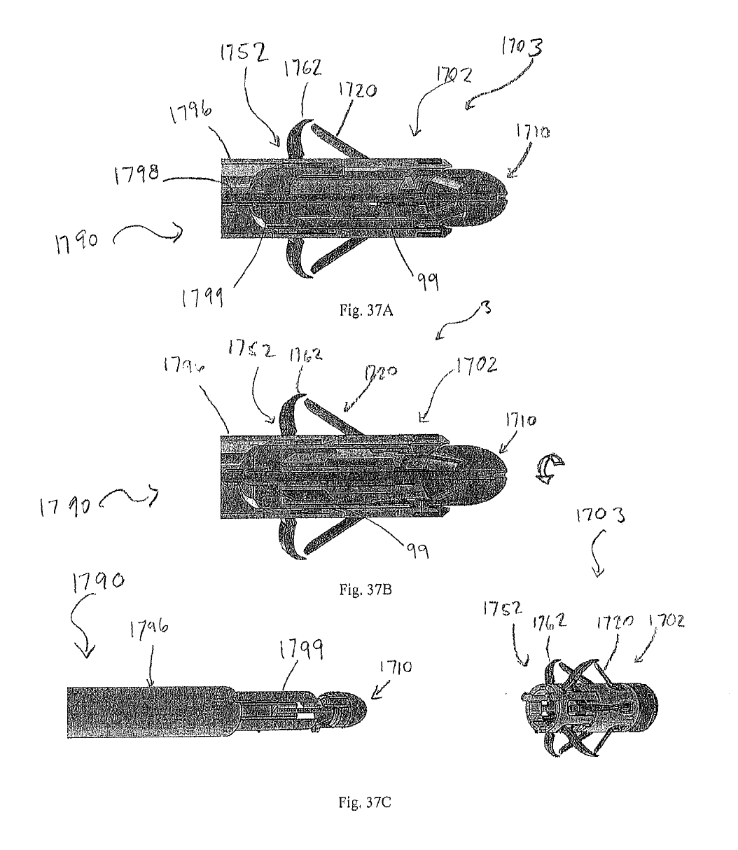

One skilled in the art will appreciate that alternate embodiments may incorporate different structures or designs for release of the ring assembly. One example of an alternate embodiment of a design for releasing the ring assembly 1703 from the insertion instrument 1790 is shown in FIGS. 37A-37C. As shown in FIG. 37A, the second ring assembly 1752 is mounted on the implant mounting portion 1799 of the implant support 1798 and the first ring assembly 1702 is mounted on the deployer 1710 during anastomosis. As shown in FIG. 37B, when the insertion instrument 1790 is operated to release the ring assembly 1703, the implant mounting portion 1799 of the implant support 1798 and the deployer 1710 are simultaneously rotated counter-clockwise with respect to the outer housing 1796 and the ring assembly 1703, as shown by the arrow in FIG. 37B. Rotation of the implant mounting portion 1799 and deployer 1710 with respect to the ring assembly 1703 disengages the ring assembly 1703 from the insertion instrument 1790, thereby allowing proximal translation of the insertion instrument 1790 away from the ring assembly 1703. As shown in FIG. 37C, translation or movement of the insertion instrument 1790 away from the ring assembly 1703 subsequent to disengagement of the ring assembly 1703 results in withdrawal of the insertion instrument 1790 from the patient and leaves the ring assembly 1703 in place holding anastomosis.

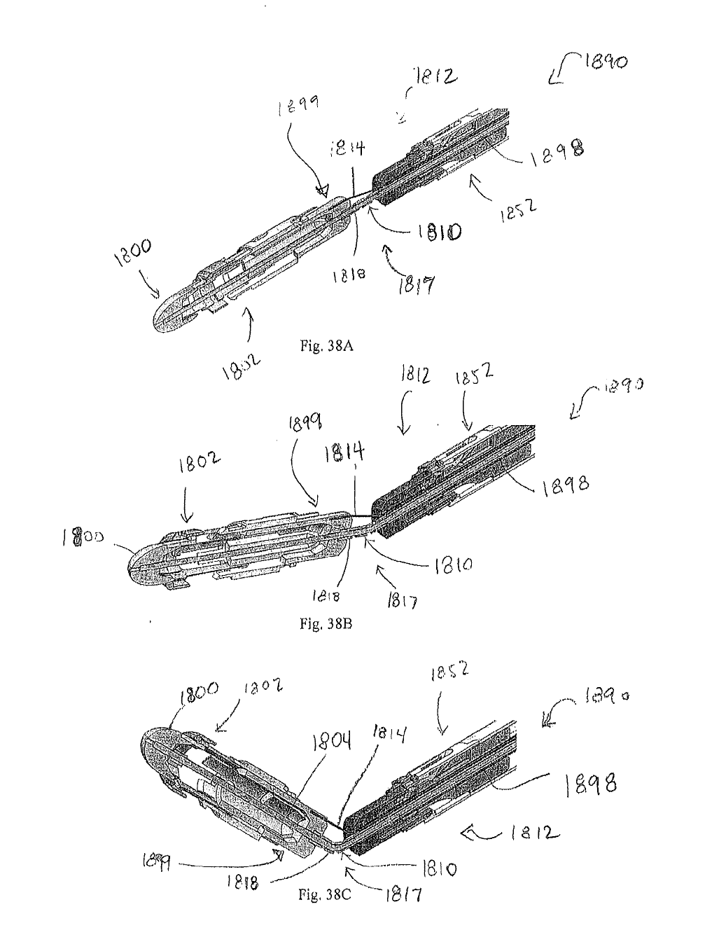

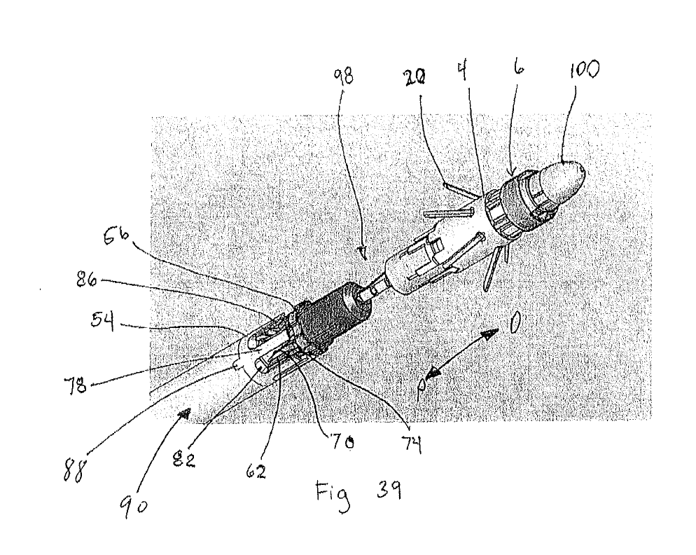

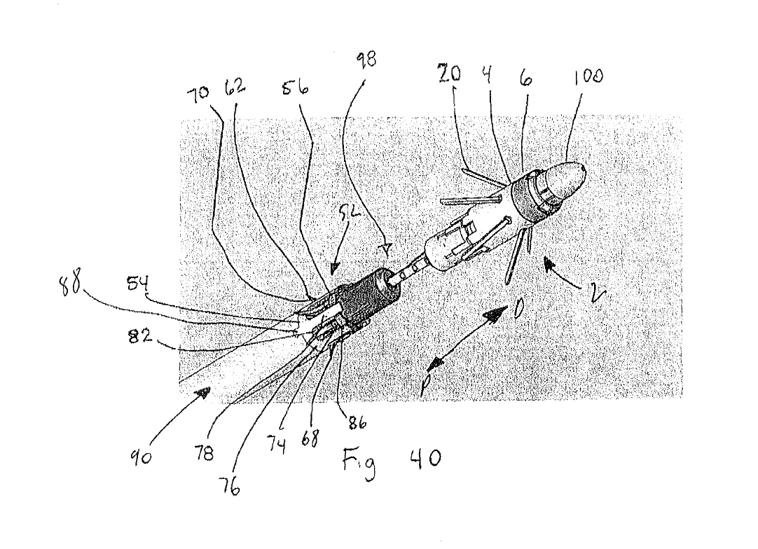

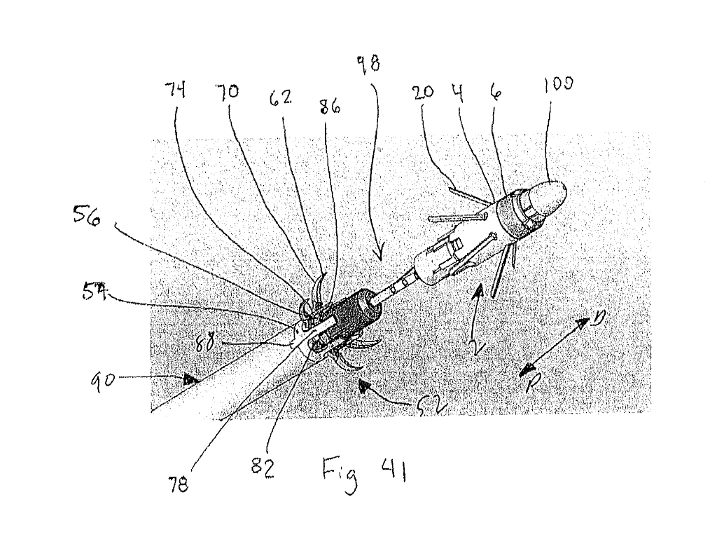

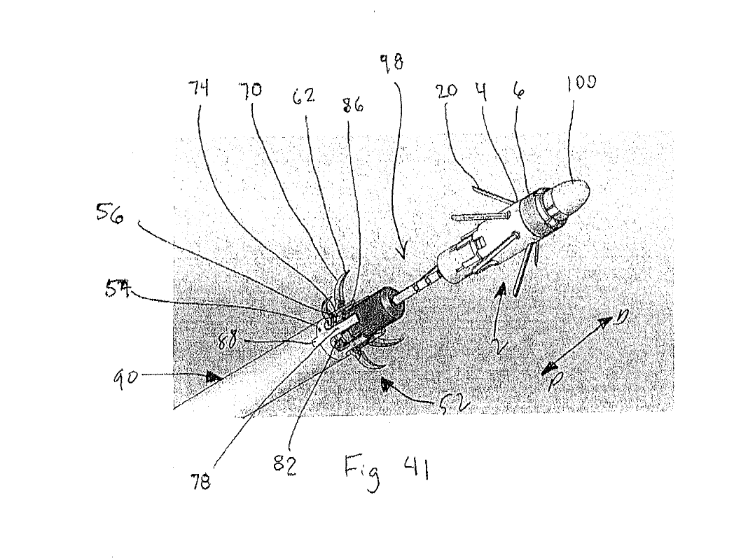

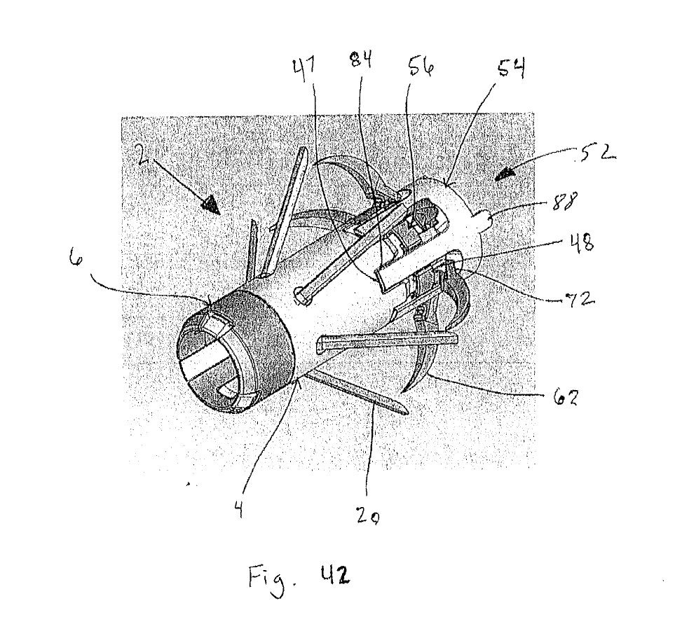

Additionally, one skilled in the art will appreciate that alternate designs for achieving flexibility or manipulability of an insertion instrument are possible, such as the embodiment of an insertion instrument 1890 depicted in FIGS. 38A to 38C. As shown in FIG. 38A, the insertion instrument 1890 includes a shaft flexing portion 1817 defined by the implant support shaft 1810. The shaft flexing portion 1817 defines a flexible tube having a plurality of segments 1818. As shown in FIG. 38A, the segments 1818 define open areas within the implant support shaft 1810 that, due to the absence of material, allow convergence towards or divergence from adjacent segments 1818, thereby allowing bending of the shaft flexing portion 1817.

Still referring to FIG. 38A, the flexing assembly 1812 of the insertion instrument 1890 includes a control wire 1814, which is mounted to the implant mounting portion 1899 of the implant support 1898 and to a trigger mechanism (not shown) on the handle portion of the insertion instrument 1890. The trigger mechanism (not shown) can be operated to apply tension to the control wire 1814, thereby causing the shaft flexing portion 1817 of the implant support shaft 1810 to bend or flex.

Implantation Method

Referring to FIGS. 25-32, an exemplary method of using an insertion instrument 90 to create anastomosis of two vessels is shown. Although many types of anastomoses are possible using the device disclosed herein, an exemplary anastomosis of a bladder and urethra, such as one that may occur following removal of the prostate, is shown. While these figures depict the anastomosis of a bladder and urethra, the same or similar techniques should be understood as applying to the anastomosis of any other hollow organs or vesicles, such as blood vessels or intestines. Access to the anastomosis site may be achieved using natural orifices, such as the urethra as shown in FIGS. 25-32, suprapubicly, through incision or any other access port or via surgical means. As will be recognized by those skilled in the art, the specific insertion means will be determined by the type of anastomosis being performed and the available access areas in the specific body location where such anastomosis is being performed.

As depicted in FIG. 25, the anastomosis system 1 is inserted through the urethra to position first ring assembly 2 within a first hollow body part, such as a bladder neck, by pushing hollow grip member 103 of handle assembly 92 (not shown) to advance the insertion instrument 90 through the second hollow body part, such as a urethra. FIGS. 26A-26D show the arrangement of the insertion instrument 90 during insertion. As shown, the second ring assembly 52 and first ring assembly 2 are mounted on the second ring assembly mounting portion 162 and first ring mounting portions 160, respectively. Both the first and second ring assemblies 2, 52 are in the undeployed or retracted position. The insertion instrument 90 is in the “Locked” position.

Turning now to FIGS. 27A-27D, the deployment of the first ring securement elements 20 of the first ring assembly 2 is shown. As shown in FIG. 27B, once the first ring assembly 2 is aligned at a suitable position within the first hollow body part (e.g. bladder neck), the rotary selection knob 108 is rotated counter-clockwise (in the depicted embodiment, the angle of rotation is 72°; however, other degrees of rotation are contemplated). As discussed above with respect to FIG. 14B, rotation of the rotary selection knob 108 from the initial “Locked” position selects a deployment position, such as the “Bladder” deployment position shown here. When the rotary selection knob 108 is in the “Bladder” deployment position, counter-clockwise rotation of the rotary actuation knob 106 with respect to the handle assembly 92 results in axial translation of the actuation shaft 102 in the direction of the handle assembly, i.e. in the proximal direction. As shown in FIG. 27C, proximal translation of the actuation shaft 102 also carries the deployer shaft 114 in the proximal direction with respect to the handle assembly 92 and through the lumens 117 and 118 of the flexible tube 94 and implant support 98 (indicated by arrows “x” in FIGS. 27A, 27C and 27D).

As shown in FIG. 27D, because the deployer 100 is mounted on the deployer shaft 114 and the first central ring 6 is mounted to the deployer 100, translation of the deployer shaft 114 towards the handle 92 carries the first central ring 6 axially towards the first collar 4. As discussed with respect to FIGS. 4 and 5 and shown in FIG. 27B, as the first central ring 6 advances into the first collar 4, the guide surfaces 34 of the first collar 4 displace the first ring securement elements 20, which are urged to bend and deploy radially outward and proximally from the first collar 4 (shown by arrow “y” in FIGS. 27A, 27C and 27D).

As shown in FIG. 27B, the deployment of the first ring securement elements 20 when the first ring assembly 2 is in position in the first hollow body part (e.g., bladder neck) causes the first ring securement elements 20 to pierce and engage the hollow body part tissue. As shown, the first ring securement elements 20 secure the first hollow body part (e.g., bladder neck) by being driven into the tissue in a generally proximal and radially outward direction. Although not shown, a surgeon may also compress the first hollow body part (e.g., bladder neck) tissue around the first ring assembly 2 to ensure that the first ring securement elements 20 securely engage the first hollow body part. Additionally or alternatively, the surgeon may gently pull the insertion instrument 90 in the proximal direction with respect to the first hollow body part (e.g., bladder) to secure and/or maintain engagement of the first ring securement elements 20 with the first hollow body part.

As shown in FIG. 27A, the first ring assembly 2 can be undeployed by clockwise rotation of the rotary actuation knob 106 with respect to the hollow grip member 103 to cause the deployer shaft 114 and deployer 100 to axially extend in the distal direction with respect to the first collar 4, thereby carrying the first central ring 6 away from the first collar 4. As a result, if proper attachment to the first hollow body part (e.g., bladder) is not achieved initially, the first ring securement elements 20 may be retracted and redeployed.

Turning now to FIGS. 28A-28D, partial deployment of the second ring assembly 52 to engage the second hollow body part (e.g. urethra) is shown. As shown in FIGS. 28A and B, once the first ring assembly 2 is secured in the first hollow body part (e.g., bladder), the second ring assembly 52 is aligned at a suitable position within the second hollow body part (e.g., urethra neck), the rotary selection knob 108 is rotated counter-clockwise (in the depicted embodiment, the angle of rotation is 72°; however, other degrees of rotation are contemplated). As discussed above with respect to FIG. 14C, rotation of the rotary selection knob 108 from the “Bladder” deployment position selects the “Urethra” deployment position.

When the rotary selection knob 108 is in the “Urethra” deployment position, counter-clockwise rotation of the rotary actuation knob 106 with respect to the handle assembly 92 results in axial translation of the actuation shaft 102 in the proximal direction with respect to the hollow grip member 103. Furthermore, because the adapter 112 is engaged by the actuation shaft 102 (as seen in FIG. 14C) when the rotary selection knob 108 is in the “Urethra” deployment position, proximal translation of the actuation shaft 102 carries the adapter 112 and the implant support 98 mounted thereto in a coordinating proximal movement. Thus, as shown in FIG. 28C, proximal translation of the actuation shaft 102 and adapter 112 when the rotary selection knob 108 is in the “Urethra” deployment position carries the implant support 98 and deployer shaft 114 in the proximal direction through the lumens 117 and 119 of the flexible tube 94 and urethra side cam 116 and the implant mounting portion 99 of the implant support 98 into the outer housing 96 (indicated by arrows “x” in FIGS. 28A, 28B, 28C and 28D).

As shown in FIG. 28D, the second collar 54 is mounted on the second collar mounting portion 97 of the outer housing 96, such that proximal translation of the implant mounting portion 99 of the implant support 98 through the outer housing 96 carries the second central ring 56 into sliding engagement with the second collar 54. Thus, the proximal translation of the implant support 98 drives the second ring securement elements 62 into contact with the angled second ring securement element engagement surface 82 of the second collar 54 and the urethra side cam 116. As discussed with respect to FIG. 6 and shown in FIG. 28B, engagement of the second ring securement elements 62 with the angled second ring securement element engagement surfaces 82 of the second collar 54 and the urethra side cam 116 displaces the second ring securement elements 62, thereby urging the second ring securement elements 62 radially outward (shown by arrow “y” in FIGS. 28A, 28B and 28D). The second central ring 56 slides into the second collar 54 until the second ring securement element mounting member 64 of the second central ring 56 contacts the proximal ring base 76 of the second collar 54.

As shown in FIG. 28B, the radial deployment of the second ring securement elements 62 when the second ring assembly 52 is in position in the second hollow body part causes the second ring securement elements 62 to pierce and engage the second hollow body part, such as a urethra neck. As shown, the second ring securement elements 62 secure the second hollow body part by being driven into the tissue in a generally radially outward direction.

Additionally, as shown in FIG. 28A, the second ring assembly 52 can also be undeployed by clockwise rotation of the rotary actuation knob 106 with respect to the hollow grip member 103 to cause the implant support 98 and deployer 100 to axially extend in the distal direction with respect to the second collar 54, thereby carrying the second central ring 56 away from the second collar 54.

Turning now to FIGS. 29A-29D, full deployment and securement of the second ring securement elements 62 in the second hollow body part (i.e., urethra) is shown. As shown in FIG. 29B, once the first ring assembly 2 is secured in the first hollow body part (e.g., bladder) and the second ring assembly 52 is partially deployed within the second hollow body part (i.e., urethra neck), the rotary selection knob 108 is rotated counter-clockwise (in the depicted embodiment, the angle of rotation is 72°; however, other degrees of rotation are contemplated). As discussed above with respect to FIG. 14D, rotation of the rotary selection knob 108 from the “Urethra” deployment position selects the “Anastomosis” deployment position.

When the rotary selection knob 108 is in the “Anastomosis” deployment position, counter-clockwise rotation of the rotary actuation knob 106 with respect to the hollow grip member 103 results in axial translation of the actuation shaft 102 in the proximal direction with respect to the hollow grip member 103. As shown in FIG. 29C, proximal translation of the actuation shaft 102 when the rotary selection knob 108 is in the “Anastomosis” deployment position carries the implant support shaft 101 and deployer shaft 114 further in the proximal direction through the lumens 117 and 119 of the flexible tube 94 and urethra side cam 116 and the implant mounting portion 99 of the implant support 98 further into the outer housing 96 (indicated by arrows “x” in FIGS. 29A, 29B and 29D).

As shown in FIG. 29B, in the “Anastomosis” deployment position, the second ring securement element mounting member 64 engages the second collar 54, thereby preventing further sliding of the second central ring 56 into the second collar 54. Furthermore, because the second collar 54 is mounted on the outer housing 96 as shown, the outer housing 96 causes the second collar 54 and second central ring 56 to resist further axial movement. Thus, with the second ring assembly 52 resisting further axial translation with respect to the outer housing 96, the force applied by proximal translation of implant support 98 with respect to the outer housing 96 drives the second ring support members 168 (see FIG. 13A) inward, thereby disengaging the second central ring 56 from the implant support 98.

With the second central ring 56 disengaged from the implant support 98, the implant mounting portion 98 can translate proximally with respect to the second central ring 56 when the implant support 98 is carried proximally by the actuation shaft 102. Furthermore, as the implant mounting portion 98 translates proximally with respect to the second central ring 56, the second ring securement element engaging cam members 163 of the implant mounting portion 99 of the implant support 98 are driven into contact with the second ring securement element cam surfaces 72 of the second ring securement elements 62, which are pivoted to extend into the lumen 60 of the second central ring 56. Engagement of the second ring securement element engaging cam members 163 with the second ring securement element cam surfaces 72 of the second ring securement elements 62 during proximal translation of the implant support 98 urges the second ring securement elements 62 to pivot further outward until the second ring securement element cam surfaces 72 are axially aligned with the second ring securement element mounting members 64. As shown in FIG. 29B, the second ring securement elements 62 are fully deployed and are generally directed distally to secure the second hollow body part, such as a urethra.

Turning now to FIGS. 30A-30D, approximation of the first ring assembly 2 and the second ring assembly 52 and anastomosis of the hollow body parts, such as a urethra and bladder, is shown. As shown in FIG. 30B, the first ring assembly 2 is secured in the bladder and the second ring assembly 52 is fully deployed and secured within the urethra neck. The rotary selection knob 108 is not rotated and the insertion instrument 90 remains in the “Anastomosis” deployment position. Counter-clockwise rotation of the rotary actuation knob 106 with respect to the hollow grip member 103 results in axial translation of the actuation shaft 102 in the proximal direction with respect to the hollow grip member 103. As shown in FIG. 30C, further proximal translation of the actuation shaft 102 when the rotary selection knob 108 is in the “Anastomosis” deployment position following full deployment of the second ring assembly 52 carries the implant support shaft 101 and deployer shaft 114 further in the proximal direction with respect to the flexible tube 94, urethra side cam 116 and outer housing 96 and through the lumens 117 and 119 of the flexible tube 94 and urethra side cam 116 (indicated by arrows “x” in FIGS. 29A to 29D).

Furthermore, as shown in FIG. 30D, the implant mounting portion 99 of the implant support 98 is also carried further into the outer housing 96, though lumens 60 and 80 of the second central ring 56 and second collar 54 and into engagement with the urethra side cam 116. Proximal movement of the implant mounting portion 99 of the implant support 98 through the outer housing 96 displaces the urethra side cam 116 and the urethra side cam 116 is pushed proximally with respect to the outer housing 96 by the implant support 98. Additionally, proximal translation of the implant mounting portion 99 of the implant support 98 carries the first ring assembly 2, and the first hollow body part tissue (i.e., bladder tissue) secured thereto towards contact with the second ring assembly 52, and the second hollow body part tissue (i.e., urethra tissue) secured thereto. As shown, the cut portion of bladder B1 at least partially engages the cut portion of the urethra U1 to form an end-to-end anastomosis, although end-to-end anastomosis of other hollow body parts may be achieved by the same or similar methods.

As shown in FIG. 30D, when the first ring assembly 2 is brought into engagement with the second ring assembly 52, the first ring assembly 2 and second ring assembly 52 couple together due to engagement of the first ring interconnecting elements 47 with the second ring interconnecting elements 84. Specifically, due to axial alignment of the first ring interconnecting elements 47 with the second ring interconnecting elements 84, translation of the first ring assembly 2 into contact with the second ring assembly 52 urges the first ring interconnecting elements 47 into connecting engagement with the second ring interconnecting elements 84 by a snap- or press-fit connection.

Furthermore, due to the axial alignment of the second central ring locks 86 with the support surfaces 50 of the first collar 4, translation of the first ring assembly 2 into engagement with the second ring assembly 52 urges the second central ring locks 86 against the support surfaces 50 of the first collar 4 and inwardly displaces the first collar locking member 166. Inward displacement of the first collar locking member 166 disengages the first collar locking member 166 from the first collar 4 and allows the implant mounting portion 99 of the implant support 98 to slide through lumens 60 and 80 of the second collar 54 and the second central ring 56.

Simultaneously, due to the axial alignment of the second ring securement element locking members 48 of the first collar 4 and the second ring securement element cam surfaces 72, translation of the first ring assembly 2 into engagement with the second ring assembly 52 urges the second ring securement element locking members 48 of the first collar 4 into engagement with the second ring securement element cam surfaces 72. Engagement of the second ring securement element locking members 48 of the first collar 4 with the second ring securement element cam surfaces 72 resists pivoting of the second ring securement element cam surfaces 72 into the lumen 60 of the second central ring 56 and supports the second ring securement elements 62 in the fully deployed position.

Turning now to FIGS. 31A-31D, release of the first ring assembly 2 and second ring assembly 52 from the insertion instrument 90 following coupling of the first and second ring assemblies 2, 52 to form an anastomosis is shown. As shown in FIG. 31B, once the first ring assembly 2 and second ring assembly 52 are secured to the tissue and coupled together, the rotary selection knob 108 is rotated counter-clockwise by 72°. Rotation of the rotary selection knob 108 from the “Anastomosis” deployment position selects the “Release” position.

Rotation of the rotary selection knob 108 to the “Release” position rotates the actuation shaft 102 counter-clockwise with respect to the hollow grip member 103. Rotation of the actuation shaft 102 causes circumferentially extending recess 134 of the actuation shaft 102 to slide against the hollow grip release detent 133 of the hollow grip member 103 and the actuation shaft detent 136 to engage the hollow grip release detent 133. Furthermore, because the deployer shaft 114 is mounted to the actuation shaft 102 and the deployer 100 is mounted to the deployer shaft 114, the deployer 100 also rotates counter-clockwise with respect to the hollow grip member 103 of the handle assembly 92.

As seen in FIG. 31A, rotation of the deployer 100 causes the deployer detent 113 of the deployer 100 to slide through circumferentially extending deployment slot 18 of the first central ring 6 and into device release groove 16. When the deployer detent 113 of the deployer 100 is positioned in the device release groove 16 of the first central ring 6, the deployer 100 can slide through the lumen 10 of the first central ring 6. Furthermore, in the “Release” position, the deployer 100 and implant mounting portion 99 of the implant support 98 can slide through the lumens 35, 60, 80 of the first collar 4, second central ring 56 and second collar 54.

Turning now to FIGS. 32A-32D, the withdrawal of the insertion instrument 90 from the body following release of the first and second ring assemblies 2, 52 is shown. As shown in FIG. 32B, the second and first ring assemblies 52, 2 are secured to the tissue. Accordingly, as shown in FIG. 32A, proximal translation of the handle assembly 92 through the second hollow body part withdraws the insertion instrument 90 from the patient. The instrument engaging element 88 releases the second collar 54 from the outer housing 96 of the insertion instrument 90 when the second ring assembly 52 is secured to the second hollow body part and the insertion instrument 90 is translated away from the second ring assembly 52 leaving the second and first ring assemblies 52, 2 coupled together to hold the hollow body parts, such as a urethra and bladder, in anastomosis. The second and first ring assemblies 52, 2 may be removed after a period of healing or, alternatively, may be permitted to biodegrade in place.

### Additional Embodiments

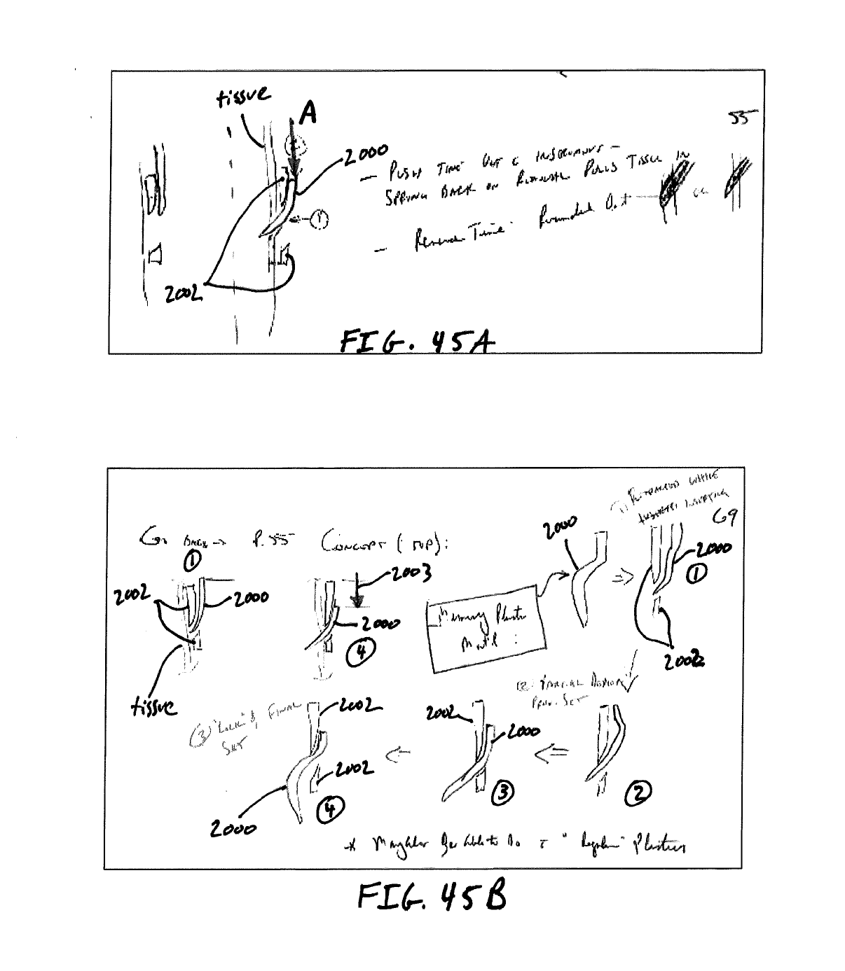

FIG. 45A depicts a further embodiment of an anastomosis device. This embodiment includes a tissue engaging structure 2000 that is operable in connection with an anastomosis structure 2002 to engage tissue within a patient's vessel or other body part. The tissue engaging structure 2000 may be pointed or curved at its tip, and may be biased to force tissue towards the anastomosis structure 2000, once inserted in a vessel. To deploy the tissue engaging structures 2000, the tissue engaging structures 2000 are moved relative to the anastomosis structure in the direction of arrow A.

FIG. 45B depicts the anastomosis device shown in FIG. 45A in various positions with respect to the tissue of a patient. The tissue engaging structure 2000 that is operable in connection with an anastomosis structure 2002 to engage tissue within a patient's vessel. The tissue engaging structure 2000 may be pointed or curved at its tip and may be biased to force tissue towards the anastomosis structure 2002, once inserted in a vessel. Also, the tissue engaging structure 2000 may be made from shape memory material such as plastic or nitinol, such that it resumes its original shape after being flexed due to pressure or tension. As shown in FIG. 45B, there are various stages of insertion into a patient's tissue, depicted by the numbers 1-4, with stage “1” being the initial step, and stage “4” showing the tissue engaging structure 2000 inserted into a patient's tissue. The embodiment of FIGS. 45A and 45B may be used with the insertion devices and procedures disclosed herein, or any other device or procedure that may facilitate deployment of the tissue engaging structures 2000.

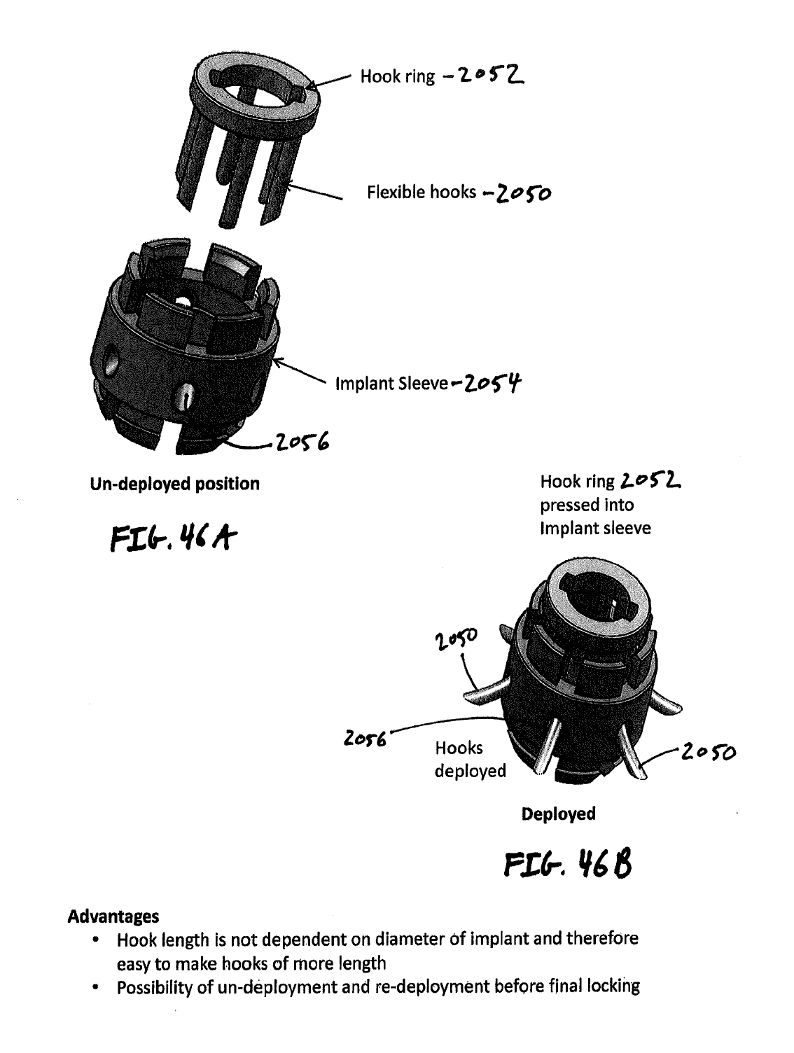

FIG. 46A depicts a further embodiment of an anastomosis device shown in an undeployed position. In the embodiment shown, there are a number of “flexible hooks” or tissue engaging structures 2050 extending axially from a “hook ring” 2052 and adapted to cooperate with an “implant sleeve” 2054 to deploy the flexible hooks 2050 outwardly when the hook ring 2052 is moved to engage the implant sleeve 2054. The embodiment of FIG. 46A is similar to that discussed herein with respect to FIGS. 4 and 5, and operable in a similar manner.

FIG. 46B depicts the anastomosis device of 46A in the deployed position. Specifically, the hook ring 2052 has engaged the implant sleeve 2054 by being pressed or moved axially into the implant sleeve 2052. The flexible hooks 2050 have deployed by being lined up with apertures 2056 in the implant sleeve 2054 and flexed outwardly as they pass through the apertures 2056. The embodiment of FIGS. 46A and 46B may be used with the insertion devices and procedures disclosed herein, or any other device or procedure that may facilitate deployment of the tissue engaging structures 2052.

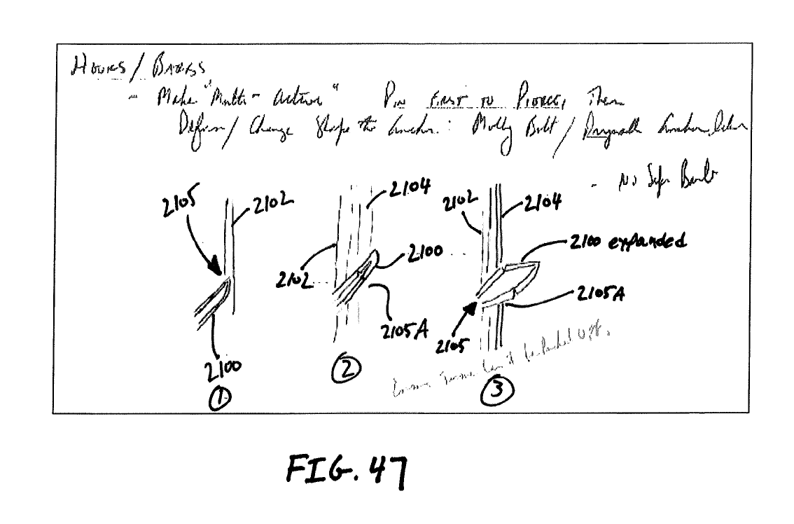

FIG. 47 depicts a further embodiment of an anastomosis device, shown in various stages of deployment. Specifically shown in FIG. 47 is a tissue engaging structure 2100 positioned with respect to an anastomosis sleeve 2102, proximate a patient's tissue 2104. The tissue engaging structure 2100 is made up of a material having flexible properties, such that after deployment it is biased to flex into an expansive shape. The first stage, depicted with a “1” is shown with the tissue engaging structure 2100 in an un-deployed position, which is virtually a straight line. The straight position of the tissue engaging structure 2100 may be retained by virtue of inward pressure on the tissue engaging structure by the walls of the anastomosis sleeve 2102. Stage “2” shows the tissue engaging structure 2100 partially extended from the anastomosis sleeve 2012, and partially penetrating the tissue 2104. As in stage “1” the tissue engaging structure 2100 is retained in a straight configuration. Finally, in stage “3,” the tissue engaging structure 2100 is fully extended through the tissue 2104. The tissue engaging structure 2100 has expanded to a width and shape that is larger than the insertion hole 2105 in the anastomosis sleeve and the puncture hole 2105A created in the tissue 2104, thus forming a mechanical engagement of the tissue 2104 between the tissue engaging structure and the anastomosis sleeve 2102 anchoring or securing the anastomosis sleeve 2102 in place. In the embodiment shown, the outward flexing of the tissue engaging structure 2100 is facilitated by hinge portions 2106, such as a living hinge; however, those skilled in the art will recognize that many other flexing features may be utilized to facilitate flexing of the tissue engaging structure 2100 to engage the tissue 2104. The embodiment of FIG. 47 may be used with the insertion devices and procedures disclosed herein, or any other device or procedure that may facilitate deployment and expansion of the tissue engaging structures 2100.

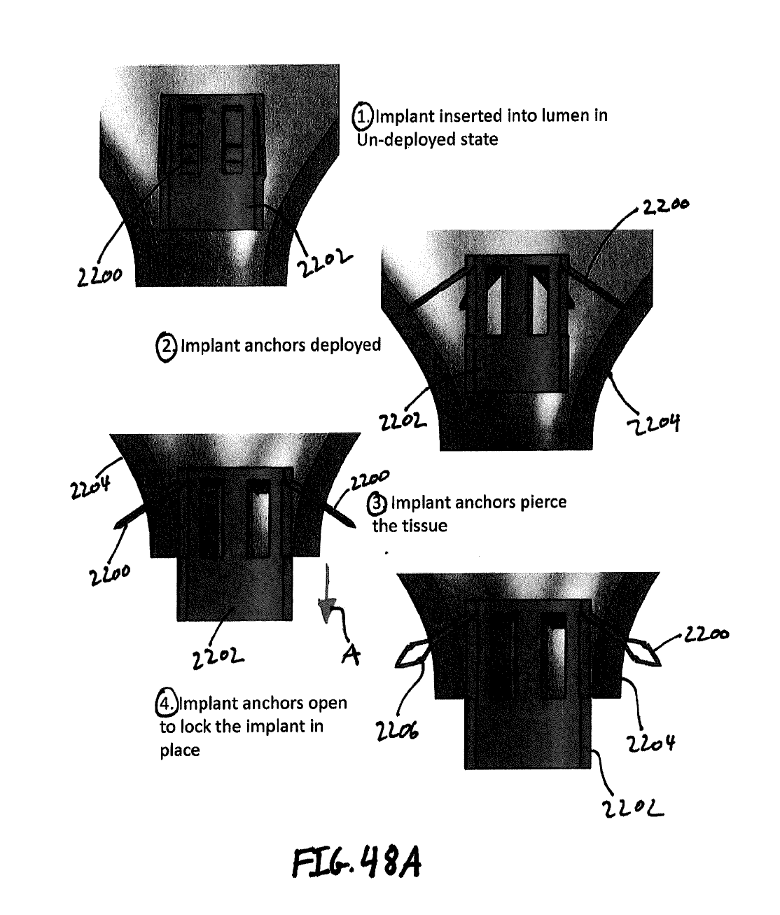

FIG. 48A depicts a further embodiment of an anastomosis device, shown in various stages (1-4) of deployment. The device of FIG. 48A includes similar structures to the device of FIG. 47, and operates in a similar manner. A tissue engaging structure 2200 is positioned with respect to an anastomosis sleeve 2202, proximate a patient's tissue 2204. The tissue engaging structure 2200 is made up of a material having flexible properties, such that after deployment, it is biased to flex into an expansive shape. The first stage, depicted with a “1” is shown with the tissue engaging structure 2200 in an un-deployed position, which is virtually a straight line, and held within the outer diameter of the anastomosis sleeve 2202. The straight position of the tissue engaging structure 2200 may be retained by virtue of inward pressure on the tissue engaging structure by the walls of the anastomosis sleeve 2202. Stage “2” shows the tissue engaging structure 2200 partially extended from the anastomosis sleeve 2202, and ready to penetrate the tissue 2204. As in stage “1” the tissue engaging structure 2200 is retained in a straight configuration. Stage “3” shows the tissue engaging structure 2200 penetrating and piercing the tissue 2204. In order to have the tissue engaging structure 2200 pierce the tissue 2204, once the tissue engaging structures 2200 are deployed, the anastomosis sleeve 2202 is, for example, moved in the direction of arrow A. As in stage “1” the tissue engaging structures 2200 are retained in a straight configuration. Finally, in stage “4,” the tissue engaging structures 2200 are fully extended through the tissue 2204 and expanded to a width and shape that is larger than the penetration hole created in the tissue 2204, thus forming a mechanical engagement of the tissue 2204 between the expanded tissue engaging structure 2200 and the anastomosis sleeve 2202, thereby anchoring or securing the anastomosis sleeve 2202 in place. In the embodiment shown, the outward flexing of the tissue engaging structure 2200 is facilitated by hinge portions 2206, such as a living hinge; however, those skilled in the art will recognize that many other flexing features may be utilized to facilitate flexing of the tissue engaging structure 2200 to engage the tissue 2204.

FIG. 48B depicts the anastomosis device of FIG. 48A in place in a patient's vessel, such as a bladder. The device is shown in the deployed position (Stage “4”) referred to above with respect to FIG. 48A with the tissue engaging structures 2200 fully extended through the tissue 2204, and expanded by flexing at the hinges 2206. The embodiment of FIGS. 48A and 48B may be used with the insertion devices and procedures disclosed herein, or any other device or procedure that may facilitate deployment and expansion of the tissue engaging structures 2200.

FIG. 49 depicts a further alternative embodiment of a tissue engaging structure 2300 for use with anastomosis devices disclosed herein. The tissue engaging structure 2300 includes a pointed distal tip 2302, a cylindrical shaft 2303, and a sheath 2304, which may be made from a flexible material. As can be seen in FIG. 49, the sheath 2304 attaches to an anastomosis ring 2306 at one end and attaches to a recessed portion 2305 of the cylindrical shaft 2303 at the other end by way of a ring portion 2307 of the sheath 2304 that is received in the recessed portion 2305. The distal tip 2302, cylindrical shaft 2303, and sheath 2304 extend outwardly from the anastomosis ring 2306 to engage a patient's tissue portion 2308. Stage “1” shows the tissue engaging structure 2300 partially inserted through a tissue portion 2308. Stage “2” shows the tissue engaging structure 2300 fully inserted through the tissue portion 2308 such that the sheath 2304 is fully stretched. When the sheath 2304 is fully stretched and a predetermined force on the ring portion 2307 of the sheath 2304 is reached, the ring portion 2307 of the sheath 2304 is pulled out of the recessed portion 2305. When this happens, as depicted in Stage “3,” the sheath collapses into its lowest energy state in bulk onto the tissue portion 2308 at the base of the cylindrical shaft 2303. This bulky mass of sheath material traps the tissue portion 2308 between itself and the anastomosis ring 2306, preventing the tissue engaging structure 2300 from pulling out of the tissue portion 2308. Also, because the diameter of the cylindrical shaft and bulky sheath material is greater than the hole in the tissue portion 2308 caused by penetration of the tissue engaging structure 2300, a mechanical fit is also formed between the tissue portion 2308, the tissue engaging structure 2300 and the anastomosis ring 2306. In other embodiments, the tissue engaging structure 2300 may be curved. The embodiment of FIG. 49 may be used with the devices and procedures set forth herein, or any other device or procedure that may facilitate insertion of the tissue engaging structure 2300 into a tissue portion.

FIG. 50 depicts a further alternative embodiment of tissue engaging structure shown in various stages (1-3) of deployment. The tissue engaging structure 2400 is similar to the device 2300 discussed with respect to FIG. 49 and includes a cylindrical shaft (not shown) having a pointed distal tip 2402, and a flexible and deformable outer sheath 2404 located adjacent the pointed distal tip 2402. The base 2405 of the tissue engaging structure 2400 is made of a flexible material that does not permanently deform as the pointed distal tip 2402 is withdrawn therethrough. The distal tip 2402 and outer sheath 2404 extend outwardly from an anastomosis ring 2406, to engage a patient's tissue portion 2407. The outer diameter of the distal tip 2402 is wider than the inner diameter of the outer sheath 2404. The outer sheath 2404 is made of a material that permanently deforms as the distal tip 2402 is withdrawn through the outer sheath 2404, towards the anastomosis implant 2406. Materials for the outer sheath 2404 can be a shape memory material such as, for example, nitinol. The distal tip 2402 is made of a material sufficient to cause the outer shaft 2404 to permanently deform (as shown in Stage “3”) as the distal tip 2402 is withdrawn through the outer shaft 2404. Stage “1” shows the device 2400 prior to insertion through a tissue portion 2407, with the distal tip 2402 shown in an extended position fully through the outer sheath 2404, such that there is no deformation of the outer sheath 2404. Stage “2” shows the distal tip 2402 and outer sheath 2404 inserted through a tissue portion 2407. Stage “3” shows the device 2400 with the distal tip 2402 (not shown in stage “3”) entirely retracted through the outer sheath 2404, thereby permanently deforming the outer sheath 2404 and forcing it radially outward. As a result of the deformation of the outer shaft 2404, the width of the now deformed outer sheath is larger than the hole in the tissue portion 2407 caused by penetration of the tissue engaging structure 2400, such that a mechanical fit is formed with the tissue 2407 between the anastomosis ring 2406 and the deformed outer sheath 2404 thus anchoring or securing the anastomosis ring 2406 in place. The embodiment of FIG. 50 may be used with the devices and procedures set forth herein, or any other device or procedure that may facilitate extension of the distal tip 2402 and outer sheath 2404, and retraction of the distal tip 2402 to deform the outer sheath 2404.

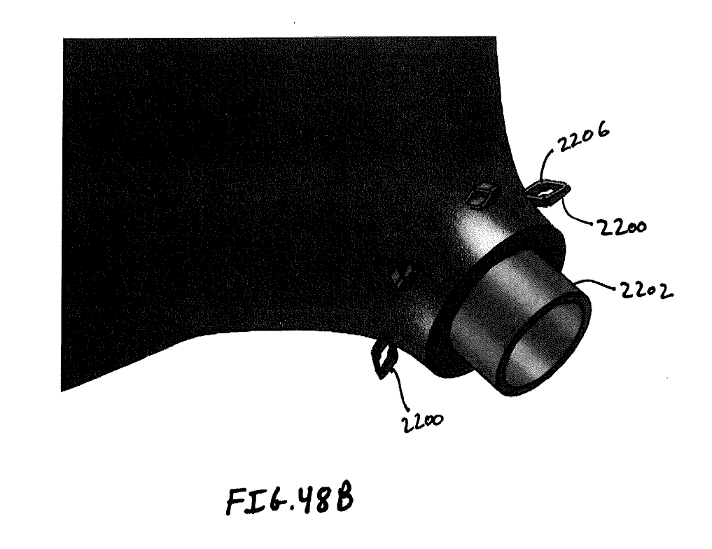

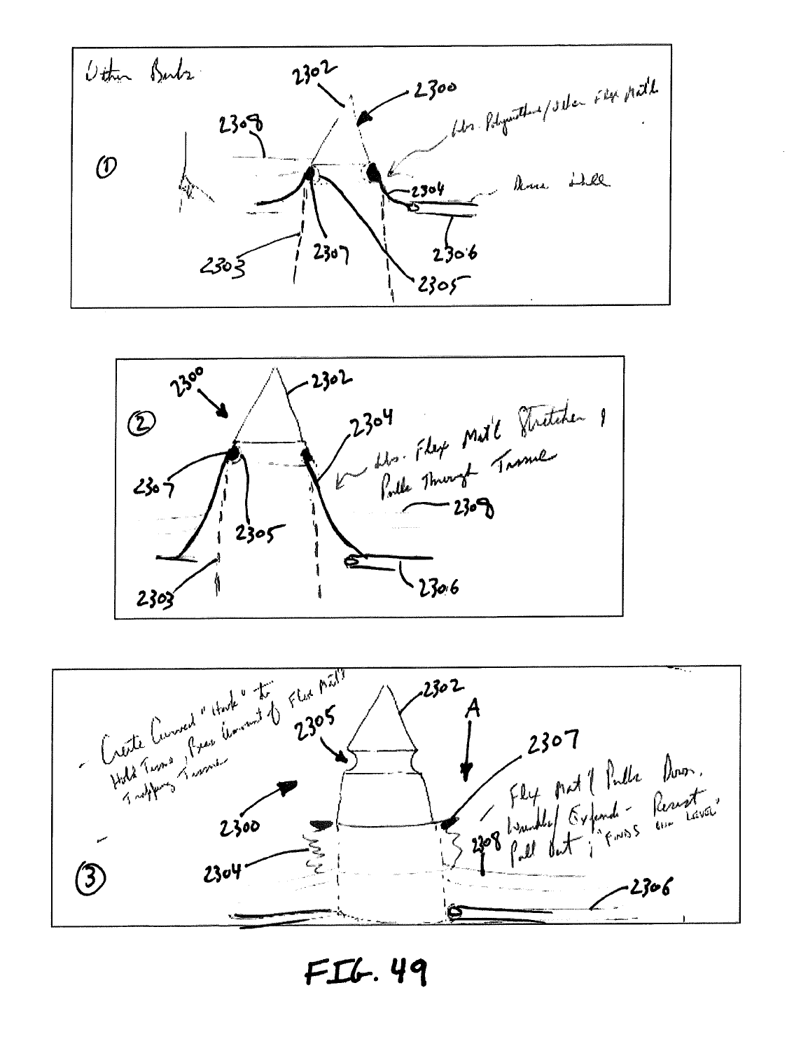

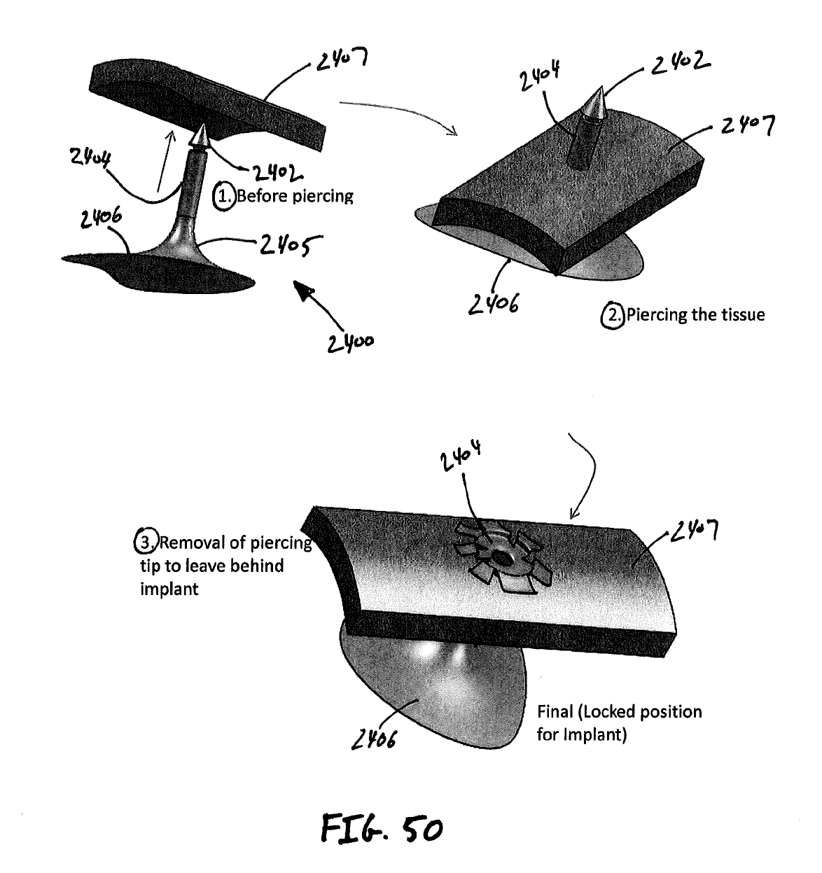

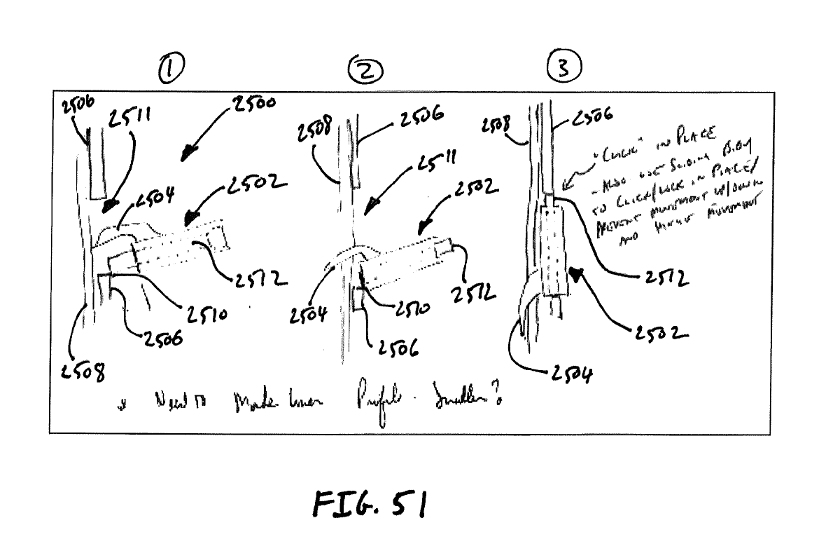

FIG. 51 depicts a further alternative embodiment of an anastomosis device, shown in various stages (1-3) of deployment. The device 2500 includes a pivoting tissue engaging structure 2502 with a protruding tooth 2504 that is adapted to pierce a patient's tissue 2508 during deployment. The tissue engaging structure 2502 is pivotally mounted to an anastomosis implant 2506 by a hinge 2510. In stage “1,” the tissue engaging structure 2502 and tooth 2504 are disposed within the outer diameter of the anastomosis implant 2506, while the tooth 2504 remains unengaged with the tissue 2508. In stage “2,” the tissue engaging structure 2502 is pivoted about the hinge 2510, such that the tooth 2504 is disposed through an aperture 2511 in the sidewall of the implant 2506 and pierces the tissue 2508, in a partially deployed position. Finally, in stage “3,” the tissue engaging structure 2502 is fully deployed, such that it is virtually parallel with the remainder of the anastomosis implant 2506, and the tooth 2504 has fully penetrated the tissue 2508, thereby securing a portion of the tissue 2508 between the tooth 2504 and the anastomosis implant 2506. The tissue engaging structure 2502 may optionally include a locking engagement mechanism 2512, located opposite the hinge 2510, to secure or lock it in the deployed position via a friction or mechanical fit with the anastomosis implant 2506. The embodiment of FIG. 51 may be used with the insertion devices and procedures disclosed herein, or any other device or procedure that may facilitate deployment of the tissue engaging structures 2502.

FIG. 52A depicts a further alternative embodiment of an anastomosis device 2600, shown in various stages (1-2) of deployment. The device of FIG. 52A is similar to the device 2500 of FIG. 51, and is operable in a similar manner. The device 2600 includes a pivoting tissue engaging structure 2602 with a protruding tooth 2604 that is adapted to pierce a patient's tissue (not shown) during deployment. The tissue engaging structure 2602 is pivotally mounted to an anastomosis implant ring 2606 (a first and a second implant ring may be used) by a hinge 2610. In stage “1,” the tissue engaging structure 2602 and tooth 2604 are disposed in an un-deployed position, within the outer diameter of the anastomosis implant ring 2606, while the tooth 2604 remains unengaged with the tissue. In stage “2,” the tissue engaging structure 2602 is fully deployed, such that it is virtually parallel with the remainder of the anastomosis implant ring 2606, and the tooth 2604 would fully penetrate adjacent tissue, thereby securing the anastomosis implant ring 2606 in place. The tissue engaging structures 2602 may optionally include a locking engagement mechanism 2612, located opposite the hinge 2610, to lock them in the deployed position via a friction or mechanical fit with the anastomosis implant ring 2606. After anchoring in tissue portions, the anastomosis implant rings 2606 may be brought into contact with each other and connected using connecting structures that may be integral to each implant ring 2606.

FIG. 52B provides a partially exploded view of the anastomosis device 2600 of FIG. 52A, whereby the tissue engaging structure 2602 is disassembled from the remainder of the anastomosis implant ring 2606. The embodiment of FIGS. 52A and 52 B may be used with the insertion devices and procedures disclosed herein, or any other device or procedure that may facilitate deployment of the tissue engaging structures 2602 and joining of the implant rings 2606 together.

FIG. 53A depicts a further alternative embodiment of an anastomosis device, shown in an un-deployed state. The device includes an anastomosis ring 2700 comprising an implant ring 2702 and a deployer cam ring 2704 having a plurality of rounded indentations 2706 on its inner surface. A plurality of tissue engaging structures or hooks 2708 are pivotably mounted on the implant ring 2702 such that the tissue engaging structures 2708 are deployable between a retracted or un-deployed position and a deployed position. As can be seen in FIG. 53B, in the un-deployed or retraced state, the tissue engaging structures 2708 are each disposed within a respective indentation 2706, so as not to extend outside the anastomosis ring 2700 and engage tissue. Each tissue engaging structure 2708 is attached to the implant ring 2702 at the same end, such that their tissue piercing distal ends 2709 all point in the same direction. The tissue engaging structures 2708 are adapted to pivot inwardly upon rotation of the deployer cam ring 2704, during deployment.

FIG. 53B depicts the anastomosis ring 2700 in the deployed state. To deploy the tissue engaging structures 2708, the deployer cam ring 2704 is rotated counter-clockwise relative to the implant ring 2704 or the implant ring 2704 is rotated clockwise relative to the deployer cam ring 2704 such that the cam surfaces 2710 located between adjacent rounded indentations 2706 act on the inner surfaces of the engaging structures 2708 thereby pivoting the engaging structures 2708 inwardly through adjacent tissue (not shown), to engage a bodily vessel. The embodiment of FIGS. 53A and 53B may be used with the insertion devices and procedures disclosed herein, or any other device or procedure that may facilitate deployment of the tissue engaging structures 2708.

FIG. 54A depicts a portion of a further alternative embodiment of an anastomosis device, shown in various stages (1-2) of deployment. The device is part of an anastomosis ring 2800 having a generally flat inner surface 2802, in an un-deployed state, as shown in stage “1” and a corrugated outer surface 2804, which facilitates bending of the device 2800 into a ring shape, as shown partially in stage “2.” The anastomosis ring 2800 also has a deploying wire 2806 to facilitate movement of the anastomosis ring 2800 from the flat position to the ring shape.

FIG. 54B also depicts the anastomosis device of 54A in various stages (1-3) of deployment. The device is part of an anastomosis ring 2800 having a generally flat inner surface 2802, in an un-deployed position, as shown in stage “1” and a corrugated outer surface 2804, which facilitates bending of the device 2800 into a ring shape, as shown in stages “2” and “3.” The anastomosis ring 2800 also has a deploying wire 2806 to facilitate movement of the anastomosis ring 2800 from a flat, un-deployed state (stage “1”) position to a ring shape or deployed state (stages “2” and “3”). Hooks or other tissue engaging structures 2808 may be positioned on the inner surface 2802 to facilitate engagement of tissue when the anastomosis ring 2800 is in the deployed position, as best seen in stages “2” and “3.” The embodiment of FIGS. 54A and 54B may be used with the insertion devices and procedures disclosed herein, or any other device or procedure that may facilitate deployment of the device 2800.

FIG. 55 is a perspective view of a further alternative embodiment of a clamping device, shown in a closed position. The clamping device 2900 comprises a shaft 2902 and pivotally mounted grasping fingers 2904, 2906 that are operable to open and close as necessary via movement of a first grasping finger 2904, while the second grasping finger 2906 remains fixed to the shaft 2902. In the embodiment shown, a wire 2908 is used to operate the grasping finger 2904; however a second shaft or other structure may also be utilized. The clamping device 2900 can be used to clamp the outside circumference of a tissue portion to provide support when deploying tissue securing structures from the inside of a tissue portion.

FIG. 56 is a perspective view of a further alternative embodiment of a clamping device, shown in a various stages (1-2) of articulation. The clamping device 3000 comprises a shaft 3002 having a flexible distal end 3004 that comprises a plurality flexible fingers 3005, and an articulation means 3006 attached to a push-pull wire 3008 that are operable to articulate the distal end 3004 from a straight position (stage “1”) to a curved position (stage “2”). The degree of curvature of the distal end 3004 can be controlled by the amount of push or pull exerted on the articulation means 3006. In use, the clamping device 3000 can be inserted into the body in a straight configuration through, for example, a trocar. When at the site of interest in the body to be clamped, the distal end 3004 can be positioned adjacent to the tissue to be clamped and the articulation means 3006 can be manipulated to move the push-pull wire in a corresponding manner thereby causing the distal end 3004 to curve around and clamp the tissue therein. The clamping device 3000 can be used to clamp the outside circumference of a tissue portion to provide support when deploying tissue securing structures from the inside of a tissue portion.

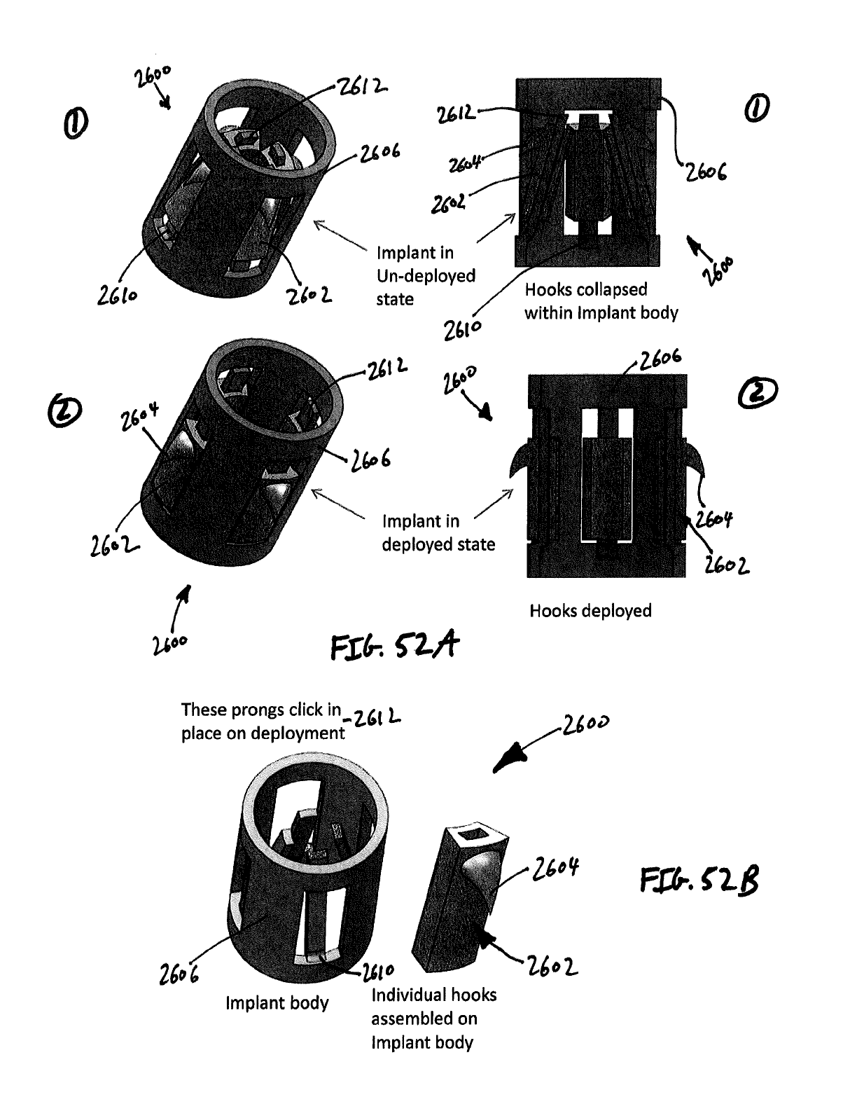

FIG. 57A depicts a further alternative embodiment of an anastomosis device, shown in a deployed state. The device is an anastomosis ring 3100 having hinged or flexible tissue engaging structures 3102. The device 3100 may comprise two ring portions, where each ring portion includes tissue engaging structures 3102 oriented in the same axial direction, as shown in FIG. 57A, or may have two sets of opposing tissue engaging structures 3102, as discussed below. To deploy the tissue engaging structures 3102, a cylindrical sleeve can be inserted into the interior of the anastomosis ring 3100. The cylindrical sleeve can deploy all of the tissue engaging structures 3102 at once or a stepped or cut sleeve 3106 may be used to deploy the tissue engaging structures 3102 at varying degrees at different stages of sleeve insertion.

FIG. 57B depicts another alternative embodiment an anastomosis device similar to that of FIG. 57A. The anastomosis ring 3100 has two sets of opposing, tissue engaging structures 3102 that are adapted to engage opposing tissue portions and retain the tissue portions adjacent each other when deployed. The anastomosis ring 3100 may be deployed using a sheathed device, or any of the methods and devices disclosed herein.

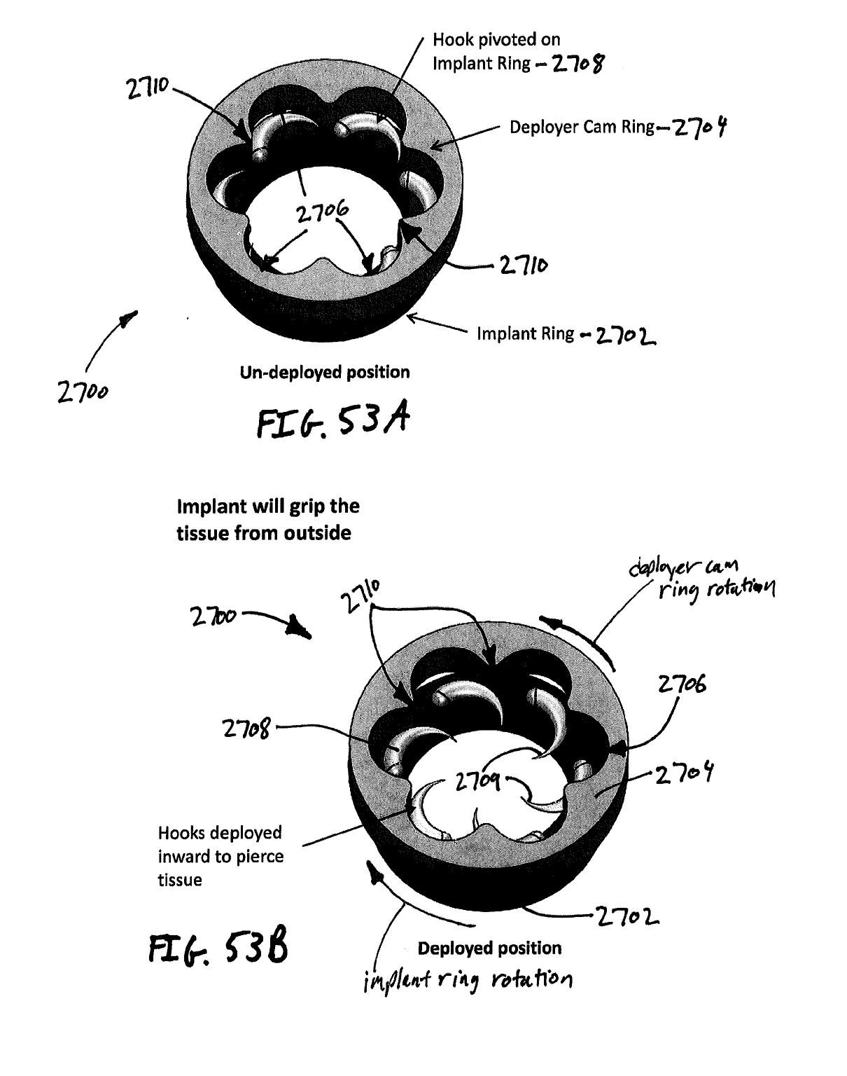

FIG. 58 depicts a further alternative embodiment of a tissue engaging structure 3200 in various stages (1-2) of deployment. The structure includes a set of retractable barbs 3202 disposed therein. Stage “1” shows the tissue engaging structure 3200 with the barbs 3202 in an un-deployed position. Stage “2” shows the tissue engaging structure 3200 with the barbs 3202 in a deployed position. In operation, the tissue engaging structure 3200 may be inserted pointed end first into a desired tissue location. The barbs 3202 may then be deployed from the interior of the structure and the device may be retracted to cause engagement of the barbs 3202 with adjacent tissue.

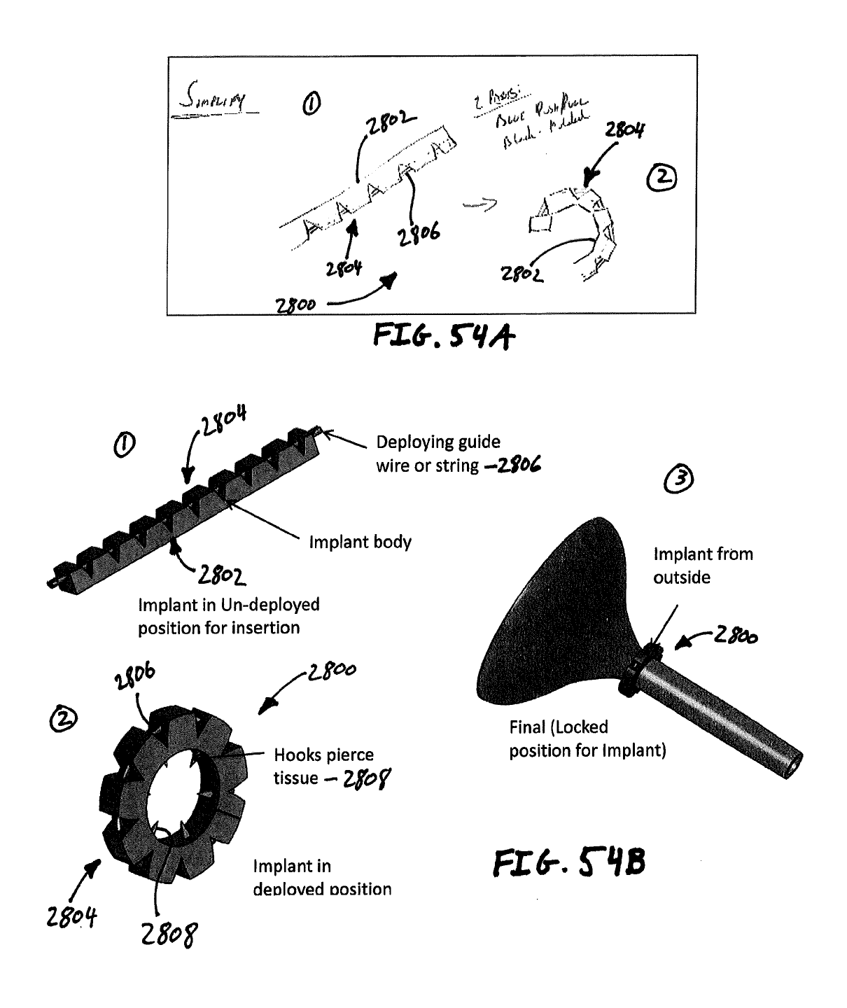

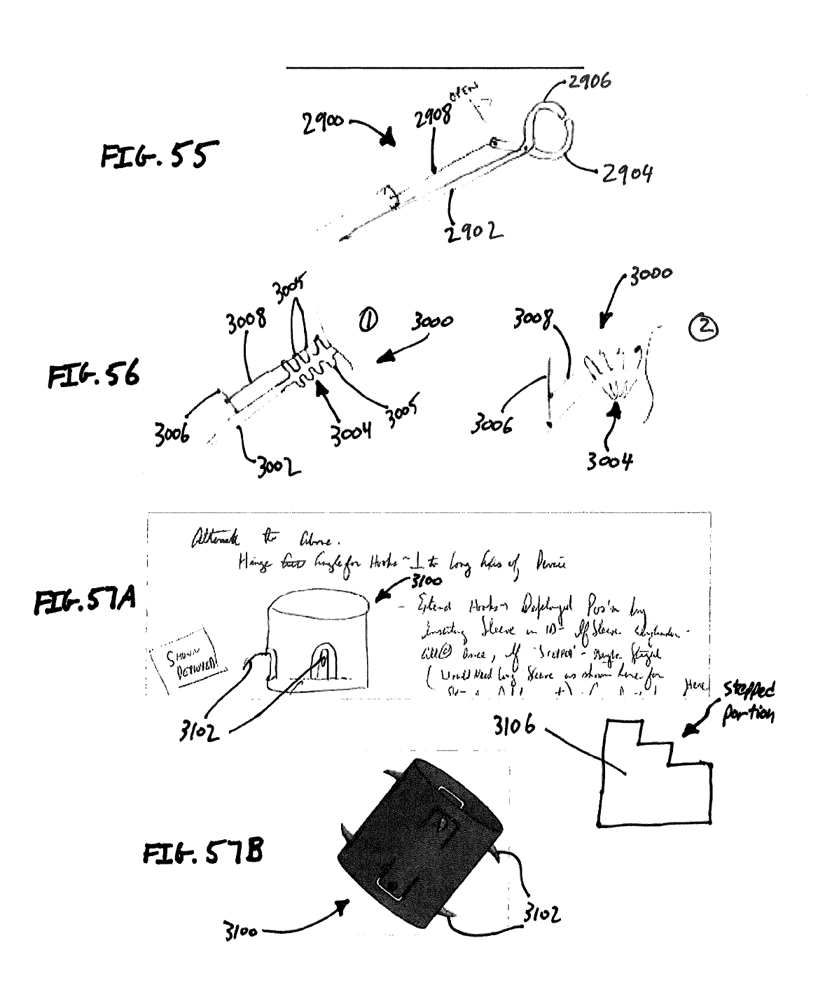

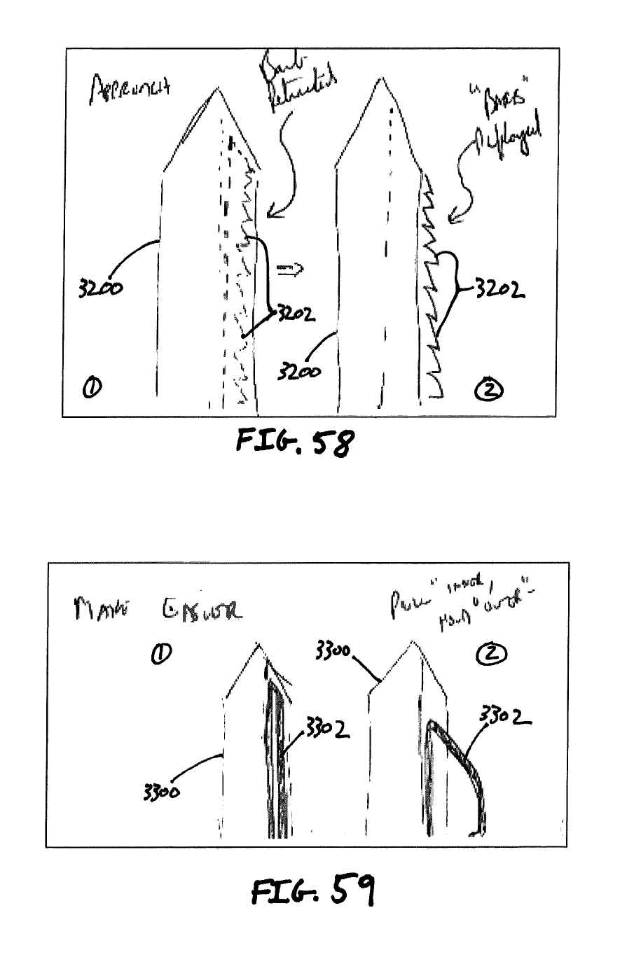

FIG. 59 depicts another alternative embodiment of a tissue engaging structure 3300 in various stages (1-2) of deployment. The tissue engaging structure 3300 includes at least one retractable tooth 3302, or other tissue piercing structure disposed therein. Stage “1” shows the tissue engaging structure 3300 with the tooth 3302 in an un-deployed position. Stage “2” shows the tissue engaging structure 3300 with the tooth 3302 in a deployed position. In operation, the tissue engaging structure 3300 may be inserted pointed end first into a desired tissue location. The tooth 3302 may then be deployed and the device may be retracted to cause engagement of the tooth 3302 and adjacent tissue.

FIG. 60A depicts a further alternative embodiment of an anastomosis device, shown in various stages (1-2) of deployment. The embodiment of FIGS. 60A-60C is similar to that of FIGS. 53A and 53B. The device shown is an anastomosis ring 3400, having deployable tissue engaging structures 3402 pivotally mounted therein. The tissue engaging structures 3402 are deployable in a tangential direction through the outer surface of the ring 3400. Stage “1” shows the ring 3400 with tissue engaging structures 3402 in the un-deployed position. Stage “2” shows the ring 3400 having been rotated clockwise and the tissue engaging structures 3402 deployed so as to engage tissue located adjacent the ring 3400.

FIG. 60B depicts the anastomosis device of FIG. 60A from the side in various stages (1-2) of deployment. Stage “1” shows the tissue engaging structures 3402 in a retracted state, while stage “2” shows the tissue engaging structures 3402 deployed and slanted with respect to the top of the anastomosis ring 3400 to facilitate engagement and retention of the tissue in a desired axial direction.

FIG. 60C is another depiction of the anastomosis device of FIG. 60A, shown in a deployed position. FIG. 60C also depicts an outer clamp 3406, which is used in connection with the anastomosis ring 3400 to facilitate engagement of the tissue engaging structures 3402 in adjacent tissue. The clamp 3406 is preferably applied to the outer surface of tissue, while the anastomosis ring 3400 is disposed within an area defined by the tissue. The embodiment of FIGS. 60A-60C may be used with the insertion devices and procedures disclosed herein, or any other device or procedure that may facilitate deployment of the tissue engaging structures 3402.

FIG. 61 depicts a further alternative embodiment of an anastomosis device, shown in an un-deployed state. The device of FIG. 61 is an anastomosis ring 3500 similar to that discussed above in FIGS. 60A-C, and has retractable tissue hooks 3502. The hooks 3502 are mounted on flexible tissue engaging structures 3505, which are pivotable with respect to the remainder of the anastomosis ring 3500 to deploy the hooks 3502. The hooks 3502 are retained within the outer circumference of the anastomosis ring 3500 in a retracted state and pivoted outward, such that the hooks 3502 extend outwardly of the anastomosis ring 3500 upon deployment. The tissue engaging structures 3505 and hence, the hooks 3502, are deployed by rotating cams 3510 disposed on the inner wall of the anastomosis ring 3500. The anastomosis ring 3500 of FIG. 61 may be used on the interior of a tissue portion such that the hooks 3502 when deployed engage the interior wall of the tissue portion or the anastomosis ring 3500 may be used on the exterior of a tissue portion such that the hooks 3502 when deployed engage the exterior surface of the tissue portion. The embodiment of FIG. 61 may be used with the insertion devices and procedures disclosed herein, or any other device or procedure that may facilitate deployment of the tissue engaging structures 3505 and hence deployment of the hooks 3502.

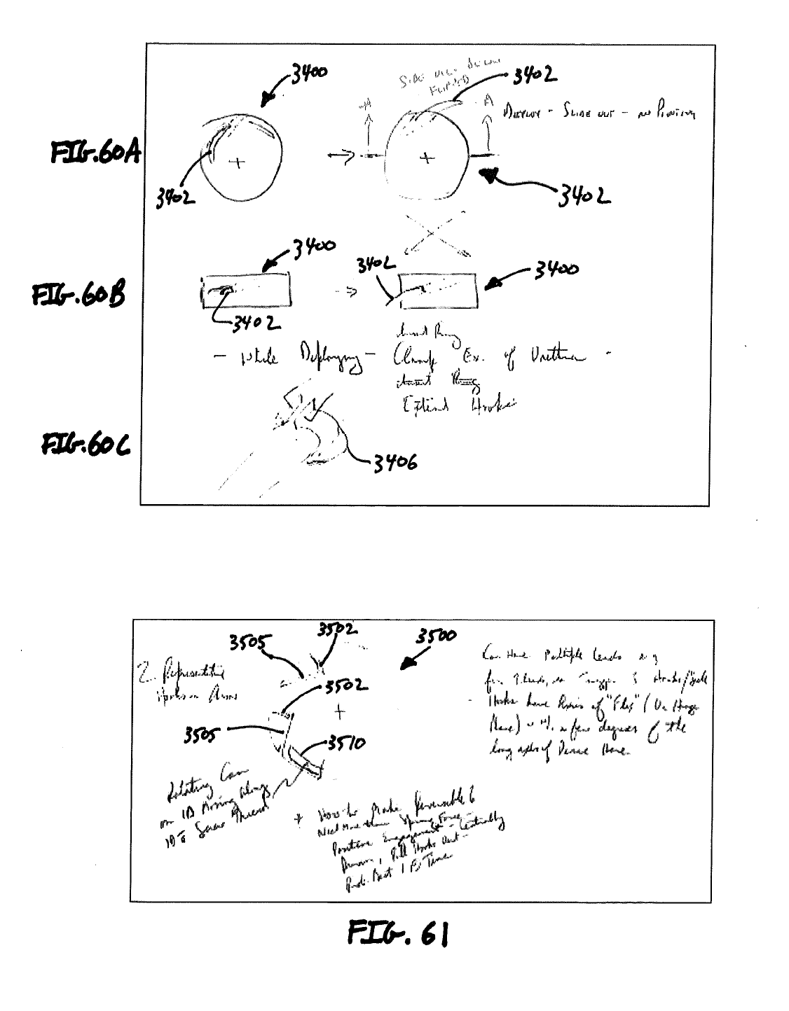

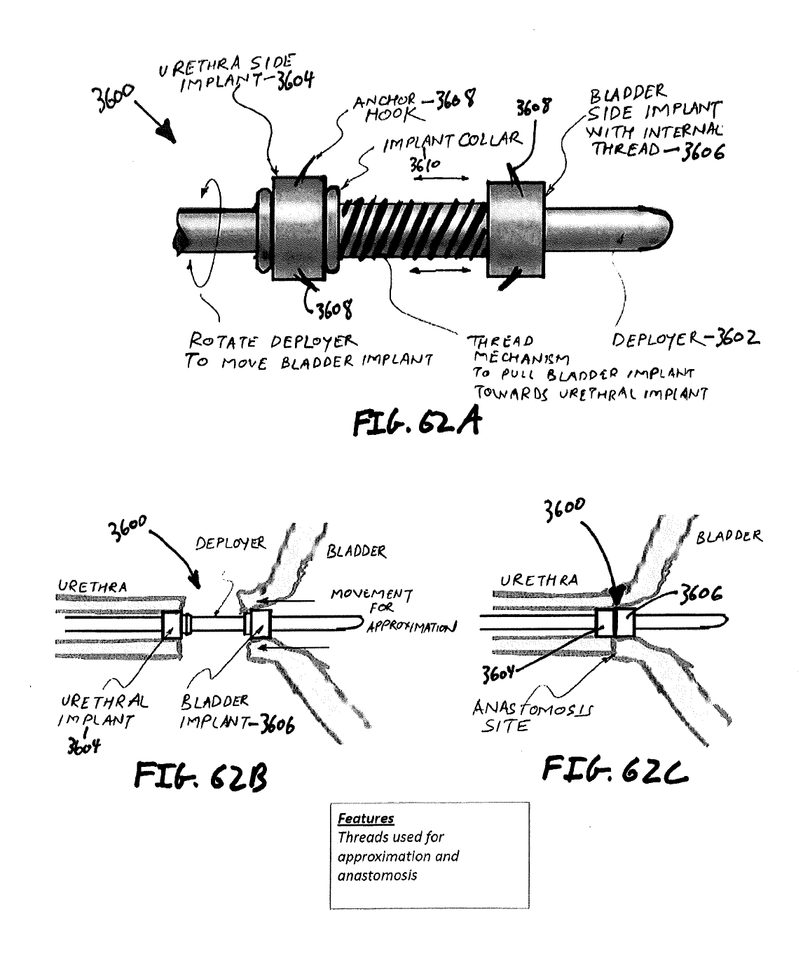

FIG. 62A depicts a further alternative embodiment of an anastomosis device, shown in a deployed, but un-retracted state. The anastomosis device 3600 comprises a deployer 3602, first and second rings 3604, 3606, each having tissue engaging structures or hooks 3608 for engaging adjacent tissue. The deployer 3602 includes a threaded mechanism 3610 used to draw the first and second rings 3604, 3606 toward each other, thereby facilitating the hooks 3608 engagement with adjacent tissue and anastomosis of two tissue portions by joining the first and second rings 3604, 3606 together.

As can be seen in FIG. 62B, during delivery, the first ring 3604 is delivered to the first tissue portion (here, the urethra) and the second ring 3606 is delivered to the second tissue portion (here, the bladder). Once the rings 3604, 3606 are in the desired tissue locations, the tissue engaging structures 3608 are deployed and engage the tissue.

As depicted in FIG. 62C, after the tissue engaging structures 3608 are engaged with their respective tissue portions, the first and second rings 3604, 3606, are drawn toward each other through activation of the threaded deployer 3602 thereby also drawing the first and second tissue portions toward each other. The first and second rings 3604, 3606 are drawn toward each other until they contact each other causing the implant collar 3610 on the first ring 3604 engage a corresponding structure on the second ring 3606 thereby joining the first and second rings together and completing the anastomosis. The embodiment of FIGS. 62A-62C may be used with the insertion devices and procedures disclosed herein, or any other device or procedure that may facilitate deployment of the tissue engaging structures 3608 and joining of the first and second rings 3604, 3606 with each other.

FIG. 63A depicts a further alternative embodiment of an anastomosis device in various stages (1-2) of deployment. The device comprises a spring loaded clip 3700, having opposing first and second sets of tissue engaging structures or teeth 3702, 3704, and a flexible shape memory spring-like material 3706 joining the teeth 3704.

As can be seen in FIG. 63B, to facilitate anastomosis, the first teeth 3704 are inserted into adjacent first tissue (here, the bladder), and the shape memory material 3706 is then stretched from its normal state to allow the second teeth 3702 to be inserted into and engaged with second tissue (here, the urethra) that is spaced from that engaged by the first teeth 3704. As depicted in FIG. 63C, once all of the tissue engaging structures 3702, 3704 are in place and engaged with tissue, the shape memory material 3706 returns to its pre-stretched state, pulling the adjacent tissue portions to be joined into contact with each other, thereby completing the anastomosis.

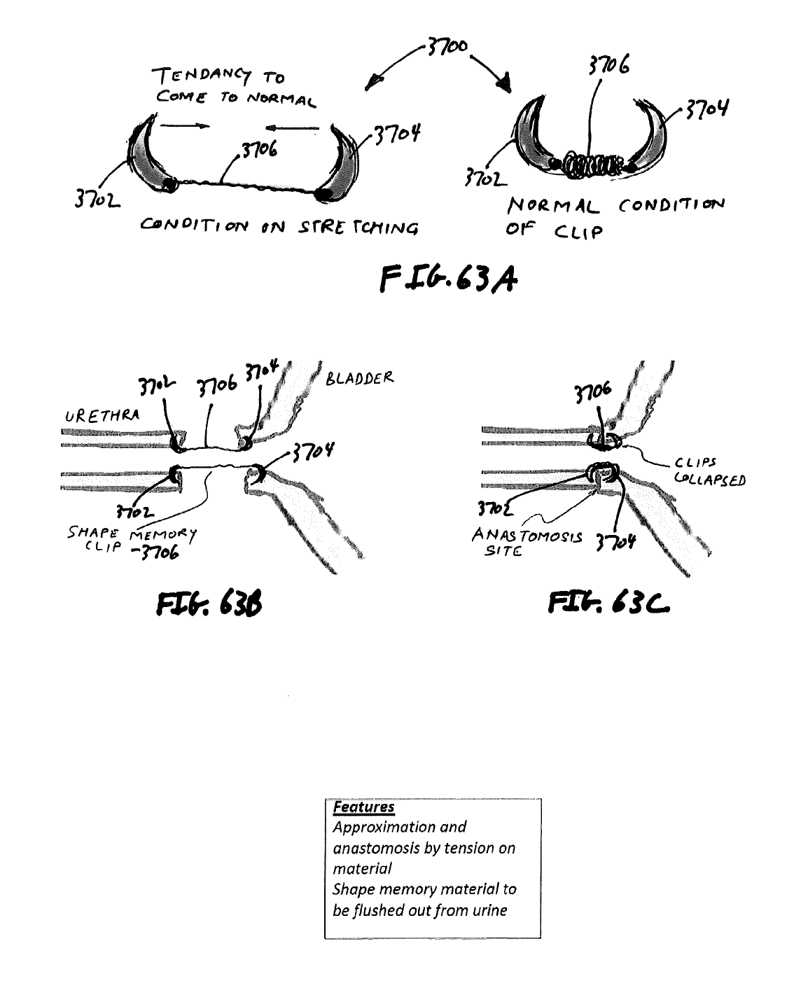

FIG. 64A depicts a further alternative embodiment of an anastomosis device, shown in a deployed, but un-retracted state. The anastomosis device 3800 comprises a deployer 3802, first and second rings 3604, 3606, each having tissue engaging structures 3808 for engaging adjacent tissue. The first and second rings 3804, 3806 include corresponding coupling structures or means 3810 that engage each other when the rings 3804, 3806 are brought into contact with each other, thereby joining the rings together. The tissue engaging structures 3806 are made from a shape memory material such as nitinol and act about a live hinge to transition from an un-deployed state during device delivery to a deployed state when the first and second rings 3804, 3806 are in place adjacent first and second tissue portions.

As can be seen in FIG. 64B, during delivery, the first ring 3804 is delivered to the first tissue portion (here, the urethra) and the second ring 3806 is delivered to the second tissue portion (here, the bladder). Once the rings 3804, 3806 are in the desired tissue locations, the shape memory characteristics of the tissue engaging structures 3808 (namely, the heating up of the tissue engaging structures 3808 by body heat) causes tissue engaging structures 3808 to return to their pre-set deployed state thereby engaging adjacent tissue.

As depicted in FIGS. 64C and 64D, after the tissue engaging structures 3808 are engaged with their respective tissue portions, the first and second rings 3804, 3806, are drawn toward each other through activation of the deployer 3802 thereby also drawing the first and second tissue portions toward each other. The first and second rings 3804, 3806 are drawn toward each other until they contact each other causing the coupling structures 3810 on the first and second rings 3804, 3806 to engage each other thereby joining the first and second rings 3804, 3806 together and completing the anastomosis. The embodiment of FIGS. 64A-64D may be used with the insertion devices and procedures disclosed herein, or any other device or procedure that may facilitate delivery of the device 3800 and joining of the first and second rings 3804, 3806 with each other.

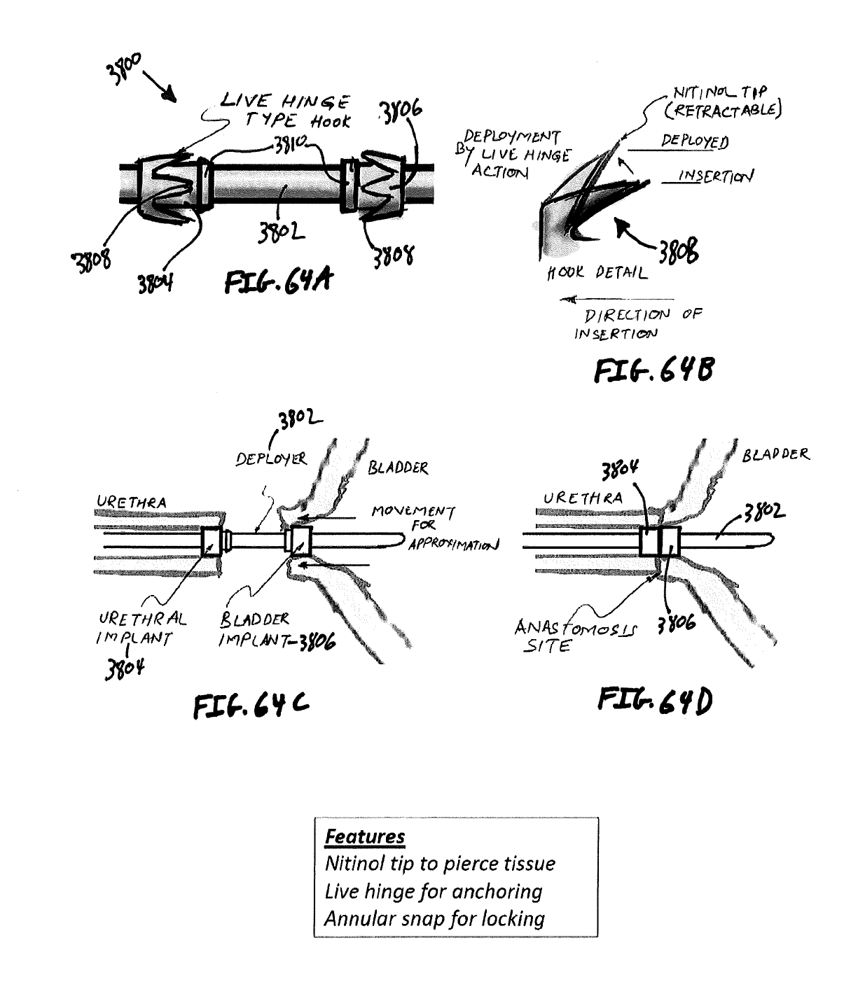

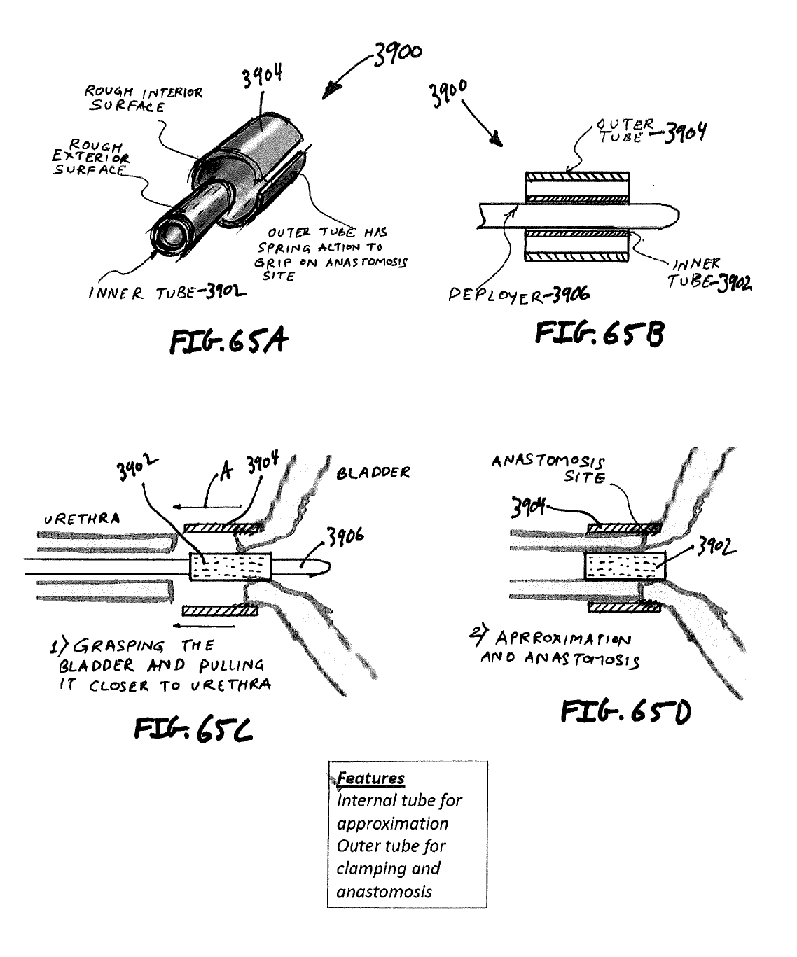

FIG. 65A depicts a further alternative embodiment of an anastomosis device. The device is an anastomosis clamp 3900, having inner and outer tubular sleeves 3902, 3904, which are adapted to engage tissue there between. The exterior surface of the inner tube 3902 and the interior surface of the outer tube 3904 may be roughened or may include engagement structures, such as, for example, teeth, barbs or ridges, to facilitate grasping of body tissue.

As shown in FIG. 65B, at least one (preferably both) of the inner and outer tubes 3902, 3904 may be mounted on a deployer 3906, for insertion into a patient. As depicted in FIG. 65C, the inner tube 3902 is mounted on a deployer 3906 and inserted into a desired tissue site. Once the inner tube 3902 is positioned at the desired first tissue portion location, the outer tube 3904 is then inserted radially outward of the tissue, thereby retaining the first tissue portion (here, the bladder) to be anastomosed between the tubes 3902, 3904. Once, the first tissue portion is grasped between the inner and outer tubes 3902, 3904, with the aid if the deployer 3906, the anastomosis device 3900 is drawn toward the second tissue portion (here, the urethra) to be anastomosed as depicted by the arrow A in FIG. 65C. The inner and outer tubes 3902, 3904 are then inserted onto the second tissue portion thereby completing the anastomosis as depicted in FIG. 65D.

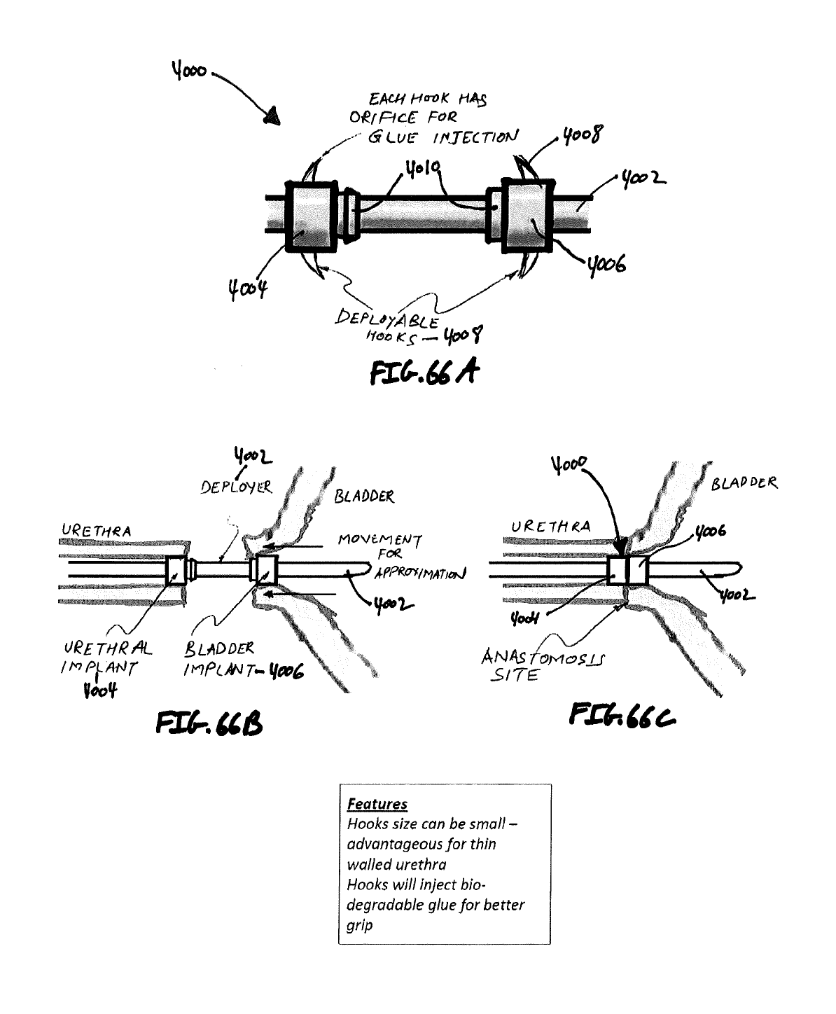

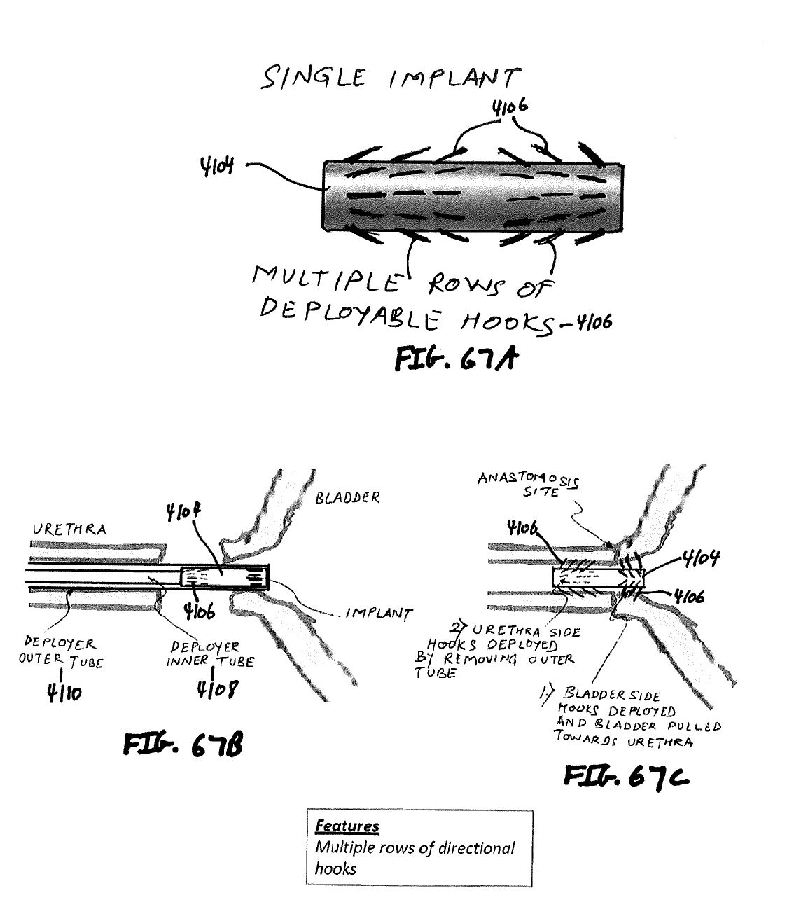

FIG. 66A depicts a further alternative embodiment of an anastomosis device. The anastomosis device 4000 comprises a deployer 4002, first and second rings 4004, 4006 each having tissue engaging structures 4008 for engaging adjacent tissue. The first and second rings 4004, 4006 include corresponding coupling structures or means 4010 that engage each other when the rings 4004, 4006 are brought into contact with each other, thereby joining the rings 4004, 4006 together. In this embodiment, the tissue engaging structures 4006 include orifices at their pointed tips in order to inject a biodegradable adhesive or material in order to facilitate anchoring of the structures to body tissue. The tissue engaging structures 4008 are deployable from a retracted to an extended position with the aid of the deployer 4002.

As can be seen in FIG. 66B, during delivery, the first ring 4004 is delivered to the first tissue portion (here, the urethra) and the second ring 4006 is delivered to the second tissue portion (here, the bladder). Once the rings 4004, 4006 are in the desired tissue locations, the tissue engaging structures 4008 are deployed through activation of the deployer 4002.

As depicted in FIG. 66C, after the tissue engaging structures 4008 are engaged with their respective tissue portions, the first and second rings 4004, 4006, are drawn toward each other through activation of the deployer 4002 thereby also drawing the first and second tissue portions toward each other. The first and second rings 4004, 4006 are drawn toward each other until they contact each other causing the coupling structures 4010 on the first and second rings 4004, 4006 to engage each other thereby joining the first and second rings 4004, 4006 together and completing the anastomosis. At any point after the tissue engaging structures 4008 are engaged and seated within body tissue, the adhesive can be injected into the tissue through the orifices at the tips of the tissue engaging structures 4008 in order to better secure the first and second rings 4004, 4006 to the tissue. The embodiment of FIGS. 66A-66C may be used with the insertion devices and procedures disclosed herein, or any other device or procedure that may facilitate delivery of the device 4000, deployment of the tissue engaging structures 4008, and joining of the first and second rings 4004, 4006 with each other.

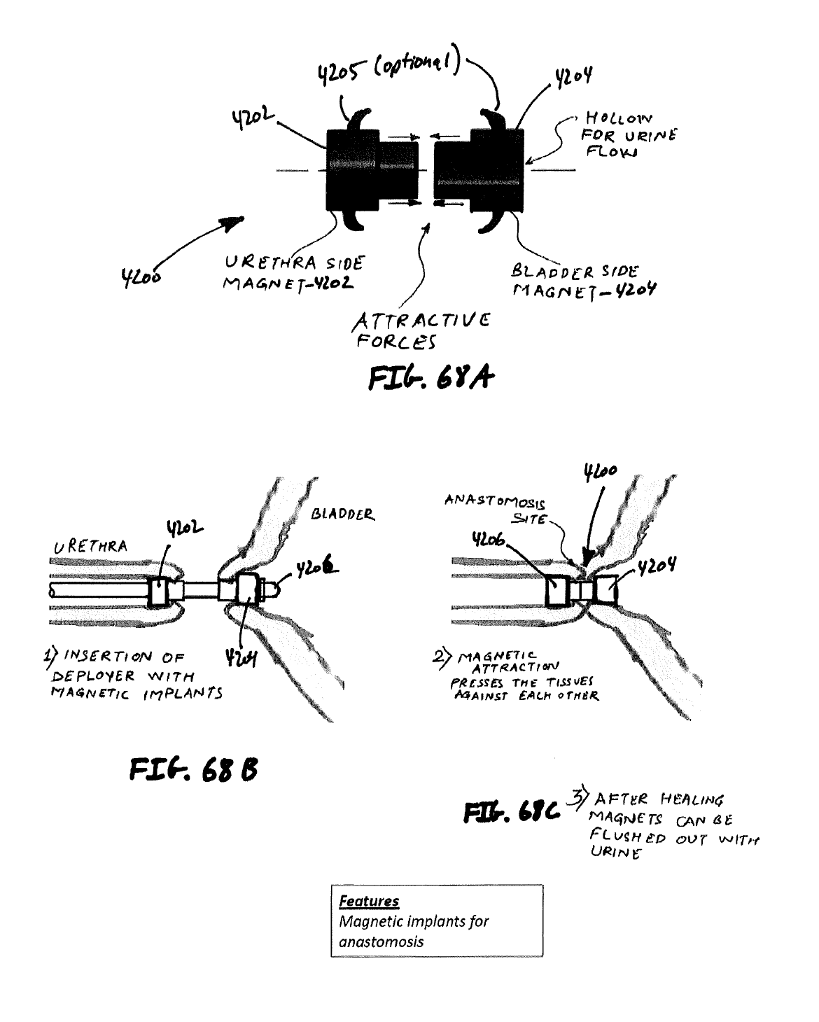

FIG. 67A depicts a further alternative embodiment of an anastomosis device. The anastomosis device 4100 comprises a deployer 4102 (FIG. 67B), and an anastomosis cylinder 4104. The anastomosis cylinder 4104 is provided with two sets of opposing tissue engaging structures or hooks 4106, for engaging two respective adjacent tissue portions. The deployer 4102 includes an inner tube 4108 to hold the anastomosis cylinder 4104 and an outer tube 4110 that acts as a sheath to cover the opposing tissue engaging structures 4106 during device delivery.

As can be seen in FIG. 67B, during device delivery, the anastomosis cylinder 4104 may be retained within the outer tube 4110 of the deployer 4102, thereby biasing the hooks 4106 inwardly against the anastomosis cylinder 4104. With the hooks 4106 in a retracted state and covered by the outer tube 4110, insertion into a desired tissue location is made easier and safer, due to the reduction of risk of unintentionally engaging tissue.

As depicted in FIG. 67C, once the anastomosis cylinder 4104 is disposed at a desired location, the outer tube 4110 of the deployer 4102 is at least partially retracted to expose a first set of hooks 4106 in order to allow the first set of hooks 4106 to engage a first tissue portion (here, the bladder). After the first set of hooks 4106 are engaged, the deployer 4102 and hence, the anastomosis cylinder 4104, is partially withdrawn in order to (i) set the first set of hooks 4106 in the first tissue portion, and (ii) pull the first tissue portion into contact with the second tissue portion (here, the urethra). At this point, the outer tube 4110 is further retracted to expose a second set of hooks 4106 for engagement with the second tissue portion, thereby completing the anastomosis Although FIGS. 67A-67C show a unitary cylinder that includes tissue engaging structures for both tissue portions, those skilled in the art would understand that the device can include a first and a second cylinder with each cylinder having tissue engaging structures and where the first cylinder engages a first tissue portion and a second cylinder engages a second tissue portion. After the first and second cylinders are anchored to their respective tissue portions, they can then be brought together and joined to each other to complete the anastomosis using any of the insertion devices and procedures disclosed herein. Moreover, although the tissue engaging structures 4106 in the embodiment of FIGS. 67A-67C are shown as being integral with the anastomosis cylinder 4104, those skilled in the art would understand that the tissue engaging structures could be included on a separate structure that is used in combination with the anastomosis cylinder similar to some of the other embodiments disclose herein (see e.g., FIG. 46).

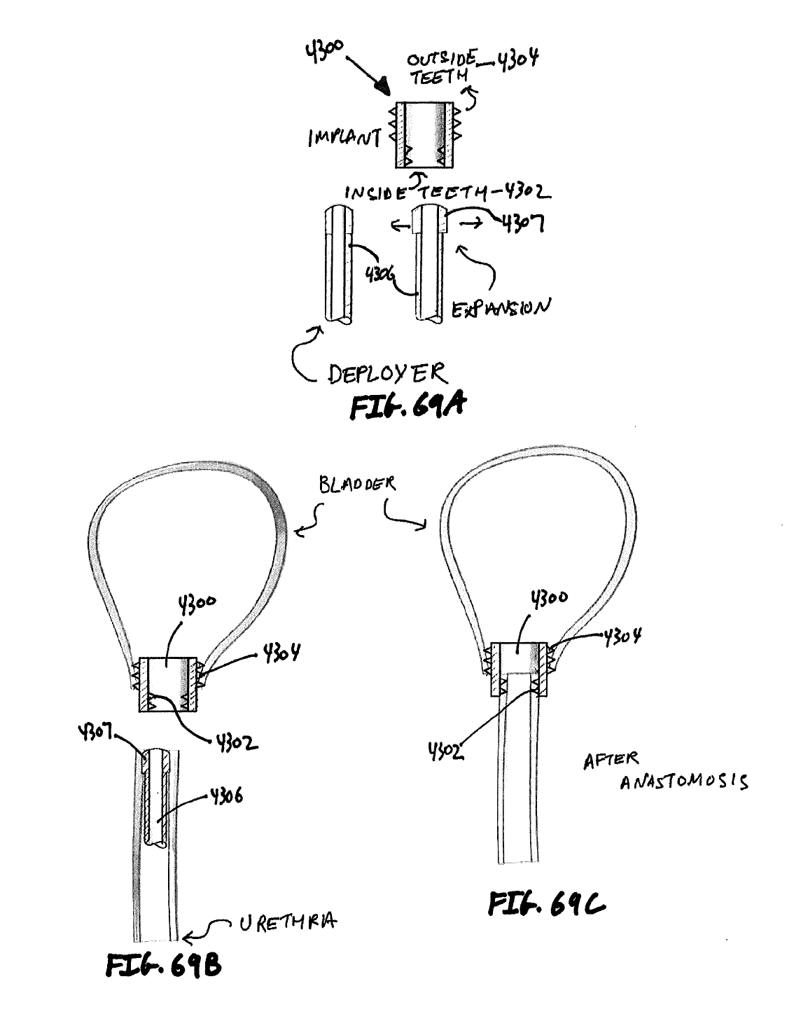

FIG. 68A depicts a further alternative embodiment of an anastomosis device. The anastomosis device 4200 comprises first and second opposing magnetic rings 4202, 4204. Each ring 4202, 4204 may be provided with tissue engaging structures 4205, or there may be a void between the rings 4202, 4204 to compress tissue there between when they are joined. Each ring 4202, 4204 is also preferably hollow to allow fluid, such as urine, to pass therethrough, if necessary.

As can be seen in FIG. 68B, the rings 4202, 4204 may be inserted into a desired tissue area using a deployer 4206. Preferably, the first ring 4202 engages, with the aid of the tissue engaging structures 4205, a first tissue portion (here, the urethra), and the second ring 4204 engages, with the aid of tissue engaging structures 4205, a second tissue portion (here, the bladder) that is spaced from the first tissue portion. The tissue may overlap portions of the first and second rings 4202, 4204 that face each other, in order to provide surface area for clamping together. Once the first and second rings 4202, 4204 have engaged their respective tissue portions, as can be seen in FIG. 68C, the rings 4202, 4204 are brought together and magnetically joined to each other, with portions of the adjacent tissue optionally clamped there between. The compressed tissue is then allowed to naturally heal. The embodiment of FIGS. 68A-68C may be used with the insertion devices and procedures disclosed herein, or any other device or procedure that may facilitate delivery of the device 4200, deployment of the tissue engaging structures 4205, and joining of the first and second rings 4202, 4204 with each other.

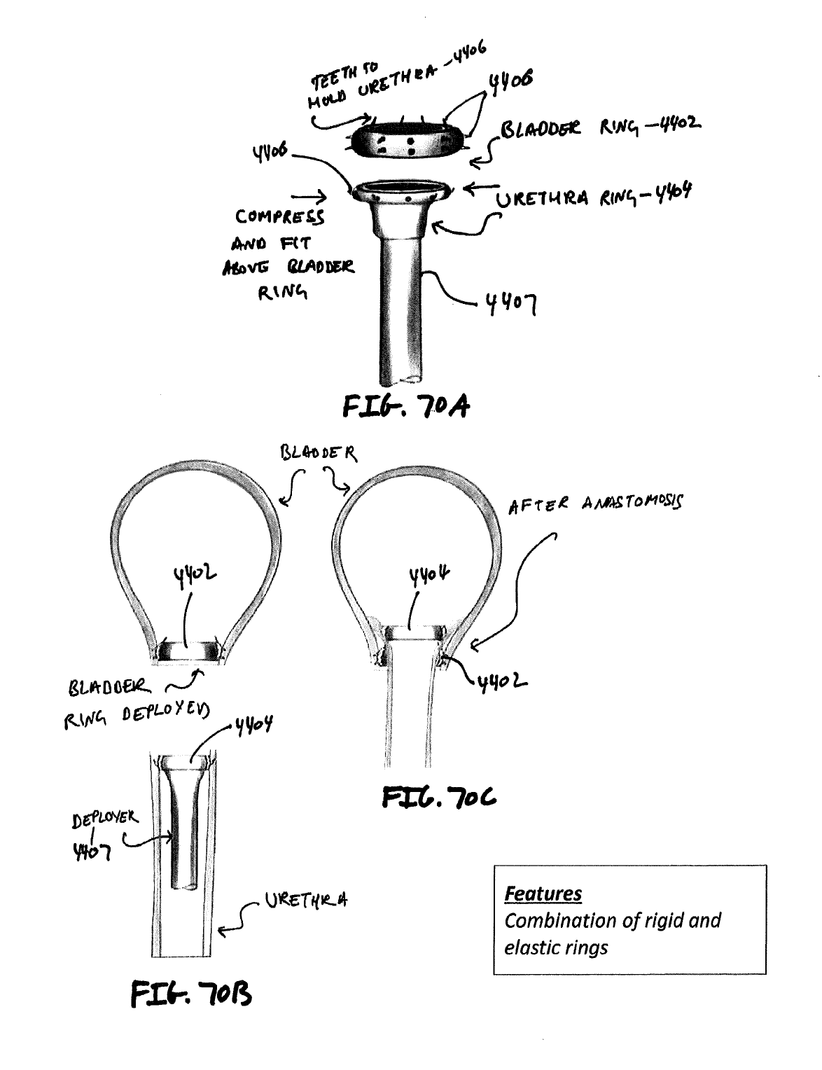

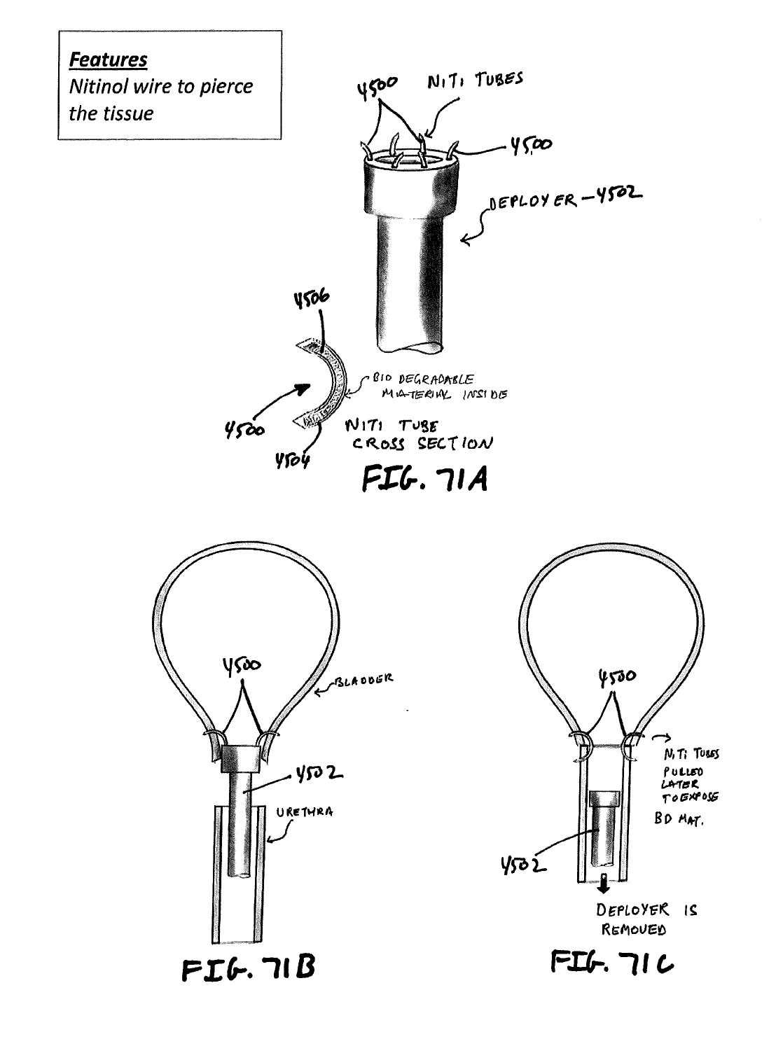

FIG. 69A depicts a further alternative embodiment of an anastomosis device. The device comprises a single anastomosis ring 4300, with inner and outer tissue engaging structures or teeth 4302, 4304. The ring 4300 is inserted via a deployer 4306 that includes an expanding portion 4307 in order to expand at least a portion of the ring 4300 in order to facilitate its fitting around a first tissue portion as can be seen in FIGS. 69B and 69C. Preferably, the ring 4300 is first inserted into a first tissue portion, such that the outer teeth 4302 engage the inner surface of the respective tissue portion. The deployer 4306 may then be used to retract the ring 4300 towards an adjacent tissue portion.

As depicted in FIG. 69C, once the outer teeth 4304 are engaged with the first tissue portion (here, the bladder), the ring 4300 is drawn towards the second tissue portion (here, the urethra) and expanded with the expanding portion 4307 to a diameter that is larger than the outer diameter of the second tissue portion, such that the inner teeth 4302 are spread around the outer surface of the second tissue portion. The ring 4300 is then allowed to collapse onto the second tissue portion, forcing the teeth 4302 to engage the tissue, thereby completing the anastomosis.

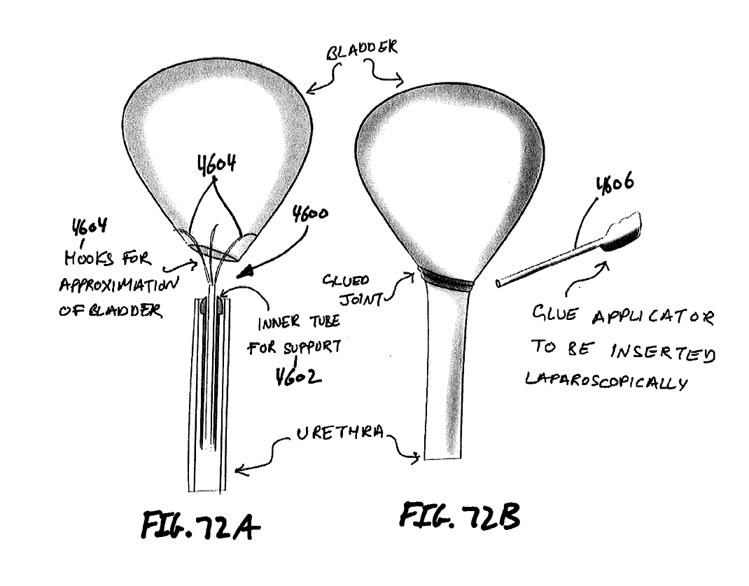

FIG. 70A depicts a further alternative embodiment of an anastomosis device. The device comprises two anastomosis rings 4402 and 4404, each having deployable tissue engaging structures 4406 to engage respective adjacent tissue portions and a deployer 4407.

During operation, as can be seen in FIGS. 70B and 70C, the first ring 4402 is disposed within a vessel or other tissue portion (here, the bladder) with its tissue engaging structures 4406 engaging the inner surface of the tissue portion. The second ring 4404 is narrower than the first ring 4402 and is compressible or elastic, such that it may fit within an inner diameter of the first ring 4402, thereby facilitating passage of the second ring 4404 (and its respective engaged tissue, here, the urethra) through the second ring 4404.

In operation, the tissue engaging structures 4406 of the second ring 4404 engage a desired tissue portion (here, the urethra) and the second ring 4404 is then compressed. Once compressed, the second ring 4404 is passed through the first ring 4402, and then expanded. The first and second rings 4402, 4404 and hence the first and second tissue portions are then drawn together, thereby forming a compression fit or engagement between the rings 4402, 4404 thereby completing the anastomosis.

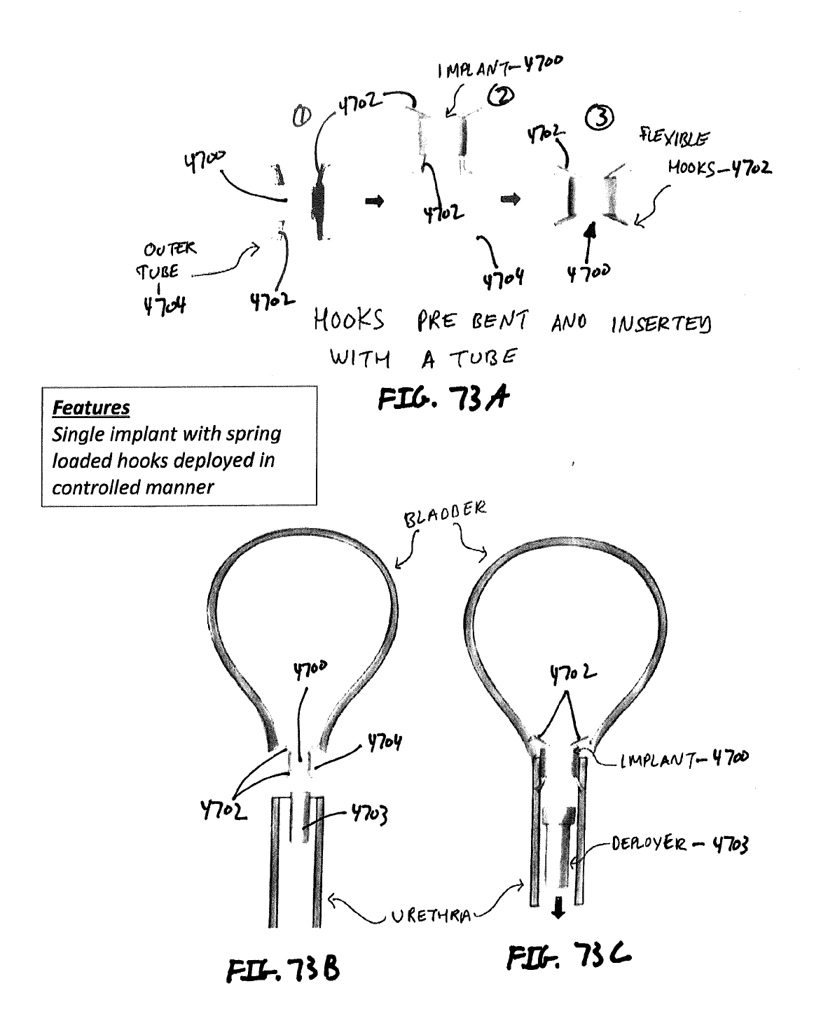

FIG. 71A depicts a further alternative embodiment of an anastomosis device. The device comprises a plurality of tubular tissue engaging structures or hooks 4500 that are inserted into desired tissue portion, using a deployer 4502. Each tissue engaging structure 4500 is made from a hollow shape memory material such as nitinol 4504 with a biodegradable core 4506. As can be seen in FIG. 71B. During insertion, a first end of the tissue engaging structure 4500 extends from the deployer 4502 and is inserted into a first tissue portion (here, the bladder).

As shown in FIG. 71C, once the tissue engaging structures 4500 are secured to the first tissue portion, the first tissue portion is drawn towards the second tissue portion (here, the urethra) and the remainder if each tissue engaging structure 4500 is inserted into the second tissue portion. Because of the shape memory properties of nitinol, the tissue engaging structures 4500 transform to their original “C” shape (shown in FIG. 71C), thereby bringing the two tissue portions into contact with each other, completing the anastomosis. Once installed in the tissue, the outer tube 4506 may be removed, leaving the biodegradable core 4504 to hold the tissue together, which naturally degrades during the healing process.

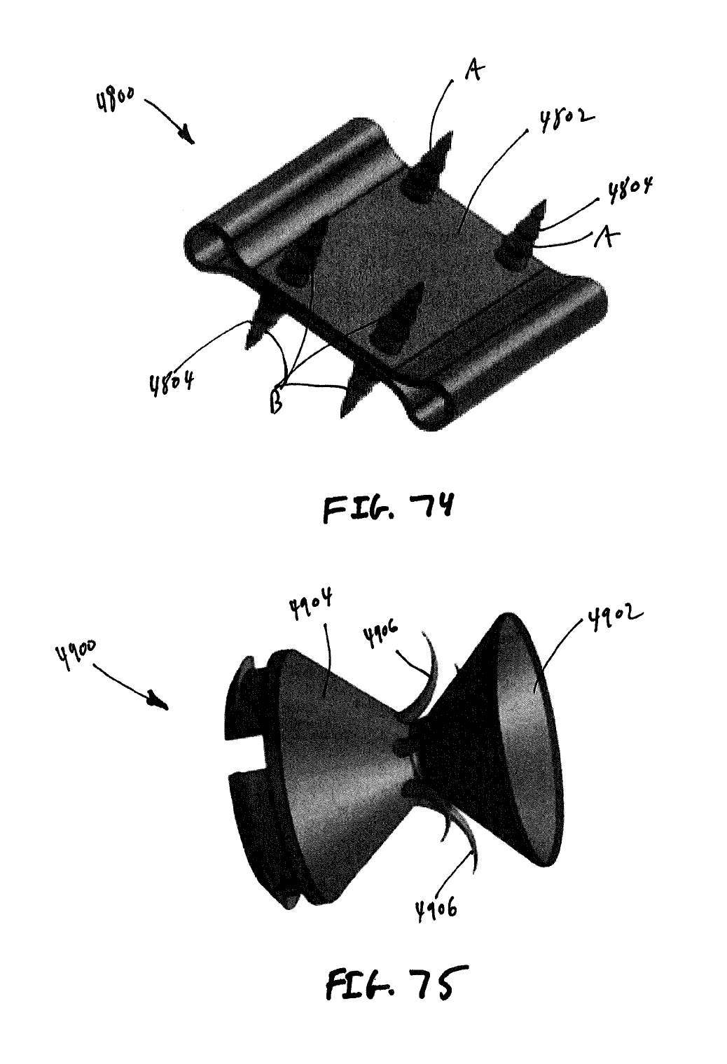

FIG. 72A depicts a further alternative embodiment of an anastomosis device. The device includes an approximation device 4600, having an inner deployer tube 4602 and retractable approximation hooks 4604. The hooks 4604 preferably engage a first tissue portion (here, the bladder) and draw it towards a second tissue portion (here, the urethra).

As can be seen in FIG. 72B, once the first and second tissue portions are drawn together, an adhesive is applied to the outer seam between the respective tissue portions. The adhesive is applied using an applicator 4606 that is laproscopically inserted into the anastomosis site.

FIG. 73A depicts is a further alternative embodiment of an anastomosis device. The device comprises an anastomosis ring 4700 having opposing sets of flexible tissue engaging structures or hooks 4702 pivotably or flexibly mounted therein and a tubular deployer 4703. During deployment, the ring 4700 is initially retained in a sheath 4704 such that it may be positioned in a desired tissue location. As can be seen in FIG. 73B, the sheath 4704 protects body tissue from being damaged by the tissue engaging structures 4702 during delivery.

Referring to FIG. 73A, Stage “1” of the deployment shows the ring 4700 fully disposed within the sheath 4704. The hooks 4702 are preferably biased in a radially outward direction, such that the sheath 4704 serves to hold them radially inward during insertion. Stage “2” shows a first stage of deployment, where the sheath 4704 is partially removed to allow a first set of hooks 4702 to release radially outward, thereby engaging adjacent first tissue portions. Stage “3” shows the ring 4700 with the second set of hooks 4702 released and expanded to engage second tissue portions.

As depicted in FIGS. 73B and 73C, once the ring 4700 is positioned and the first set of hooks 4702 are deployed to engage a first tissue portion (here, the bladder), the ring 4700 and first tissue portion are drawn towards a second tissue portion (here, the urethra). The remaining hooks 4702 are then released by retracting the sheath 4704 further and their bias facilitates their engagement with the second tissue portion. The hooks' 4702 outward bias, which forms the opposing tips of each hook 4702 toward each other, pulls the first and second tissue portions into contact with each other forming a compressive engagement of the two tissue portions, completing the anastomosis.

FIG. 74 depicts a further alternative embodiment of an anastomosis device. The device is an anastomosis ring 4800 that may be deployed using devices and methods discussed herein. The ring 4800 includes a flexible sleeve 4802 that may be inserted into a desired location in a compressed state. The ring also includes barbed tissue engaging structures 4804 extending radially outward from the flexible sleeve 4802 and are adapted to penetrate adjacent tissue portions (tissue engaging structures “A” are adapted to engage first tissue portions and tissue engaging portions “B” are adapted to engage second tissue portions), thereby anchoring the anastomosis ring 4800 in place and joining the first and second tissue portions together. Those skilled in the art would understand that the device can also be a two-piece structure comprising a first flexible sleeve that includes tissue engaging structures “A” and a second flexible sleeve that includes tissue engaging structures “B” where each sleeve can be delivered and anchored separately to its respective tissue portion and the joined together to complete the anastomosis. Because the anastomosis device is flexible, it may be folded onto itself and delivered through a small diameter device in a compacted state where the delivery device covers the anastomosis device and protects body tissue during delivery.

FIG. 75 depicts a further alternative embodiment of an anastomosis device. The device is a flexible cone 4900 having two opposing conical structures 4902, 4904 and flexible tissue engaging structures 4906 to engage adjacent tissue portions. The flexible cone 4900 may be adapted to fit within two adjacent tissue portions to be joined. The tissue engaging structures 4906, extending from the first conical structure 4902 may engage a first tissue portion, while a second tissue portion is engaged by the second conical structure 4904 via a compression engagement, clamping, additional tissue engaging structures (not shown) or other methods disclosed herein or known in the art. In an alternate embodiment of the device depicted in FIG. 75, the conical portions of the device can be constructed from a plurality of discrete struts that attach at one end to the top end of the cone (widest portion of the cone) and at the other end are hingedly attached to each other at a point between the top ends of the cones. The struts would include the tissue securing structures. Because of the hinged attachment, the struts can be bent or collapsed inwardly by the delivery device and the anastomosis device can be delivered to the tissue portions to be joined. Once in place, the anastomosis device can be deployed from the delivery device and the struts extended outward causing the tissue engaging structures to engage the tissue portions, anchoring the anastomosis device in place.

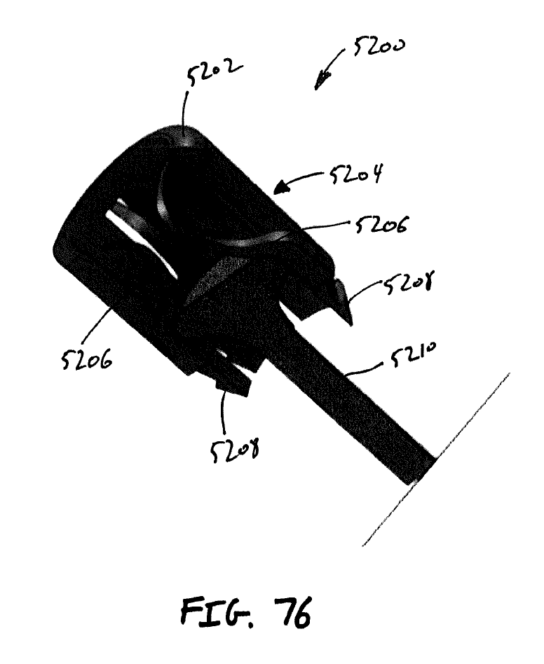

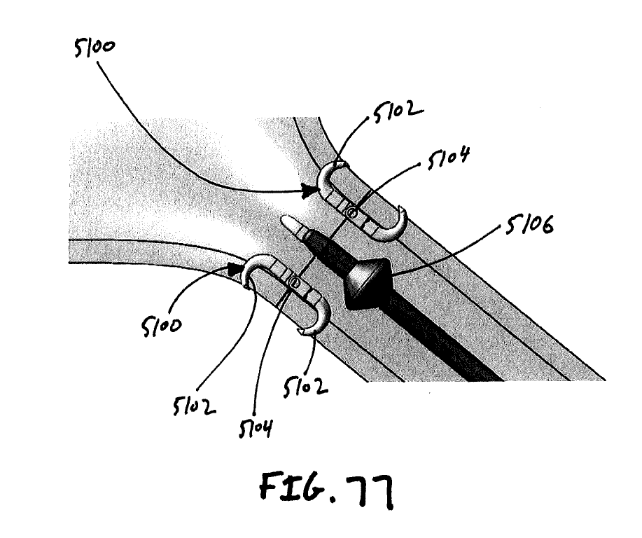

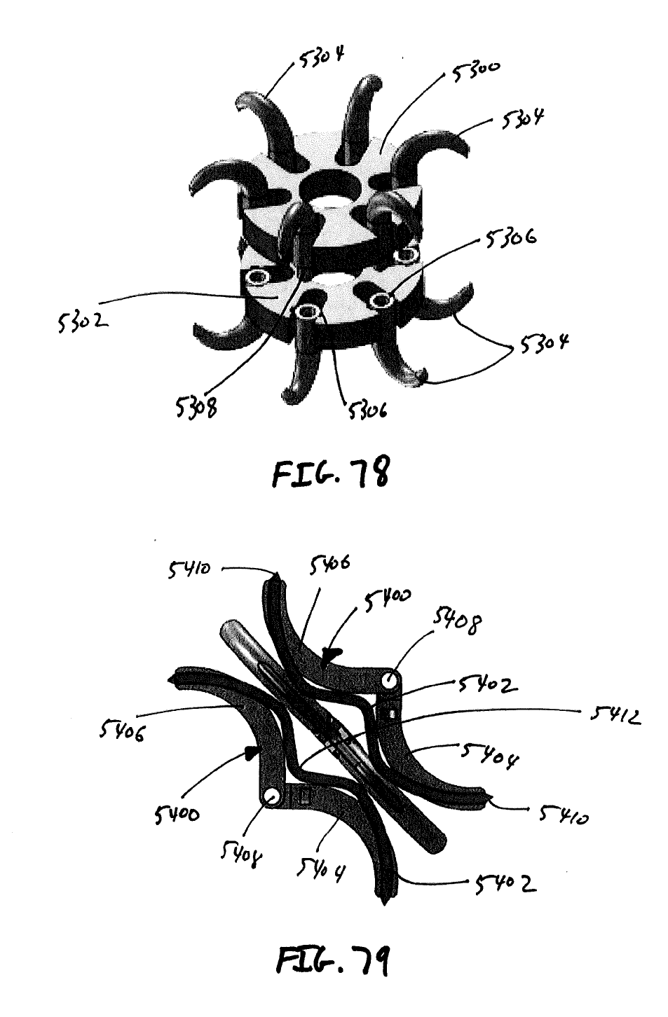

FIG. 76 depicts a further alternative embodiment of an anastomosis device. The device comprises two anastomosis rings 5200 (only one shown) each having a generally cylindrical body 5202 with longitudinal slots 5204 extending there through. Flexible tissue engaging structures 5206 are mounted to the body 5202 via a living hinge or other means to facilitate their deployment through the longitudinal slots 5204. In addition, the rings 5200 include complementary ring connecting structures 5208 for joining the rings together in order to complete the anastomosis (although a male ring connecting structure is shown here, the mating ring may have a corresponding female structure). Once the ring 5200 is inserted to a desired location, the hooks are deployed using a plunger 5210 that engages the inner curve of each hook 5206, forcing them radially outward. There may be an optional locking structure (not shown) that retains the hooks 5206 in a deployed position. The embodiment of FIG. 76 may be used with the insertion devices and procedures disclosed herein, or any other device or procedure that may facilitate delivery of the device 5200, deployment of the tissue engaging structures 5206, and joining of the rings with each other.

FIG. 77 depicts a further alternative embodiment of an anastomosis device. The device comprises a plurality of hinged tissue engaging structures or hooks 5100, each having opposing teeth 5102, joined by a central hinge 5104, which may be a living hinge. The hooks 5100 may be inserted in unison with each other and may be deployed with a deployment mechanism in the form of a plunger 5106 that pivots the teeth 5102 about the hinge 5104 radially outward into adjacent tissue. Optionally, a sheath may be used to facilitate insertion and location of the hooks 5100 prior to deployment.

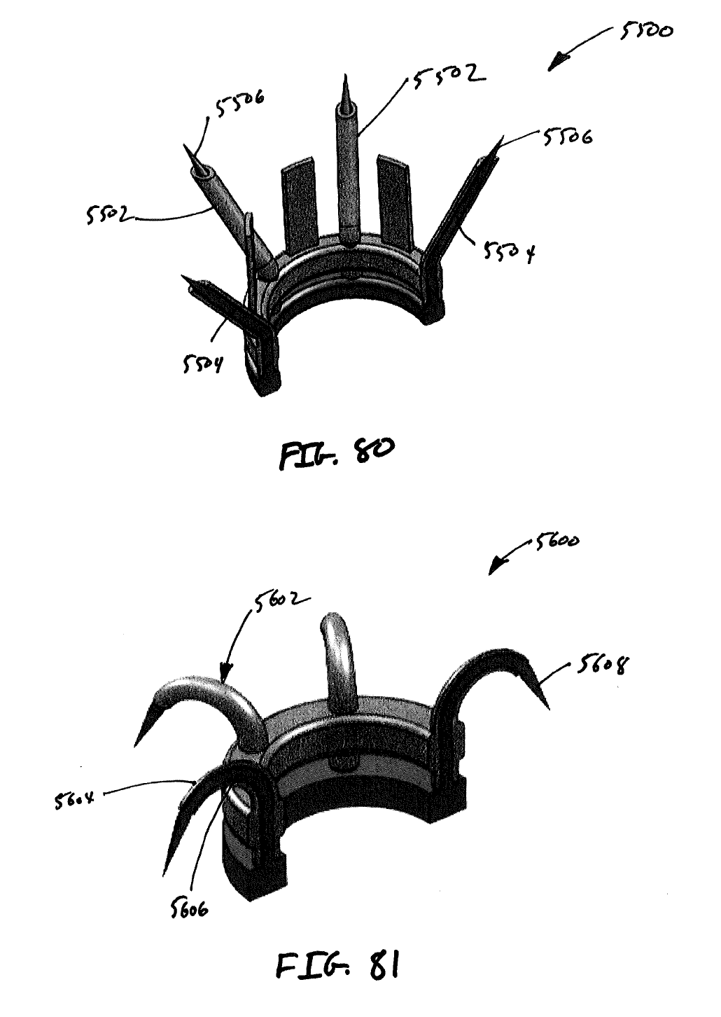

FIG. 78 depicts a further alternative embodiment of an anastomosis device. The device comprises a plurality of mating rings 5300, 5302. Each ring includes a plurality of tissue engaging structures 5304 that are pivotable about a pivot point 5306 from a retracted position (not shown) to a deployed position, as shown in FIG. 78. The tissue engaging structures 5304 may be deployed using a plunger, a cam or other deployer device such as those disclosed herein. In an alternate embodiment of the device depicted in FIG. 78, the tissue engaging structures 5304 of the mating rings 5300, 5302, can include joining structures 5308 that allow the individual tissue engaging structures 5304 of the separate mating rings 5300, 5302 to be joined to each other. In this embodiment, the tissue engaging structures 5304 can be delivered to their respective tissue portions by the mating rings 5300, 5302. Once in place, the tissue engaging structures 5304 can be engaged with their respective tissue portions and then brought into contact with each other with the aid of the mating rings 5300, 5302. When brought into contact with each other, the joining structures 5308 of the tissue engaging structures 5304 will engage each other thereby joining the individual portions of the tissue engaging structures 5304 with each other, completing the anastomosis. The mating rings 5300, 5302 can then be withdrawn leaving only the tissue engaging structures 5304 in place.

FIG. 79 depicts a further alternative embodiment of an anastomosis device. The device comprises a plurality of hinged deployment structures 5400 that are used to deploy a plurality of nitinol tissue engaging structures in the form of staples 5402. The deployment structure 5400 is a two-piece structure having a first arm 5404 and a second arm 5406 that are joined to each other with a hinge 5408 or similar structure. The tissue engaging structures 5402 include two tissue piercing tips 5410. To deploy the tissue engaging structures 5402, the deployment structures 5400 are retracted radially inward thereby forcing the curved portion 5412 of the tissue engaging structure 5402 to invert. This movement causes the tissue engaging structures 5402 to bend radially outward in a curved manner to simultaneously engage the first and second tissue portions similar to how a staple works when stapling paper together. Once the tissue engaging structures 5402 are deployed, the anastomosis is complete. The staples may be inserted using an optional sheath and may be deployed using any of the delivery devices disclosed herein. To add temporary support to the anastomosis, the deployment device 5400 may be left in place.

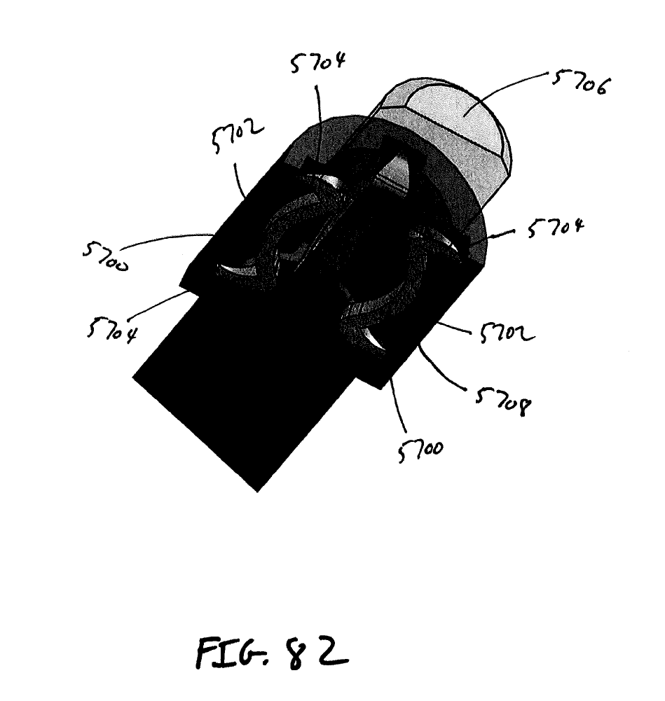

FIG. 80 depicts a further alternative embodiment of an anastomosis device. The device comprises two anastomosis rings 5500 (only one is shown), each including a plurality tissue engaging structures 5502 extending therefrom. The tissue engaging structures 5502 each include a nitinol core 5504 with tissue piercing tips 5506 for piercing adjacent tissue during anastomosis. After insertion into the desired location, the nitinol core 5504 assumes an angled geometry forcing the tissue piercing tips 5506 into adjacent tissue portions thereby anchoring the rings 5500 in place. Once the rings 5500 are anchored in their respective tissue portions, the rings 5500 are brought into contact with each other and joined using a snap-fit or friction-fit connection in order to complete the anastomosis. The embodiment of FIG. 80 may be used with the insertion devices and procedures disclosed herein, or any other device or procedure that may facilitate delivery of the rings 5500.

FIG. 81 depicts a further alternative embodiment of an anastomosis device. The device comprises two anastomosis rings 5600 (only one is shown), each ring 5600 including a plurality tissue engaging structures 5602 extending therefrom, which serve to anchor the rings 5600 to adjacent tissue. The tissue engaging structures 5602 each include a hardened material 5604 with a nitinol core 5606 with tissue piercing tips 5608 for piercing adjacent tissue during anastomosis. The rings 5600 may be operable using a complimentary sleeve or sheath (not shown) to facilitate insertion and deployment. The embodiment of FIG. 81 may be used with the insertion devices and procedures disclosed herein, or any other device or procedure that may facilitate delivery of the rings 5600.

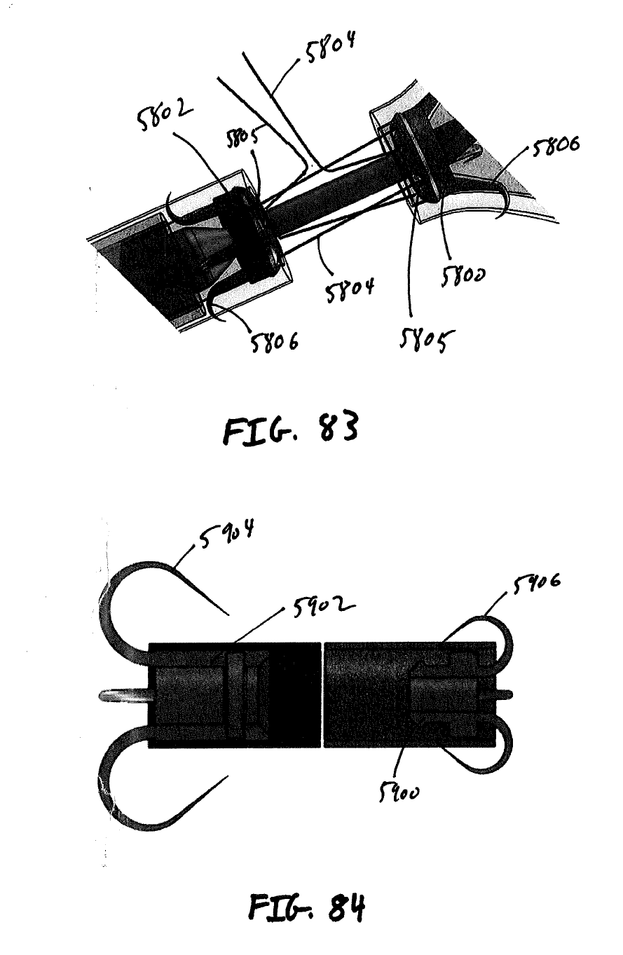

FIG. 82 depicts a further alternative embodiment of an anastomosis device. The device comprises a plurality of individual staple-like tissue engaging structures 5700 each having a curved center section 5702 and opposing teeth 5704 located at each end of the center section 5702. The tissue engaging structures 5700 may be deployed using a plunger-like device 5706 and sheath 5708, such that when the tissue engaging structures 5700 are positioned, the sheath 5708 may be removed and the plunger 5706 may be used to urge the tissue engaging structures 5700 outwardly into adjoining tissue to secure two adjoining tissue portions together. The embodiment of FIG. 82 may be used with the insertion devices and procedures disclosed herein, or any other device or procedure that may facilitate delivery of the tissue engaging structures 5700.

FIG. 83 depicts a further alternative embodiment of an anastomosis device. The device comprises first and second anastomosis rings 5800, 5802 joined by one or more sutures 5804. Each ring 5800, 5802 includes a plurality of suture apertures 5805 to receive the sutures 5804 in order to join or tie the rings 5800, 5802 together. Each ring 5800, 5802 also includes a plurality of tissue engaging structures 5806, adapted to engage adjacent tissue to secure or anchor the respective ring 5800, 5802 thereto. After the anastomosis rings 5800, 5802 are anchored in place in their respective tissue portions, the sutures 5804 are drawn tight in order to pull the anastomosis rings 5800, 5802 toward each other. Once the rings are properly positioned with respect to each other, the sutures can be tied thereby completing the anastomosis. The embodiment of FIG. 83 may be used with the insertion devices and procedures disclosed herein, or any other device or procedure that may facilitate delivery of the rings 5800, 5802 and deployment of the tissue engaging structures 5806.

FIG. 84 depicts a further alternative embodiment of an anastomosis device. The device comprises first and second anastomosis rings 5900, 5902 adapted to matingly engage each other. Each ring 5900, 5902 includes a plurality of tissue engaging structures 5904, 5906 adapted to engage adjacent tissue, securing or anchoring the respective ring 5900, 5902 thereto. The tissue engaging structures 5904, 5906 are preferably curled outward, and may be biased with an inward pressure to pierce adjacent tissue and draw it radially inward towards the rings 5900, 5902. Also, as depicted in FIG. 84, the tissue engaging structures 5904, 5906 may be of different sizes, depending on the type of body tissue to be engaged. The rings 5900, 5902 are secured to each other using a male and female friction or snap-fit connection that is adjustable on order to control the magnitude of force between the contacting tissue portions. The embodiment of FIG. 84 may be used with the insertion devices and procedures disclosed herein, or any other device or procedure that may facilitate delivery of the device, deployment of the tissue engaging structures 5904, 5906, and joining of the first and second rings 5900, 5902 to each other.

The embodiments disclosed herein with respect to 45A to 84 may be used in conjunction with other embodiments of devices and methods disclosed herein.

In all embodiments of the anastomosis rings, collars or devices disclosed herein, holes or apertures may be included in the walls of the device in addition to any apertures provided for tissue engaging structures to extend through in order to allow tissue ingrowth to promote either healing or to further anchor the device.

The preferred materials for the ring assembly 3 are now discussed. However, it will be understood that this discussion of materials can apply equally to all embodiments disclosed and contemplated herein. The ring assembly 3 is preferably formed of materials that are compatible with the environment (e.g. range of pH, variable constituents of bodily fluids such as urine and variable flow of such fluids). The entirety of the ring assembly 3 may be formed from resorbable material(s) or at least a portion of the assembly may be formed from permanent material(s). Alternatively, one or more portions of the ring assembly 3 may be formed of resorbable material(s) while one or more other portions are formed from permanent material(s). In some embodiments, the first ring and second ring securement elements 20 and 62, in particular, are formed from resorbable material, whereas other portions are formed from permanent materials. In some examples, a ring assembly 3 can be formed with a resorbable element that connects two non-resorbable elements and breaks down to permit the ejection of the permanent elements in the urine stream. In other examples, portions of the ring assembly may be formed from mixtures of different resorbable materials and/or different permanent materials.

As used herein, “permanent materials” refers to materials that are not expected to undergo dramatic changes in strength or composition during the period of time that the ring assembly 3 is needed to allow healing of tissues and the establishment of a tissue-based channel for urine flow. Permanent materials include, but are not limited to, polymeric materials or metals. Examples of permanent polymeric materials include PEEK (polyether ether ketone), polyethylene, polypropylene and others currently used in medical devices both in the United States and worldwide. Permanent metals include those used in surgery such as, but not limited to, stainless steel and titanium, both in a range of compositions and alloys.

As used herein, “resorbable materials” refers to materials that exhibit the ability to change over time, such as breaking down and eventually being eliminated from the body of the patient. Resorbable materials include, but are not limited to, bioabsorbable and biodegradable materials. Preferably, resorbable materials may be used as elements of implantable devices where over a period of time the implant breaks up and is absorbed, shed, or ejected from the body.

Resorbable materials are well known in the literature. See, Principles of Tissue Engineering (Lanza and Vacanti, eds., Elsevier Academic Press 3d ed. 2007) (1997), incorporated herein by reference in its entirety. Suitable resorbable materials include, but are not limited to, homopolymers and co-polymer blends from families including polylactic acid, polyglycolic acid, ε-caprolactone, and trimethylene carbonate. Other resorbable polymers may include polyphosphazenes, polydioxanones, polyanhydrides and polyurethane materials. Additionally, materials based on naturally occurring substances including, but not limited to polyhydroxyalkanoates, chitin and its derivatives, cellulose and certain other starches that can be fabricated to useful forms may be used. Additionally, suitable resorbable materials may comprise metals, such as magnesium, that can be broken down by the body when used as an implantable device. In one embodiment of the device, representative resorbable materials may comprise blends of 10:90 and 50:50 (both polyglycolide:polylactide blends), which are materials with degradation times that vary from 1-3 months. Alternatively, representative resorbable materials may comprise blends of 82:18 or 85:15 (both polyglycolide:polylactide blends), which are materials with degradation times that vary from 6-12 months. Material degradation times may be altered by changing processing methods (including exposure to heat and/or moisture during or after processing) as well as sterilization method. Also, environmental characteristics, such as pH and temperature, will also affect implant characteristics, such as degradation time.

Additionally, the ring assembly may be formed from ceramics, such as calcium phosphate and hydroxyapatite based ceramics. By way of background, see e.g., Biomaterials Science: An Introduction to Materials Medicine 64-73 (Buddy D. Ratner ed., Academic Press, Ltd., 1996), incorporated herein by reference in its entirety. The ceramic materials may be permanent or resorbable depending on their chemistry, blending and even manufacturing methods used. The ring assembly 3 may also be formed of a biocompatible, resorbable and/or permanent materials, such as those described in the following US patents, the contents of which are incorporated by reference in their entirety herein: U.S. Pat. No. 5,432,395, U.S. Pat. No. 4,976,715, U.S. Pat. No. 5,273,964, U.S. Pat. No. 4,157,378, U.S. Pat. No. 4,429,691, U.S. Pat. No. 4,612,053, U.S. Pat. No. 4,684,673, U.S. Pat. No. 4,737,411, U.S. Pat. No. 4,849,193, U.S. Pat. No. 4,880,610, U.S. Pat. No. 4,917,702, U.S. Pat. No. 4,938,938, U.S. Pat. No. 4,959,104, U.S. Pat. No. 5,034,059, U.S. Pat. No. 5,037,639, U.S. Pat. No. 5,047,031, U.S. Pat. No. 5,053,212, U.S. Pat. No. 5,085,861, U.S. Pat. No. 5,129,905, U.S. Pat. No. 5,149,368, U.S. Pat. No. 5,152,836, U.S. Pat. No. 5,164,187, U.S. Pat. No. 5,178,845, U.S. Pat. No. 5,262,166, U.S. Pat. No. 5,279,831, U.S. Pat. No. 5,281,265, U.S. Pat. No. 5,286,763, U.S. Pat. No. 5,336,264, U.S. Pat. No. 5,427,754, U.S. Pat. No. 5,470,803, U.S. Pat. No. 5,496,399, U.S. Pat. No. 5,516,532, U.S. Pat. No. 5,522,893, U.S. Pat. No. 5,525,148, U.S. Pat. No. 5,542,973, U.S. Pat. No. 5,545,254, U.S. Pat. No. 5,562,895, U.S. Pat. No. 5,565,502, U.S. Pat. No. 5,605,713, U.S. Pat. No. 5,650,176, U.S. Pat. No. 5,665,120, U.S. Pat. No. 5,691,397, U.S. Pat. No. 5,700,289, U.S. Pat. No. 5,782,971, U.S. Pat. No. 5,846,312, U.S. RE33,161, U.S. RE33,221, U.S. Pat. No. 5,658,593, U.S. Pat. No. 6,752,938, U.S. Pat. No. 8,048,443, and U.S. Pat. No. 8,048,857.

In all of the disclosed ring assembly embodiments disclosed herein, a sealant can be included between the first ring assembly and the second ring assembly for sealing the ring assemblies together. Any such sealant can be moisture activated. Moreover, the sealant may be a 2-part product that only activates when the two parts are in contact similar to a 2-part epoxy. Thus, if a 2-part sealant is used, one part can be included on the first ring assembly and the other part can be included on the second ring assembly such that when the first and second ring assemblies are coupled together, the two parts will contact each other and activate thereby sealing the assemblies together.

As mentioned above, when the ring assembly 3 is formed from resorbable and/or biodegradable materials, it gradually degrades after implantation in the body. Preferably, the material is selected to degrade at a slower rate than the natural healing process, so as to allow healing of the anastomosis before degradation. For example, the ring assembly 3 can be formed from a material that will (i) remain intact for approximately six weeks after implantation before degradation and (ii) be completely resorbed or degraded after twelve weeks. Thus, the ring assembly 3 can be removed or expelled from the patient's body without a follow-up surgical procedure when the ring assembly 3 is no longer needed to hold the anastomosis. In the interim, the ring assembly 3 permits bodily fluids, such as urine, to flow from the first hollow body part, such as a bladder, through the lumens (10, 35 60, and 80) of the first and second ring assemblies 2, 52 and into the second hollow body part (e.g., urethra) while the anastomosis is healing. Preferably, the ring assembly 3 forms a leak-proof passageway, so as to reduce or eliminate the chance of leakage of urine into the abdominal cavity. The flow of bodily fluid, such as urine, through the ring assembly may operate to degrade the ring assembly and carry non-resorbed materials and portions of the ring assembly out of the body.

It will be noted that in some other embodiments, the mating screw threads can be reversed so that the operations described are performed by rotating the components in the opposite angular directions. In some other embodiments, the ring-mounting steps and the securement element-deploying steps can be performed by other components of the system. In some other embodiments, the securement elements can be spring-biased to their deployed positions and deployed by actuation of a release member.

It should be understood that, although this disclosure describes different embodiments separately, that one skilled in the art may combine the features of different embodiments without departing from the anastomosis devices and system disclosed herein. For example, one skilled in the art may incorporate the securement elements and deployment mechanism of one embodiment in a first ring assembly (e.g., rigid pivotable hooks, etc.) while incorporating a different securement element and deployment mechanism (e.g., resilient flexible hooks, etc.) in the second ring assembly. Furthermore, it should be apparent to those skilled in the art that the tissue capture elements referred to as “upper” and “lower” may be adapted for use interchangeably. In other words, a first ring shown engaging the bladder or described as “upper” may be adapted to engage the urethra or used as a “lower” ring. Likewise, a second ring shown engaging the urethra or described as “lower” may be adapted to engage the bladder or used as an “upper” ring.

It should also be understood that although the present disclosure may describe deployment or actuation of certain structures by moving or translating a component or structure distally or proximally with respect to another component or structure, those skilled in the art will understand that deployment of the same structures may be accomplished by moving or translating such components in a different manner. For example, while the present disclosure may describe deploying securement elements by moving a central ring proximally towards an upper collar, deploying the same securement elements may be achieved by moving the upper collar distally towards the central ring. Moreover, it should be understood that although the present disclosure describes deployment of certain structures as occurring when one component is moved towards another component that is held stationary, those skilled in the art will understand that the deployment of such structures, may be accomplished by moving both components towards each other.

Additionally, all US patents, applications, and published literature cited herein are incorporated by reference in their entireties.

It is to be understood that this invention is not limited to the specific devices, methods, conditions, or parameters described and/or shown herein, and that the terminology used herein is for the purpose of describing particular embodiments by way of example only. Thus, the terminology is intended to be broadly construed and is not intended to be limiting of the disclosed invention. For example, as used in the specification including the appended numbered paragraphs, the singular forms “a,” “an,” and “one” include the plural, the term “or” means “and/or,” and reference to a particular numerical value includes at least that particular value, unless the context clearly dictates otherwise. In addition, any methods described herein are not intended to be limited to the sequence of steps described but can be carried out in other sequences, unless expressly stated otherwise herein. And any dimensions shown in the attached drawings are representative and not limiting of the invention, as larger or smaller dimensions can be used as desired.

Although the present invention has been described above in terms of exemplary embodiments, it is not limited thereto. Rather, the appended numbered paragraphs should be construed broadly to include other variants and embodiments of the invention which may be made by those skilled in the art without departing from the scope and range of equivalents of the invention.

1. A two-part anastomosis assembly for connecting a first tissue portion to a second tissue portion, the anastomosis assembly comprising:
a first anastomosis ring having first tissue engaging structures for deployment, by actuation of a deployment mechanism of a deployment device, to attach to the first tissue portion; and
a second anastomosis ring having second tissue engaging structures for deployment, by actuation of a deployment mechanism of a deployment device, to attach to the second tissue portion;
wherein the first and second anastomosis rings include interconnecting elements for joining the first and second anastomosis rings together, and
wherein, during delivery of the first and second anastomosis rings, the first and second tissue engaging structures are contained within an inner diameter of the first and second anastomosis rings.

2. An anastomosis assembly for connecting a first tissue portion to a second tissue portion, the anastomosis assembly comprising:
a first anastomosis portion having first tissue engaging structures for deployment, by actuation of a deployment mechanism of a deployment device, to attach to the first tissue portion; and
a second anastomosis portion having second tissue engaging structures for deployment, by actuation of a deployment mechanism of a deployment device, to attach to the second tissue portion,
wherein, during delivery of the anastomosis assembly, the first and second tissue engaging structures are contained within an inner diameter of the first and second anastomosis portions.
